(19) United States
(12) Patent Application Publication
(10) Pub. No.: US 2007/0059272 A1
(43) Pub. Date: Mar. 15, 2007

(54) Modulation of microbial pathogen-host cell interactions

(75) Inventor: John C. Alverdy, Glenview, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: The University of Chicago, Chicago, IL (US)

(21) Appl. No.: 11/445,743

(22) Filed: Jun. 2, 2006

Related U.S. Application Data
(60) Provisional application No. 60/687,537, filed on Jun. 3, 2005.

(51) Int. Cl. G01N 33/554; A61K 31/787; A61K 31/485

(52) U.S. Cl. 424/78.3; 435/7.32; 514/282

(57) ABSTRACT

The invention provides methods of screening for modulators of microbial PA-I lectin/adhesin activity, including modulators of PA-I expression, as well as the modulators so identified, pharmaceutical compositions and kits containing such modulators. These modulators include soluble and membrane-bound bacterial signaling compounds produced by cells of a host containing a microbial pathogen. Methods for preventing and treating cell disorders, such as epithelial cell disorders including gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly or pig-bel, Pseudomonas-mediated ophthalmologic infection, Pseudomonas-mediated otologic infection and Pseudomonas-mediated cutaneous infection, using the modulators are contemplated, as are methods for ameliorating a symptom associated with such a disorder.

### CROSS REFERENCE TO RELATED APPLICATION

This application claims the benefit of Provisional U.S. Patent Application No. 60/687,537, filed Jun. 3, 2005, the entirety of which is expressly incorporated herein by reference.

The invention was made with U.S. Government support under contract no. GM62344 awarded by the National Institutes of Health. The U.S. Government has certain rights to this invention.

### FIELD OF THE INVENTION

The invention generally relates to the field of prophylactic and therapeutic modulation of microbial pathogen involvement in disorders and diseases of mammals such as humans.

### BACKGROUND

Pseudomonas aeruginosa is a ubiquitous bacterium found in soil, water, and plants and can be part of the normal flora in humans. In the context of human infection, it is an opportunist which causes disease primarily in patients with other underlying disease states. During physiologic stress, as occurs during critical illness or severe inflammation, the presence of P. aeruginosa in the lung or intestine of patients is associated with a poor prognosis and can cause lethal pneumonia or gut-derived sepsis. P. aeruginosa is the most common gram-negative bacterium isolated among cases of nosocomial infection and carries the highest reported fatality rate of all hospital acquired infections¹. The mere presence of this pathogen within the intestinal tract of a critically ill patient is associated with a four fold increase in mortality, independent of its dissemination to remote organs². It is generally reasoned that the observed increase in mortality of critically ill patients colonized by powerful nosocomial pathogens is simply due to lowered host resistance. Yet an alternative explanation for the continued high mortality rate among such severely stressed patients, despite more powerful antibiotics, is that certain opportunistic pathogens have evolved highly sophisticated mechanisms to sense both the susceptibility and liability of a physiologically stressed host and respond with enhanced virulence.

Despite improvements in antibiotics and patient care, mortality rates from severe sepsis following surgical injury and infection have remained unchanged for decades. Among patients at risk for severe sepsis, it has become axiomatic that those subjected to the greatest degree and number of physiologically stressful insults (termed “hits”), suffer the greatest mortality³. While advances in medical care can rescue patients from many of these physiologic “hits”, the incidence and mortality from sepsis has been held constant by the survival of patients subjected to unprecedented levels of catabolic stress who become subsequently colonized by some of the most virulent and resistant nosocomial pathogens known to man⁴. The intestinal tract reservoir is the anatomic site on which physiologic stress exerts a profound effect on organ function, and in which the greatest microbial burden accumulates through the course of such stress⁵. That intestinal pathogens acquired during the course of critical illness might play a major role in the mortality due to sepsis has led to the term gut-derived sepsis⁶. Since as many as 30% of critically ill patients with severe sepsis have no identifiable pathogen or focus of infection, the intestinal microflora could play a major role in driving and sustaining the systemic inflammatory response^{7,8}.

A recent surveillance study of 35,790 non-duplicate, gram-negative aerobic bacteria isolated from intensive care units (ICUs) in 43 US states identified P. aeruginosa as the most common gram-negative species isolated among critically ill patients⁹. The mere presence of P. aeruginosa in the intestinal tract of a critically ill host is associated with a 4-fold increase in mortality². Antibiotic resistance to this pathogen has increased 70% over the last 5 years¹⁰ and crude mortality statistics demonstrate that P. aeruginosa continues to be associated with the highest case fatality rate of all nosocomially acquired infections¹¹. Numerous studies have now confirmed that the intestinal tract is the single most important reservoir for subsequent infection with this pathogen¹². A direct role for intestinal P. aeruginosa in the mortality of critically ill patients is supported by a recent prospective randomized trial of approximately 800 critically ill patients, half of whom where treated with non-absorbable oral antibiotics to decontaminate the intestinal tract of its flora. A statistically significant reduction in mortality (about 17%) was observed in antibiotic decontaminated patients in association with a significant reduction in antibiotic resistant strains of P. aeruginosa isolated from feces¹³. Yet precisely how intestinal P. aeruginosa contributed to the mortality of these critically ill patients remains unknown.

Microbial pathogens such as P. aeruginosa can express a protein termed PA-I lectin/adhesin (PA-I). PA-I is a lectin comprised of four 13 kDa subunits, that bind to D-galactose and its derivatives¹⁹. Among monosaccharides, the binding specificity is strictly for galactose, with the exception of N-acetyl-D-galactosamine (GalNAc)²⁰. Disaccharides containing a terminal α-D-galactose residue bind PA-I. The highest binding affinity to date has been demonstrated for the disaccharide mellibiose (α-GA11-6GIC)²¹. P. aeruginosa strains suspended in a 0.6% solution of the specific PA-I binding sugar GalNAc or the disaccharide mellibiose, have markedly decreased adhesion to intestinal epithelial cells and do not alter their barrier function²⁰. PA-I's distribution in bacteria can be either primarily intracytoplasmic or extracellular, depending on its environment. When bacteria are grown in ideal growth conditions, about 85% of PA-I is located intracellularly with small, but significant, amounts located within the cytoplasmic membrane, on the outer membrane, and in the periplasmic space²². In sharp contrast, within the intestinal tract of a stressed host, PA-I abundance is increased and localizes to the outer membrane facilitating the adherence of P. aeruginosa to the intestinal epithelium¹⁵. In addition, there is evidence that free PA-I is shed into the extracellular milieu and can be detected at concentrations as high as 25 μg/ml in both culture supernatants and sputum from P. aeruginosa infected lungs. This finding is of considerable importance, as treatment of cultured epithelial cells (e.g. T-84, Caco-2bbe, MDCK, airway epithelial cells) with 25 μg/ml purified PA-I, causes a profound permeability defect²⁴. This effect is also seen in the intestinal tract in vivo¹⁴.

In P. aeruginosa, PA-I expression is under the regulatory control of two important systems of virulence gene regulation: the quorum sensing signaling system and the alternative sigma factor system, termed RpoS²⁶. P. aeruginosa has an exceptional ability to sense and respond to changes in its local microenvironment and switch on and off its virulence genes accordingly. Because of their importance, these two mechanisms of gene regulation are described in the following sections.

Quorum sensing signaling system. The plasticity of P. aeruginosa gene expression is achieved, in part, via the well described quorum sensing signaling system. The term quorum sensing arose from the observation that certain pathogens activate their virulence genes only when they reach a critical population density (i.e., quorum), presumably that amount necessary to mount an overwhelming attack on the host. Small molecules termed pheromones or “autoinducers” (homoserine lactones-HSLs), are self-generated by the bacterial cells and accumulate in the environment when bacteria achieve a high population density, as occurs in late stationary phase of growth. Once an intracellular threshold level of an autoinducer is reached, the autoinducer (C4-HSL, C12-HSL) binds to its cognate transcriptional regulator protein (LasR, RhIR) to activate or repress target genes. This process was first described in Vibrio Fisheri as regulating the lux genes responsible for bioluminscence²⁷. P. aeruginosa possesses one of the most highly characterized models of QS, and two complete lux-like QS systems, lasR-lasI and rhlR-rhlI have been identified. Quorum sensing (QS) in P. aeruginosa controls the cell-to-cell communication pathways that direct its complex assemblage behavior including motility, biofilm synthesis, clumping, PA-I expression, and the secretion of a variety of cytotoxic exoproducts (exotoxin A, proteases, rhamnolipids, pyocyanin, elastase)²⁸. Data from many models of both acute infection and chronic infection have supported the hypothesis that QS is important in P. aeruginosa pathogenesis²⁸.

RpoS signaling system. A second major regulatory system used by P. aeruginosa is the RpoS (σ^{S}) system. Bacterial σ (sigma) factors are positive regulators of gene expression that direct the initiation of transcription through direct binding to promoter sequences and recruitment of core RNA polymerase²⁶. The σ factor RpoS (σ^{S}), was originally identified in E. coli as an alternative a factor that activates gene expression in stationary phase when cells are experiencing nutrient starvation. Now σ^{S} is considered to be a master stress response regulator important for adaptation to a variety of conditions, including hyperthermia and oxidative stress²⁹. There is considerable evidence linking the RpoS response to QS in P. aeruginosa³⁰⁻³⁴. The rhl quorum-sensing system has been reported to activate the transcription of rpos, and recently Greenberg reported that RpoS affects the expression of more than 40% of all quorum-controlled genes²⁶. A master regulator like RpoS (σ^{S}) can commit a bacterial cell to a certain complex developmental program with specific temporal and spatial control being exerted by various secondary regulatory systems (FIGS. 1 and 2).

Microarray technology has enabled studies aimed at improved understanding of the molecular mechanisms of virulence gene expression in P. aeruginosa³⁵. Results from three independent studies demonstrate that both RpoS and QS play a key and interconnected role in the activation of virulence gene expression in P. aeruginosa²⁶. In addition, a key virulence gene in P. aeruginosa gut-derived sepsis, lec A (encoding PA-I) was one of only two genes shown to be directly regulated by both systems and ranked among the top 1% of QS-dependent virulence genes based on inducible transcript accumulation^{26,30}. The following is a brief description of these studies.

Greenberg and his colleagues recently performed a comprehensive analysis to identify genes regulated by RpoS in P. aeruginosa²⁶. Specifically, they compared the transcript profiles of an rpoS mutant with its parent at specific cell densities. 772 genes and 13 intergenic regions were identified that showed differential expression in stationary phase. Of the 772 genes examined, RpoS induced 504 and repressed 268. Remarkably, the PA-I gene, termed lecA, showed a 20-fold increase in expression over the rpos mutant in stationary phase of growth. In a separate microarray analysis of P. aeruginosa QS regulons, this same laboratory examined transcript abundance to exogenous HSL's (C4-HSL+C12-HSL) in wild type versus a lasRI and rhIR mutant strain. Mutants lacking lasRI and rhIR will respond poorly to their cognate QS signaling molecules, whereas wild type strains will increase transcript levels in response to exogenous HSL's. The ratio of wild type to mutant MRNA will therefore reflect increases in gene expression which are QS dependent. Among the 638 genes that showed a maximal response to exogenous HSL's of at least 2.5 fold, PA-I expression increased to a maximum of 200 fold, ranking in the top 10 inducible genes based on transcript accumulation. This study also demonstrated that lecA (which codes for the PA-I) was one of only two genes with upstream regulatory regions containing both las-rhl box sequences and RpoS sequences.

Iglewski similarly examined the effects of growth phase and environment on the P. aeruginosa QS regulon using microarray analysis in strains exposed to exogenous HSL's³⁶. Media composition and oxygen availability were also examined for their effects on virulence gene expression. In this study, 616 genes were identified as being QS regulated. Only 5 genes exhibited increases of 60 fold or more in response to exogenous HSL; the PA-I was included in this high expressing group. Interestingly, the lec A gene (encoding for PA-I) was not expressed in a hypoxic/anaerobic environment.

The interconnectedness of QS and Rpos in P. aeruginosa provides a level of redundancy, additiveness, and internal feedback regulation that has armed this pathogen with signal-integrative power like no other microbe, thus accounting for its remarkable success as a lethal pathogen.

Diggle and Winzer have confirmed the above findings and identified yet a third system, the Pseudomonas Quinolone Signal (PQS) that can also activate PA-I expression by interacting directly with both the RpoS and QS systems³⁴.

Microbiologists have long recognized that many bacteria activate their virulence genes in response to ambient environmental cues³⁷. In general such physico-chemical cues signal environmental stress or adversity, such as changes in redox status, pH, osmolality, etc.

Although there has been very little work on specific membrane sensors that activate virulence gene expression in P. aeruginosa, two sensor proteins located within the cell membrane of P. aeruginosa, termed CyaB⁴¹, GacS^{39,42} have been shown to respond to three known external signals, host cell contact, low calcium⁴³, and beet seed extract³⁹. CyaB (via cAMP) and GacS⁴⁴ (via phosphorylation), activate the transcriptional regulators Vfr⁴⁵ and GacA^{42,46} respectively, which along with the cell density sensitive PcrA, exert global regulatory influences on two central systems for virulence gene regulation in P. aeruginosa, the QS and Rpos signaling systems. Mutant strains defective in CyaB and GacS have attenuated lethality in mice following lung instillation⁴⁷.

Host cellular elements such as seed extract and cell contact, activate the membrane biosensors CyaB and GacS. These two component transmembrane alarm systems then activate two main global regulators of virulence, Vfr and GacA. Vfr is involved in the activation of LasRI which in turn promotes the activation of the RhiRI system of QS. GacA induces the transcription of lasR and rhiR genes, and is also implicated in the expression of rpoS. Finally a third system PQS, induces expression of both RhIR and RpoS. Thus activation of any of the membrane biosensors could lead to the expression of PA-I with the involvement of a number of different pathways.

Opioids are distributed in virtually every tissue of the body and are abundantly released in response to various stress conditions (S. Yoshida, et al., Surg Endosc 14, 137 (2000), C. Sternini, S. Patierno, I. S. Selmer and A. Kirchgessner, Neurogastroenterol Motil 16 Suppl 2, 3 (2004)). Morphine and morphine-like compounds are among the most widely used analgesic drugs in the world and are often administered at high doses even at continuous dosing intervals in the most highly stressed critically ill patients. Intravenously applied morphine has been demonstrated to accumulate at tissues sites of bacterial infection such as the intestinal mucosa, at concentrations as high as 100 μM (P. Dechelotte, A. Sabouraud, P. Sandouk, I. Hackbarth and M. Schwenk, Drug Metab Dispos 21, 13 (1993)) and has been shown to readily cross the intestinal wall into the lumen (M. M. Doherty and K. S. Pang, Pharm Res 17, 291 (2000)). Therefore it is likely that an opportunistic pathogen such as P. aeruginosa, which is present in greater than 50% of the intestines of critically ill patients within 3 days of hospitalization, is exposed to both endogenously released and exogenously applied opioid compounds. Within the intestinal tract reservoir, dynorphin and β-endorphin appear to be the predominantly released opioids following stress (C. Sternini, S. Patierno, I. S. Selmer and A. Kirchgessner, Neurogastroenterol Motil 16 Suppl 2, 3 (2004)).

The association of opioids and infection is well established (J. M. Risdahl, K. V. Khanna, P. K. Peterson and T. W. Molitor, J Neuroimmunol 83, 4 (1998)), but most of the work in this area has focused on the suppressive effects of opioids on the immune system (L. K. Eisenstein, et al., Adv Exp Med Biol 493, 169 (2001)). Although opioids have been shown to suppress a variety of immune cells resulting in impaired clearance of bacteria and enhanced mortality in animals (J. Wang, et al., J Leukoc Biol 71, 782 (2002)), it has not been previously considered that opioid compounds might also directly activate the virulence of bacteria.

While in vitro assays have been enormously useful and continue to provide important information on the mechanisms of bacterial pathogenesis, they cannot accurately reproduce all aspects of the host pathogen interaction, as a pathogen will encounter several radically different environments in the host at various points during infection. Consequently, a gene that seems important in in vitro studies, may not be important in vivo, and genes that appear unimportant in an in vitro assay may play a critical role during a natural infection. Furthermore, it has recently been shown that bacteria growing on the surface of solid agar have a markedly different physiology from those in broth, as judged by differential regulation of nearly one-third of their functional genome. Therefore, experiments must now be designed that control for the variables of the growth environment and host environment, while at the same time allowing for measurements of gene expression patterns and phenotype analysis which are not possible in more traditional models, such as stressed mice.

Severe sepsis continues to be the number one cause of mortality among critically ill patients. Interventions to attenuate regulatory arms of the systemic immune response have resulted in clinical failure. Alternatively, newer and more powerful antibiotics have resulted in the emergence of highly resistant stains of bacteria for which there is no foreseeable therapy other than de-escalating their use. P. aeruginosa is now on the international “hit list” of emerging resistant pathogens posing a real and present danger to the public⁵⁷⁻⁶².

### SUMMARY

Using a combination of in vivo and molecular methods, surgical stress has been shown to cause the release of host cell-derived Bacterial Signaling Compounds (host stress-derived BSCs) into the intestinal lumen that directly activate the virulence machinery of P. aeruginosa. The release of such host-derived BSCs, which include morphine, κ and δ opioid receptor agonists, and Interferon gamma (IFN-γ), can shift the phenotype of P. aeruginosa from that of indolent colonizer to lethal pathogen. Exposure of P. aeruginosa to host stress-derived BSCs induces the expression of the PA-I lectin/adhesin (PA-I), a key virulence gene involved in lethal gut-derived sepsis in mice. In at least some instances, induction of PA-I expression is mediated by a transcriptional regulator of virulence gene expression, MvfR. PA-I induces an epithelial permeability defect to at least two potent cytotoxins of this organism, exotoxin A and elastase, causing lethal gut-derived sepsis and other disorders characterized by an epithelial cell barrier dysfunction. The data provide evidence for a model in which opportunistic pathogens sense host stress and vulnerability by perceiving key mediators released by the host into the intestinal tract during stress, such as the stress resulting from surgery. These host stress-derived compounds directly activate critical genes in P. aeruginosa leading to enhanced virulence.

Opioids, released in increased amount during physiological stress, directly induce the expression of several quorum sensing-dependent virulence factors in P. aeruginosa, such as pyocyanin, biofilm, and the lectin/adhesin PA-I. Specifically, U-50,488 (bremazocine, i.e., trans-3,4-dichloro-N-methyl-N[2-(1-pyrolidinyl)cyclohexyl]benzeneacetamide methanesulfonate), an exemplary κ-opioid receptor agonist, induces pyocyanin production in P. aeruginosa via the global virulence transcriptional regulator MvfR. U-50,488 also induces pyocyanin at cell densities below those that would normally produce pyocyanin. These findings indicate that opioids function as host stress-derived bacterial signaling molecules capable of activating a virulence response in P. aeruginosa.

One aspect according to the invention provides a method of screening for a modulator of an epithelial cell barrier function comprising (a) contacting a PA-I lectin/adhesin and an epithelial cell in the presence and absence of a candidate modulator; (b) measuring epithelial cell barrier function; and (c) identifying the candidate modulator as a modulator of an epithelial cell barrier function if the barrier function in the presence of the candidate modulator differs from the barrier function in the absence of the candidate modulator. In preferred embodiments, the modulator reduces the effect of PA-I on epithelial cell barrier function.

In a related aspect, the invention provides a method of screening for a modulator of an epithelial cell barrier function comprising (a) contacting a candidate modulator and an epithelial cell releasing a bacterial signaling compound selected from the group consisting of an opioid, an opioid receptor agonist and interferon-γ; (b) measuring the release of the bacterial signaling compound in the presence or absence of the candidate modulator; and (c) identifying the candidate modulator as a modulator of an epithelial cell barrier function if the level of signal transmission in the presence of the candidate modulator differs from the level of signal transmission in the absence of the candidate modulator. Preferred modulators identified according to the methods of the invention inhibit signal transmission by interacting with the bacterial signaling compound external to the microbial pathogen, e.g., by binding to the bacterial signaling compound. In other embodiments, the modulator binds to the cell surface of a microbial pathogen.

Another aspect of the invention provides a method of screening for a modulator of an epithelial cell barrier function comprising (a) contacting a candidate modulator and a microbial pathogen comprising a functional mvJR coding region under conditions wherein the mvfR is expressed; (b) measuring MvfR expression in the presence or absence of the candidate modulator; and (c) identifying the candidate modulator as a modulator of an epithelial cell barrier function if the level of MvfR in the presence of the candidate modulator differs from the level of MvfR in the absence of the candidate modulator. A preferred microbial pathogen for use in the method is a bacterium, such as gram-negative aerobic rods like a member of the Pseudomonadaceae, e.g., Pseudomonas aeruginosa. In some embodiments, MvfR expression is measured by determining the level of MvfR activity. In other embodiments, MvfR expression is measured by determining the level of expression of a coding region regulated by MvfR. Exemplary coding regions include any of a number of virulence genes endogenous to the microbial pathogen, such as genes found in the PQS, RpoS, or QS regulatory systems. In still other embodiments, MvfR expression is measured by determining the level of a coding region that is not native to the microbial pathogen, such as a chimeric coding region or a heterologous coding region. In these embodiments, expression of the coding region is subject to control, at least in part, by MvfR, preferably by operatively linking an expression control element sensitive to MvfR to the coding region. In preferred embodiments, the modulator inhibits expression of MvfR. The invention comprehends embodiments wherein the conditions comprise exposure to a compound selected from the group consisting of an opioid, an opioid receptor agonist and an interferon-γ, including fragments of such compounds that retain the capacity to influence the level of MvfR activity in a microbial pathogen, e.g., by retaining the capacity to bind or interact with a bacterial receptor or membrane-bound protein (opioid receptor, OprF, and the like).

In a related aspect, the invention provides a method of modulating the activity of MvfR comprising administering a therapeutically effective amount of the modulator identified according to the screening method described above. In some embodiments of this aspect, the method involves direct introduction of the modulator into the intestine of an organism, wherein the intestine is populated by a Pseudomonas aeruginosa cell. A preferred organism is a human being, although mammals such as domesticated animals such as pets, animals of commercial value (e.g., farm animals) and exotic animals (zoo animals) are also contemplated. These methods may further comprise administering a high molecular weight polyethylene glycol-like compound, which is preferably a polyethylene glycol having an average molecular weight of at least 15 kilodaltons.

Another aspect of the invention is drawn to a method of screening for a modulator of an epithelial cell barrier function comprising (a) contacting a candidate modulator and a microbial pathogen comprising a functional PA-I lectin/adhesin coding region under conditions wherein the PA-I lectin/adhesin is expressed; (b) measuring PA-I lectin/adhesin expression in the presence or absence of the candidate modulator; and (c) identifying the candidate modulator as a modulator of an epithelial cell barrier function if the level of PA-I lectin/adhesin in the presence of the candidate modulator differs from the level of PA-I lectin/adhesin in the absence of the candidate modulator. In a preferred embodiment, the microbial pathogen is Pseudomonas aeruginosa. Preferably, the modulator identified by the method inhibits PA-I lectin/adhesin expression or activity. In some embodiments, the conditions comprise induction of PA-I lectin/adhesin expression by a compound selected from the group consisting of an opioid, an opioid receptor agonist and interferon-γ. In some embodiments, the methods will identify a modulator that interferes with interaction between OprF and interferon-γ. In other embodiments, the methods will identify modulators that interfere with the interaction between an opioid receptor and a compound selected from the group consisting of an opioid and an opioid receptor agonist, such as a κ-opioid agonist selected from the group consisting of U-50488, U-69593, enadoline, ethylketocyclazocine, salvinorin A and asimadoline.

A related aspect according the invention provides a method of modulating the expression of PA-I lectin/adhesin comprising administering a therapeutically effective amount of the modulator identified by the above-described screening method. In some embodiments, the step of administering comprises direct introduction of the modulator into the intestine of an organism, preferably a human being, wherein the intestine is populated by a Pseudomonas aeruginosa cell. In some embodiments, the method further comprises administering a high molecular weight polyethylene glycol-like compound, such as a polyethylene glycol having an average molecular weight of at least 15 kilodaltons.

In any of the above-described screening methods, the invention comprehends assessment of an epithelial cell barrier function by direct or indirect microscopic examination of a cellular junction formed between at least two epithelial cells. The invention also comprehends assessment of an epithelial cell barrier function by transepithelial cell electrical resistance (TEER) in any of the aforementioned screening methods. In preferred embodiments where assessment is by TEER, the TEER is higher in the presence of the candidate modulator than in the absence of the candidate modulator.

In another aspect, the invention provides a method of treating a disorder characterized by an epithelial cell barrier dysfunction (partial or complete barrier dysfimction) comprising administering to an organism in need thereof a therapeutically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-1 antagonist, an endomorphine-2 antagonist, an antagonist to δ opioid agonist BW373U86 and the modulator identified by one of the above-described screening methods. In preferred embodiments, the organism in need is a human patient. Contemplated as suitable for treatment is a disorder selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection. In some embodiments, the treatment method further comprises administration of a biocompatible polymer, i.e., a polymer that is compatible with the organism in need in that it is not toxic at the administered level to such an organism and does not elicit a deleterious response in the organism (e.g., an immune response). A preferred biocompatible polymer is a high molecular weight polyethylene glycol-like compound, such as a polyethylene glycol having an average molecular weight of at least 15 kilodaltons.

In some embodiments of the treatment methods of the invention, an opioid receptor antagonist is administered that is an antagonist of a κ-opioid receptor agonist selected from the group consisting of U-50488, U-69593, enadoline, ethylketocyclazocine, salvinorin A and asimadoline. In other embodiments, an opioid receptor antagonist is selected from the group consisting of nor-binaltorphimine, 5′-guanidinonaltrindole, nalmefine, naltrindole, an indolmorphinan, naltrexone and MR2266 ([(−)-(1R, 5R, 9R)-5, 9-diethyl-2-(3-furylmethyl)-2′-hydroxy-6,7-benzomorphan).

In yet other embodiments, a modulator suitable for use in the treatment methods include interferon-γ antagonists, opioid receptor (including κ, δ, and μ opioid receptor) antagonists, an endomorphine-1 (9E1; Tyr-Pro-Trp-Phe-NH₂) antagonist, and an endomorphine-2 (E2; Tyr-Tyr-Pro-Phe-Phe-NH₂) antagonist. An exemplary δ-opioid receptor antagonist is an antagonist to the 6-opioid receptor agonist, BW373U86. Preferred modulators are interferon-γ antagonists and κ-opioid antagonists. An exemplary interferon-γ antagonist is an antibody specifically recognizing interferon-γ, including polyclonal, monoclonal, chimeric, humanized, single-chain and all other forms of antibody known in the art. Contemplated examples of κ-opoid antagonists are nor-binaltorphimine (nor-binaltorphamine, Magers et al., J. Pharmacol. Exp. Ther. 305:323-330 (2003)), 5′-guanidinonaltrindole (Magers et al.), 5′-acetamidinoethylnatrindole (Magers et al.), nalmefine (Culpepper-Morgan et al., Life Sci. 56:187-192 (1995)), naltrindole and indolmorphinans, i.e., compounds comprising a derivatized indole moiety of naltrindole (Stevens et al., J. Med. Chem. 43:2759-2769 (2000)), naltrexone (Craft et al., J. Pharmacol. Exp. Ther. 260:327-333 (1992)), and MR2266 ([(−)-(1R,5R,9R)-5,9-diethyl-2-(3-furylmethyl)-2′-hydroxy-6,7-benzomorphan (Fanselow et al., Beh. Neurosci. 103:663-672 (1989). Additionally contemplated as useful κ-opioid antagonists are specific anti-κ-opioid agonist-recognizing antibodies, including polyclonal, monoclonal, chimeric, humanized, single-chain and all other forms of antibody known in the art. For example, an antibody specifically recognizing the U-50,488 κ-opioid agonist is contemplated by the invention, as are antibodies of any form that specifically recognize any known K-opioid agonist, including but not limited to U-69,593 (Filizola et al., J. Comput. Aided Molec. Des. 15:297-307 (2001)), U-62,066 (spiradoline, Pitts et al., J. Pharmacol.Exp. Ther. 271:1501-1508 (1994)), enadoline (C1-977, Pitts et al.), ethylketocyclazocine (Mello et al., J. Pharmacol. Exp. Ther. 286:812-824 (1998)), salvinorin A, and asimadoline (EMD 61753, Machalska et al., J. Pharmacol. Exp. Ther. 290:354-361 (1999)). Other K-opioid antagonists according to the invention are antibodies of any form that specifically recognize a P. aeruginosa κ-opioid receptor. Each of the references cited in disclosing antagonist modulators is expressly incorporated herein by reference for disclosures relating to such antagonists.

Further contemplated in the treatment methods of the invention are modulators of MvfR-mediated virulence gene expression. Exemplary embodiments of these modulators include compounds that bind to at least one of the substrate binding domain or the DNA binding domain of MvfR. Related embodiments include treatment methods comprising modulators of MvfR-mediated virulence gene expression that inhibit expression of MvfR. The invention also comprehends treatment methods comprising the administration of a therapeutically effective amount of a modulator of PA-I lectin/adhesin activity. Exemplary modulators of PA-I activity are inhibitors of PA-I activity, such as specific anti-PA-I antibodies of any form. Further, such modulators include compounds that reduce the level of expression of PA-I.

Still other treatment methods according to the invention involve administration of a therapeutically effective amount of an anti-sense oligonucleotide capable of specifically hybridizing to a nucleic acid comprising a sequence required for the expression of an opioid receptor, OprF, or MvfR. Related treatment methods involve administration of a therapeutically effective amount of a compound capable of inducing post-transcriptional gene silencing, such as administration of a vector for intracellular expression of a double-stranded RNA homologous to, e.g., the PA-I coding region or an MvfR coding region, or administration of the dsRNA itself, in an RNAi approach to treatment.

In yet another aspect, the invention provides a method of reducing the risk of developing a disorder characterized by an epithelial cell barrier dysfunction comprising administering to an organism at risk of developing the disorder a prophylactically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-l antagonist, an endomorphine-2 antagonist, an antagonist to δ opioid agonist BW373U86 and the modulator identified by the method described above. In some embodiments, an opioid receptor antagonist is selected from the group consisting of a U-50488 antagonist, a U-69593 antagonist, an enadoline antagonist, an ethylketocyclazocine antagonist, a salvinorin A antagonist, an asimadoline antagonist, nor-binaltorphimine, 5′-guanidinonaltrindole, nalmefine, naltrindole, an indolmorphinan, naltrexone and MR2266 ([(−)-(1R,5R,9R)-5,9-diethyl-2-(3-furylmethyl)-2′-hydroxy-6,7-benzomorphan. In preferred embodiments, the organism at risk is a human being, such as a human patient. The methods are contemplated as suitable for reducing the risk of acquiring or developing a disorders selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection. Related aspects of the invention provide methods for reducing the risk of such disorders comprising administrationo f a therapeutically effective amount of a high molecular weight polyethylene glycol-like compound, such as a polyethylene glycol having an average molecular weight of at least 15 kilodaltons, alone or in combination with the administration of a therapeutically effective amount of a compound described above.

Another aspect of the invention is drawn to a method of reducing a symptom associated with an epithelial cell barrier disorder, comprising administering to a patient in need thereof a compound selected from the group consisting of an opioid receptor antagonist, an interferon-γ antagonist, an MvfR antagonist, a regulator of MvfR expression, a PA-I antagonist, a regulator of PA-I expression, an endomorphine-l antagonist, an endomorphine-2 antagonist, an antagonist to δ opioid agonist BW373U86 and a modulator identified according to one of the above-described screening methods, wherein the compound is administered in an amount effective to reduce at least one symptom of the disorder. In some embodiments, an opioid receptor antagonist is an antagonist of a κ-opioid receptor agonist selected from the group consisting of U-50488, U-69593, enadoline, ethylketocyclazocine, salvinorin A and asimadoline. In some embodiments, an opioid receptor antagonist is selected from the group consisting of nor-binaltorphimine, 5′-guanidinonaltrindole, nalmefine, naltrindole, an indolmorphinan, naltrexone and MR2266 ([(−)-(1R,5R,9R)-5,9-diethyl-2-(3-furylmethyl)-2′-hydroxy-6,7-benzomorphan). Related aspects of the invention provide methods for reducing a symptom of such disorders comprising administrations f a therapeutically effective amount of a high molecular weight polyethylene glycol-like compound, such as a polyethylene glycol having an average molecular weight of at least 15 kilodaltons, alone or in combination with the administration of a therapeutically effective amount of a compound described above.

In all of the aspects of the invention drawn to methods of treating a disorder, methods of modulating the activity of PA-I lectin/adhesin, methods of modulating the activity of MvfR, methods of reducing the risk of developing an epithelial cell barrier disorder, or methods of reducing a symptom associated with an epithelial cell barrier disorder disclosed herein, administering the therapeutically or prophylactically active compound, as disclosed herein (e.g., a modulator identified by a screening method according to the invention), is achieved by any mode that is known in the art, and in particular, parenteral delivery, oral delivery, subcutaneous delivery, transcutaneous delivery, intramuscular delivery, intravenous delivery, topical delivery and nasal inhalation. Microbial pathogens, such as P. aeruginosa, not only inhabit the intestinal tract, these pathogens are also capable of Pseudomonas-mediated ophthalmologic, Pseudomonas-mediated otologic and Pseudomonas-mediated cutaneous infection of subjects (e.g., humans). Thus, the invention comprehends administering the active compound by direct routes, such as by topical delivery, cutaneous delivery, intravitreous delivery, and intracerebroventricular delivery, to achieve localized, therapeutically useful concentrations of the compound. In addition, the invention comprehends treatment of any disorder caused, at least in part, by a microbial pathogen such as P. aeruginosa, which includes Pseudomonas-mediated ophthalmologic, Pseudomonas-mediated otologic or Pseudomonas-mediated cutaneous disorders, by administering an active compound through conventional systemic routes, including intravitreously, intracerebroventricularly, and topically (e.g., ophthalmologically, otologically, cutaneously), at levels sufficient to achieve therapeutically useful systemic levels of the active compound. More generally, the invention contemplates any route known in the art to be suitable for preventing or treating such disorder (e.g., infections), including topical, or direct, delivery as well as delivery by a systemic mode of delivering the active compound. A number of administration routes are suitable for either localized administration or systemic administration, for which dosages would be optimized using techniques routine in the art. Still other administration routes include, but are not limited to, intraocular, intrasynovial, transepithelial including transdermal, ophthalmic, sublingual and buccal; topically including ophthalmic, dermal, ocular, rectal and nasal inhalation via insufflation, aerosol and systemic rectal administration.

For each aspect, the invention comprehends the prevention (prophylaxis) or treatment of a disorder selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection.

Another aspect of the invention provides a method of using a κ-opioid receptor antagonist in the preparation of a medicament for treating, ameliorating, or preventing a disorder selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection.

Another aspect of the invention is an isolated modulator identified by an above-described screening method. The invention also comprehends a composition comprising such a modulator and a high molecular weight polyethylene glycol-like compound, such as a polyethylene glycol having an average molecular weight of at least 15 kilodaltons. Further, the invention contemplates an article of manufacture comprising a label packaging material and an effective amount of the above-referenced modulator, wherein the packaging material comprises a label or package insert indicating that the modulator can be used for treating, ameliorating, or preventing an epithelial cell barrier disorder. In some embodiments, the article of manufacture is suitable for treating ameliorating or preventing a disorder selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection. In still another aspect, the invention provides a method of using the modulator described above in the preparation of a medicament for treating, ameliorating, or preventing a disorder selected from the group consisting of gut-derived sepsis, a burn injury, neonatal necrotizing enterocolitis, infection associated with severe neutropenia, toxic colitis, inflammatory bowel disease, irritable bowel syndrome and other GI infectious diseases, enteropathy, transplant rejection, pouchitis, pig belly (i.e., pig-bel), Pseudomonas-mediated ophthalmologic infection, Pseudomonas-mediated otologic infection and Pseudomonas-mediated cutaneous infection.

Other features and advantages of the present invention will be better understood by reference to the following detailed description, including the drawing and the examples.

### BRIEF DESCRIPTION OF THE DRAWING

FIG. 1. PA-I expression is regulated by QS and RpoS. The regulatory region upstream of the leca gene contains both lux box and rpos consensus sequences. The RhlRI system of QS is a key factor in the regulation of PA-I. Diffusible C4-HSL and C12-HSL are self generated by bacteria in response to population density. C4-HSL binds to the dimerized regulator protein RhIR, and then the RhIR-C4-HSL-RhlR complex binds to DNA at the site of lux box upstream of pa-I gene, thereby activating the promoter for RNA polymerase. The promoter of pa-I is specifically recognized by the alternative a σ^{s} factor (Rpos). The binding of the initiation factor σ^{s} to RNA polymerase core enzyme results in the formation of the active form of RNA polymerase that binds to the pa-I promoter initiating the process of transcription. PA-I expression is both QS and RpoS dependent as mutant strains lacking RhIR, RhlI, or RpoS do not produce PA-I.

FIG. 2. Membrane activation of PA-I expression by host cell components. Host cellular elements such as seed extract and cell contact, activate the membrane biosensors CyaB and GacS. These two component transmembrane alarm systems then activate two main global regulators of virulence, Vfr and GacA. Vfr is involved in the activation of LasRI which in turn promotes the activation of the RhIRI system of QS. GacA induces the transcription of lasR and rhlR genes, and is also implicated in the expression of rpoS. Finally a third system PQS, induces expression of both RhlR and RpoS. Thus activation of any of the membrane biosensors could lead to the expression of PA-I with the involvement of a number of different pathways.

FIG. 3. PA-I GFP reporter strain. Plasmid contains QS lux box and RpoS consensus sequences upstream of the PA-I gene.

FIG. 4. PA-I is “in vivo expressed” within the intestinal tract (cecum) of a surgically stressed mouse. PA-I GFP reporter strains were injected into the cecum of mice subjected to sham laparotomy (control) versus 30% surgical hepatectomy (Hep). 24 hours later, fecal preparations and washed cecal mucosa were examined by fluorescent microscopy. Fecal samples were lysed and fluorescence of bacteria quantitated by flourimetry (*P<0.001). RFU=relative fluorescence units of cytosolic preparations of lysed bacteria.

FIG. 5. PA-I and Exo A are increased in the cecum of mice following surgical stress. QRT-PCR of RNA harvested from fresh feces 24 hours following injection of P. aeruginosa into the cecum of mice subjected to sham laparotomy (sham lap) or 30% surgical hepatectomy (Hep) (* P<0.001).

FIG. 6. Soluble factors from the lumen of mouse intestine subjected to segmental ischemia and reperfusion induces fluorescence of PA-I GFP reporter strain. The lumen of the intestine was perfused during ischemia and reperfusion injury. Filtered flushes induced fluorescence. Blood had no effect (*P<0.01). RFU=relative fluorescence units of bacteria lysed of cell membranes.

FIG. 7. Caco-2 cell contact induces PA-I expression via factors within the cell membrane A. The effect of Caco-2 cell fractions on PA-I expression. Only cell membranes induced PA-I expression; an effect that was attenuated in the presence of the GalNAc, a sugar that specifically binds PA-I and prevents bacterial adherence to Caco-2 cells. B. Expression of PA-I mRNA (*P<0.01) following contact to Caco-2 cells. The increase in PA-I mRNA was attenuated in the presence of GalNAc.

FIG. 8. Hypoxia and re-oxygenation (reperfusion) injury to Caco-2 cells activates the fluorescence of PA-I GFP reporter strains via contact dependent and independent mechanisms. A. Reporter strains added to apical wells of Caco-2 cells exposed to hypoxia and normoxic recovery. B. Image of reporter strains displaying contact induced fluorescence at cell-cell junctions. C. Fluorescence of PA-I GFP reporter strains following exposure to apical media only of Caco-2 cells subjected to hypoxia and normoxic recovery. Note delay in onset of fluorescence compared to A. D. Northern blot confirming findings in C (media only) (*P<0.001).

FIG. 9. Distinct protein fractions in the media of Caco-2 cell exposed to hypoxia and hyperthermia (heat shock, stress) enhance the fluorescence in the PA-I GFP reporter strain. A. 10-30 kD media fraction from hypoxic but not heat shock stressed Caco-2 cells, induced fluorescence in the PA-I reporter strain. B. Conversely, a 30-50 kD media fraction from heat shock stressed, but not from hypoxic Caco-2 cells induced fluorescence in PA-I reporter strains. C and D. The effect of both media fractions on the fluorescence of the PA-I reporter strain was markedly attenuated by heat treatment, strongly suggesting that they are proteins.

FIG. 10. Exposure of P. aeruginosa to IFNγ induces PA-I expression and the expression of rhlI. For controls (lane 1, 2) bacteria are suspended in media I (Tryptic soy broth) and media II (DMEM +10% FBS ). Lane 3, 4 are MRNA levels from bacteria exposed to IFNγ and TNFα in DMEM.

FIG. 11. IFN-γ binds to P. aeruginosa in a dose-dependent manner. A. Binding characteristics of recombinant human (rh) IFN-γ to whole bacterial cells (PA27853) by ELISA. B. Alexa 594 labeled antibody staining of IFN-gamma treated PA27853. C. FACS analysis to quantitate % binding of IFN-γ to individual cells of PA27853 (*P<0.001).

FIG. 12. IFN γ binds to solubilized membrane proteins of P. aeruginosa and E. coli A. Binding characteristics of IFN-gamma to solubilized PA27853 membrane fractions by ELISA. B. Solubilized membrane proteins were run on non-denature gels and demonstrated a single immunoreactive band at 30 kD. C. Immunoprecipitation of bacterial membrane proteins showing single immunoreactive band at 25 kD. BSA, Bovine serum albumin.

FIG. 13. IFN-γ enhances barrier dysregulating effect of PA27853 against Caco-2 cells. PA27853 was apically inoculated onto Caco-2 cells in the presence and absence of IFN-γ. Neither media nor IFN-γ alone altered TEER, whereas PA27853 decreased TEER by 60%; an effect which was enhanced in the presence of IFN-γ (* P<0.01) versus PA27853 at 5-hour time point.

FIG. 14. Morphine induces a 4-fold increase in PA-I mRNA in PA27853. Quantitative RT-PCR of PA27853 exposed to morphine (13 μM) and its cognate quorum sensing signaling molecule, C4-HSL (100 μM) (*P<0.001).

FIG. 15. κ and δ opioid receptor agonists induce a 4-fold increase in fluorescence in PA-I reporter strains and enhance the ability of PA27853 to alter epithelial barrier function. A. PA-I GFP reporter strain PA27853/PLL-EGFP was incubated in HDMEM media containing 60 μM of two pure μ opiate agonists (E-1, E-2), a pure κ agonist, and a pure δ agonist. Fluorescence was measured over 9 hours. B. Opiate agonists induced PA27853 to display enhanced barrier dysregulating properties against MDCK cells (*P<0.001). Opioid receptor agonists alone had no effect on monolayer TEER.

FIG. 16. κ and δ opioid receptor agonists increase the production of biofilm in PA27853. Biofilm, a quorum sensing regulated virulence determinant, is markedly increased in PA27853 in the presence of κ and δ opioid receptor agonists. This effect was dose dependent and especially increased with the δ agonist (*P<0.01). Right panel shows actual biofilm assay in presence of varying doses of δ agonist BW373U86.

FIG. 17. The genomically sequenced strain of P. aeruginosa, PA01 makes abundant PA-I protein and decreases the TEER of MDCK cells. The PA01 PA-I transposon mutant knockout PA01/lecA- is void of PA-I protein and does not alter the TEER of MDCK cells. (*P<0.001).

FIG. 18. The PA-I induced decrease in TEER causes apical to basolateral flux of exotoxin A in MDCK cells. Exotoxin A was labeled with Alexa 594 and its transepithelial flux measured at varying levels of TEER in response to PA-I. As TEER decreased below 50% of control, exo A flux increased 5 fold.

FIG. 19. PA-I protein is abundantly expressed in PA01 when strains are exposed to opioid receptor agonists. PA-I protein increased significantly in PA01 in response to opioids. In these experiments the δ agonist, BW373U86, induced PA-I protein to the same degree as C4-HSL.

FIG. 20. Host stress-derived PA-I inducing proteins enhance the ability of PA27853 to decrease the TEER of MDCK cells. PA27853 was apically inoculated onto MDCK cells and TEER examined at the 4-hour time point. Hypoxic media and membranes added to the apical media enhanced the fall in TEER (*P<0.001) versus PA27853. C4-HSL served as a positive control. None of the factors alone changed TER.

FIG. 21. PA-I GFP reporter strains (PA14, PA27853) display enhanced fluorescence in the intestinal tract of the nematode C. elegans. Nematodes were fed strains for 24 hours and imaged. Both strains demonstrate high fluorescence (i.e activation of PA-I) following entry into the digestive tube of worms. No fluorescence was seen in surrounding agar, suggesting that PA-I is activated only within the digestive tube of worms.

FIG. 22. P. aeruginosa strains PA01 and PA14 display differential killing dynamics in C. elegans. Worms were added to agar plates growing lawns of 3 bacterial strains and observed for mortality. OP50 (control), a non-lethal E. coli strain, did not induce any killing, whereas PA-14, a highly lethal strain against C. elegans, induced fast killing (>50% within 18 hours). PAO1 showed intermediate killing dynamics in this model.

FIG. 23. IFN-γ induces the expression of the PA-I lectin in P. aeruginosa. (A) The GFP-PA-I reporter strain, PLL-EGFP/27853, was exposed to media from activated T cells. PA-I expression was assessed in PA-I reporter strains by measuring fluorescence over time and expressed as relative fluorescent units (RFU/control). The PA-I inducing effect of the media from activated T cells (maximum at 7hours) was eliminated by immuno-depletion of the media with anti-IFN-γ antibody. (B) Optical density of P. aeruginosa grown in 96 wells at slow speed showed P. aeruginosa reached stationary growth phase at 6 hours. (C) Only IFN-γ induced PA-I promoter activity after exposure of various cytokines to the GFP-PA-I reporter strain. (D) P. aeruginosa (PAO1) was incubated with 200ng/ml IFN-γ, TNF-α, IL-2, IL-4, IL-8, IL-10 in cell culture media for 4 hours, RNA was extracted, and PA-I mRNA measured by Northern blot. Induction of PA-I mRNA was observed in the presence of IFN-γ and C₄-HSL, but not TNF-α, IL-2, IL-4, IL-8, or IL-10. (E) P. aeruginosa was harvested at 2 hour (OD₆₀₀=1.0) and 4 hour (OD₆₀₀=1.8) after exposure to 200 ng/ml IFN-γ in cell culture media. Northern blot showed PA-I mRNA was significantly increased by IFN-γ exposure at early stationary phase of growth (OD₆₀₀=1.8). (F) Growth phase dependent expression of PA-I lectin measured by immunoblot. PA-I expression was induced following exposure to IFN-γ during stationary phase of growth—an effect not observed during log phase growth. (G) Dose dependent enhancement of PA-I expression following exposure to IFN-γ for 6 hours.

FIG. 24. The presence of rhlI and rhlR, core quorum sensing signaling elements in P. aeruginosa, are required for the PA-I expression and pyocyanin production in response to IFN-γ. (A) P. aeruginosa was harvested at 2 hours (OD₆₀₀=1.0) and 4 hours (OD₆₀₀=1.8) following incubation 200 ng/ml IFN-γ in cell culture media. Northern blot demonstrated that IFN-γ increased rhlI mRNA levels significantly. (B) IFN-γ but not TNF-α induced the transcription of rhlImRNA. (C) The gene product of rhlI, C₄-HSL, a key diffusible quorum sensing signaling molecule, was measured by the luminescence reporter strain pSB536 in PAO1 supernatant and was increased following exposure to IFN-γ. (D) Pyocyanin, an additional quorum sensing dependent virulence factor, was also up-regulated in PAO1 in the presence of 100 ng/ml of IFN-γ. (E) (F) Pyocyanin production required the presence of rhlI and rhlR as mutants did not produce pyocyanin when exposed to IFN-γ. Exposure of the rhiR mutant strain to C₄-HSL did not restore PCN production.

FIG. 25. Identification of the IFN-γ binding site to solubilized membrane fractions of P. aeruginosa (PAO1). (A) Whole cells of P. aeruginosa were coated onto microtiter wells. IFN-γ was added to the wells and binding detected by standard ELISA assay. Dose dependent binding to P. aeruginosa was observed. (B) Epimicrography of immunofluorescence of IFN-γ bound to whole cells of P. aeruginosa. Binding was detected using biotin labeled anti-IFN-γ antibody and fluorescence Alexa 594 labeled streptavidin. Bar: 5 μm. (C) Coating of solubilized membrane protein onto microtiter wells demonstrated that IFN-γ binds to solubilized membrane proteins from P. aeruginosa. (D) Solubilized membrane proteins separated by non-denature PAGE gels were detected using IFN-γ as the first antibody. Representative immunoblot (n=3) demonstrated dose-dependent IFN-γ binding to a single 35 kD solubilized membrane protein. (E) Immunoprecipitation of the solubilized membrane protein with IFN-γ and anti-IFN-γ antibody resulted in a distinctive band. (F) MS/MS spectra of the tryptic peptide (T16) digested protein immunoprecipitated from the solubilized membrane proteins by ESI-TRAP-Electrospray LC-MSMS Ion Trap identified as OprF (outer membrane protein F).

FIG. 26. IFN-γ binds to OprF and induces PA-I expression. (A) ELISA binding assays of IFN-γ to solubilized membrane protein from wild-type P. aeruginosa (PAO1) and the OprF knockout mutant strain 31899 of P. aeruginosa showing attenuated IFN-γ binding to the solubilized membrane protein from the mutant strain. (B) Immunoprecipitation of solubilized membrane proteins with IFN-γ from OprF mutant strain 31899 demonstrating absence of the 35 kD band seen with the parent wild-type strain (PAO1). (C) PA-I protein expression measured by immunoblot in wild-type (PAO1) and mutant strains (31899, 43114) of P. aeruginosa exposed to 200 ng/ml IFN-γ demonstrating an inability of IFN-γ to enhance the expression of PA-I in the OprF mutant strains. (D) Wild-type strain (PAOI) and OprF mutant strains (31899, 43114) carrying the GFP-PA-I fusion plasmid were incubated with 20 ng/ml IFN-γ and fluorescence assessed over time. Results demonstrate a lack of enhanced PA-I expression in mutants exposed to IFN-γ (E) Reconstitution of OprF in mutant strain 31899 demonstrating re-establishment of the responsiveness of PA-I expression to IFN-γ. (F) Anti-OprF antibody (polyclonal: pAb, monoclonal: mAb) was coated onto microtiter plate. The complexes (OprF and IFN-γ, IFN-γ and Lys (lysozyme), OprF and TNF-α) were added and detected by biotin labeled anti-IFN-γ antibody. ELISA assay demonstrated that human IFN-γ binds to purified OprF. Results are a representative experiment of 3 independent studies.

FIG. 27. Epifluorescence photomicrographs of IFN-γ bound to whole cells of P. aeruginosa. P. aeruginosa was incubated with IFN-γ and detected by biotin labeled anti-IFN-γ antibody and alexa 594 labeled streptavidin. Controls included: P. aeruginosa; Streptavidin-alexaS94 only; IFN-γ+Streptavidin-alexaS94; Anti-IFN-γ antibody+streptavidin-alexaS94. Bar: 5 μm.

FIG. 28. Binding characteristics of the IFN-γ to membrane fractions of P. aeruginosa (PAO1). (A) IFN-γ binding capacity to P. aeruginosa harvested at various growth phases were measured by ELISA binding assay. P. aeruginosa at log growth phase has attenuated binding capacity to IFN-γ. (B) Membrane and cytosolic fractions of P. aeruginosa were coated onto microtiter wells. ELISA binding assay demonstrated that IFN-γ preferentially binds to P. aeruginosa membrane fraction. (C) Membrane fractions were digested with proteinase K and retested for IFN-γ binding by ELISA. (D) The binding characteristics of TNF-α, IL-2, IL-4, IL-10, EGF and TGF-β to membrane fractions of P. aeruginosa were determined by ELISA binding assay. Only IFN-γ bound to P. aeruginosa membrane fractions.

FIG. 29. κ-opioid receptor agonists U-50,488 and dynorphin induce PCN biosynthesis in P. aeruginosa 27853 and PAO1. (A) PAO1 grown overnight in tryptic soy broth (TSB) (control) in the presence of 1 mM U-50,488, BW373U86 or morphine demonstrated a bright green appearance in response to U-50,488. (B) PAO1 and 27853 grown overnight in TSB in the presence of varying concentrations of U-50,488 demonstrating increased pyocyanin (PCN) in a concentration-dependent manner. Data are mean±SD (n=3). (C) PAO1 grown overnight in TSB in the presence of varying concentrations of dynorphin demonstrated increased PCN production in a dose-dependent manner. Data are mean±SD (n=5). (D) PAO1 grown overnight in TSB in the presence of 200 μM U-50,488 and varying concentrations of a specific κ-opioid receptor antagonist nor-binaltorphimine (NOR). Data are mean±SD (n=3).

FIG. 30. PCN production dynamics. Dynamic tracking of PCN production in strain PAO1 grown in TSB supplemented with 200 μM of U-50,488 or 200 μM C4-HSL demonstrated a shift in the onset of PCN production at lower cell densities. Data are mean±SD (n=3).

FIG. 31. U-50,488 induces PCN production via elements of QS. (A) Proposed pathway of PCN regulation in P. aeruginosa PAO1. PCN biosynthesis is regulated by the RhIRI system (M. R. Parsek and E. P. Greenberg, Proc Natl Acad Sci U S A 97, 8789 (2000)) which consists of the transcriptional regulator, RhIR, and the synthase RhlI which produces C4-homoserine lactone (C4-HSL). The RhlRI system is regulated by LasRI consisting of the transcriptional activator, LasR, and the synthase LasI which produces 3-oxo-C12-homoserine lactone (30C12-HSL) (T. R. de Kievit, Y. Kakai, J. K. Register, E. C. Pesci and B. H. Iglewski, FEMS Microbiol Lett 212, 101 (2002)). These two core QS systems (LasRI, RhlRI) can be activated by the proximal transcriptional regulator GacA (C. Reimmann, et al., Mol Microbiol 24, 309 (1997)). Another proximal transcriptional regulator MvfR activates 30C12-HSL production (38), and regulates the synthesis of the Pseudomonas quinolone signal (PQS) via expression of the phnAB and pqsABCDE operons controlling the synthesis of the PQS precursors, anthranilate and 4-hydroxy-2-heptylquinoline (HHQ). PQS has also been shown to induce rhlI (S. L. McKnight, B. H. Iglewski and E. C. Pesci, J Bacteriol 182, 2702 (2000)). PQS synthesis is also regulated by LasRI via activation of pqsH- a step that is necessary for the conversion of HHQ to PQS (E. Deziel, et al., Proc Natl Acad Sci U S A 101, 1339 (2004)). (B) Effect of U-50,488 on PCN production in mutants defective in core elements of QS. (C, D) Effect of U-50,488 (C) and C4-HSL (D) on PCN production in mutants defective in the proximal transcriptional activators GacA and MvfR. Data are mean±SD (n=3).

FIG. 32. MvfR plays a key role in the effect of U-50,488 and C4-HSL on PCN production. (A) Complementation of strain 13375 with the mvfr gene restored PCN production at a level above that of the parental strain PAO1 (ΔMvfR, strain 13375 harboring blank plasmid pUCP24; ΔMvfR/mvfR, strain 13375 harboring mvfr gene on pUCP24). (B) Complementation of strain PA06281 with the gacA restored PCN production at a level above that of the parental strain PAO1 (AGacA, strain PA06281 harboring blank plasmid pUCP24; ΔGacA/gacA, strain PA06281 harboring gacA gene on pUCP24). (C) Overnight culture of ΔMvfR/mvfR in TSB showing increased PCN production in the presence of 1 mM of U-50,488 or 100 μM of C4-HSL. (D) AGacA/gacA grown overnight in TSB, showing decreased PCN production in the presence of 1 mM of U20 50,488 or 100 μM of C4-HSL. (E) Dynamic tracking of PCN production in strain AMvfR/mvfR grown in TSB supplemented with 200 μM of U-50,488 or C4-HSL demonstrating a shift in the onset of PCN production similar to that of the parental strain PAO1 (FIG. 30). (F) Dynamic tracking of PCN production in strain in AGacA/gacA grown in TSB supplemented with 200 μM of U-50,488 and C4-HSL demonstrating no shift in the onset of PCN production in the presence of C4-HSL or U-50,488. Data are mean±SD (n=3).

FIG. 33. Intact substrate-binding and DNA-binding domains of MvfR are required for U-50,488- and C4-HSL-inducing effect on PCN production. (A) Specific LysR DNAbinding domain (DBD) from 6 to 64 amino acids (blue) and substrate-binding domain (SBD) from 156 to 293 amino acids (pink) are shown in Mvfr. Truncations of C and N terminus of mvfR produced several versions of truncated MvfR's. (B) C-terminal truncations of 33 (1-299) and 39 (1-293) amino acids do not affect the fuictionality of MvfR as judged by ability of U-50,488 and C4-HSL to induce PCN production. However, C-terminal truncation of 40 (1-292) and additional amino acids (1-291, 1-283) that affected the substrate-binding domain, completely abrogated PCN production and the response to C4-HSL or U-50,488. Similarly, N-terminal truncation of 120 amino acids (121-322) that affected the DNA-binding domain, abrogated PCN production. Data are mean±SD (n=3).

FIG. 34. PQS plays an important role in the pathway by which U-50,488 enhances PCN production in PAO1. (A) Effect of U-50,488 on PCN production in ΔPhnA, a mutant defective in the synthesis of anthranilate, ΔPsqA, a mutant defective in the synthesis of 4-hydroxy-2-heptylquinolone (HHQ), and ΔPhzA1. (B) Use of the compound methyl anthranilate (MA), a known inhibitor of PQS synthesis, attenuated the ability of U-50,488 to enhance PCN production in PAO1. Data are mean±SD (n=3).

FIG. 35. U-50,488 induces biofilm and PA-IL production in P. aeruginosa PAO1 and 27853. (A) Exposure of PAOI to U-50,488 showed a dose dependent increase in biofilm formation. Data are mean±SD (n=5). (B) Dynamic tracking of PA-IL expression by in the GFP-PA-IL reporter strain 27853/ PLL-EGFP (L. Wu, et al., Gastroenterology 126, 488 (2004)) showed a time dependent enhancement of PA-IL expression in response to U-50,488. Data are mean±SD (n=3). (C) Immunoblot analysis of PAO1 exposed to 100 μM of U-50,488 demonstrated an increase in PA-IL protein expression at cell densities of 1.8 (OD600 nm).

FIG. 36. PEG 15-20 attenuates the effect of U-50,488 on PCN production in P. aeruginosa. PAO1 was incubated overnight in TSB (control) or in a 5% PEG 15-20 solution in TSB in the presence of 0.5 mM U-50,488 or 0.2 mM C4-HSL (positive control). PCN was extracted and measured at OD520 nm. Data are mean±SD (n=3).

FIG. 37. There is no FIG. 37 in the application.

FIG. 38. Shows enhancement of PA-I expression, as measured by bacterial fluorescence, in the presence of Caco-2 cells exposed to hypoxia or normoxic recovery. Green fluorescent protein (GFP) reporter strain PA27853/PLL-EGFP was coincubated with Caco-2 monolayers in 96-well fluorimetry plates, following exposure of the epithelia to hypoxia (2 hours at <0.3% O2) and normoxic recovery (hypoxia followed by 2 hours of recovery in normoxic conditions). Although normoxic Caco-2 cells induced a mild increase in fluorescence, GFP reporter strains demonstrated significantly higher PA-I promoter activity within 1 hour of incubation with Caco-2 cells exposed to either hypoxia or normoxic recovery. Data normalized to baseline measurements at time 0 (*P<0.001). RFU, relative fluorescence units.

FIGS. 39. Shows PA-I promoter activity in the presence of hypoxic Caco-2 cells. P. aeruginosa PA-I reporter strains exposed to Caco-2 cells following 90 minutes of normoxic recovery from hypoxia show enhanced fluorescence vs. controls (FIGS. 39A and 39C) and increased numbers of bacteria in the plane of the epithelial cells (FIGS. 39B and 39D), as demonstrated by fluorescence microscopy. FIGS. 39A-B: transmitted light images. FIGS. 39C-D: pseudocolor fluorescence images.

FIG. 40. Shows enhancement of PA-I promoter activity when Pseudomonas is exposed to filtered media from hypoxic and normoxic recovery variants. Filtered media from Caco-2 cells exposed to hypoxia and normoxic recovery were inoculated with the PA-I reporter strain and developed a significant enhancement of fluorescence over controls, although at later time points than those demonstrated when bacteria were able to directly contact epithelial monolayers (*P<0.05, ***P<0.001, data normalized to control). Results were confirmed by Northern blot analysis for expression of PA-I mRNA in wild-type P. aeruginosa. C, control; H, hypoxia; N, normoxic recovery.

FIG. 41. Shows media from the apical chamber of Caco-2 cells is necessary and sufficient for induction of PA-I expression. Isolated media from the basolateral and apical compartments of hypoxic monolayers were added to wells containing the PA-I reporter strain. Only bacteria exposed to apical chamber media displayed an increase in PA-I expression. Hypoxic apical chamber media induced PA-I to a greater degree and at earlier time points than normoxic apical chamber media (*P<0.05, ***P<0.001).

FIG. 42. Shows adenosine exerts a direct effect on PA-I promoter activity in GFP-PA-I reporter strain PLL/EGFP. Dilutions of adenosine in HDMEM were tested against our GFP-PA-I reporter strain. Although PA-I promoter activity was enhanced in response to 10 mM of adenosine (*P<0.05, ***P<0.001), it required at least 6 hours of exposure to the compound, much longer than that observed with hypoxic media alone.

FIG. 43. Shows the change in TER of Caco-2 cells apically inoculated with P. aeruginosa does not vary significantly between hypoxic or normoxic recovery cells and normoxic cells. Transepithelial electrical resistance (TER, or TEER) was measured in Caco-2 cells apically inoculated with PA27853 following exposure to hypoxia and normoxic recovery. Despite the ability of media from hypoxic and reoxygenated Caco-2 cells to increase the expression of PA-I in P. aeruginosa, the TER of Caco-2 cells exposed to these conditions were unchanged in response to a P. aeruginosa challenge. Data normalized to control.

FIG. 44. Shows that hypoxic Caco-2 cells resist the barrier-dysregulating effects of purified PA-I. TER was measured in Caco-2 cells apically inoculated with purified PA-I following exposure to hypoxia. Hypoxic Caco-2 cells apically inoculated with purified PA-I exhibited an attenuated drop in TER compared with normoxic controls. (*P<0.05).

FIG. 45. Shows that Caco-2 cells maintained under hypoxic conditions completely resist the barrier dysregulating effects of apically inoculated P. aeruginosa. TER of Caco-2 cells apically inoculated with wild-type P. aeruginosa in an environment of sustained hypoxia was measured. Caco-2 cells maintained TER equal to hypoxic Caco-2 cells without bacteria and completely resisted the predicted decreased in TER (control=sustained hypoxia; inoculate=sustained hypoxia and inoculation with PA27853). Data normalized to initial value.

FIG. 46. Shows that media from hypoxic Caco-2 cells transferred to untreated Caco-2 cells attenuates the barrier-dyregulating effect of P. aeruginosa. Media of normoxic Caco-2 cells were exchanged with media from hypoxic Caco-2 cells. Epithelial monolayers were then apically inoculated with P. aeruginosa, and barrier function was measured. Normoxic Caco-2 cells exposed to media from hypoxic epithelia displayed a prolonged resistance to barrier dysregulation induced by P. aeruginosa, suggesting that normoxic epithelia may be activated to enhance their barrier function in the presence of soluble mediators produced during hypoxia. (*P<0.05).

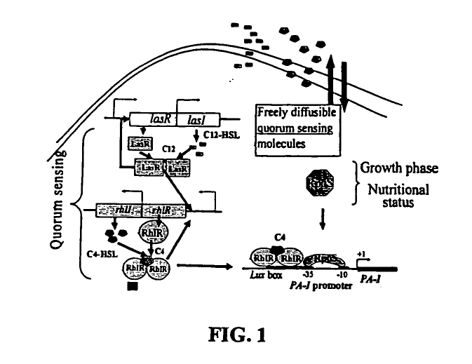

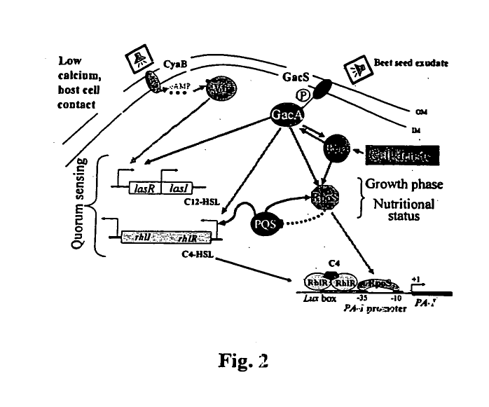

FIGS. 25-1 to 25-22 of Appendix A. The Figures are briefly described in Example 24.

### DETAILED DESCRIPTION

Identification of host stress bacterial signaling compounds and the bacterial membrane receptors to which they bind, will lead to the discovery of therapeutic targets that will allow for interdiction in the infectious process at its most proximate point. Furthermore, the identification of conserved bacterial receptors common to other microbial species will then lead to the development of receptor antagonists or decoys. Such an approach of rendering colonizing pathogens insensate to host stress activators, versus the current approach of elimination therapy with antibiotics, has the potential to provide an ecologically more neutral approach to infection prevention.

The ability of P. aeruginosa to be able to innocuously persist on mucosal surfaces one moment yet switch to a virulent phenotype the next, is one of the many characteristics that defines this highly opportunistic pathogen. In human disease, P. aeruginosa carries among the highest case-fatality rates of any hospital pathogen and is usually associated with patients exposed to prolonged and severe catabolic stress. In fact, among the most important predictors of mortality in patients infected with P. aeruginosa is the length and degree of physiologic stress and injury. The constancy of this observation led to the expectation that compounds released during host stress, especially those that characterize the most critically ill and injured patients, would serve as signals that directly activate the virulence machinery of P. aeruginosa.

While it is generally reasoned that lethal infections caused by opportunistic pathogens such as Pseudomonas are a result of a physiologically weakened host, an alternative explanation is that such pathogens have also evolved mechanisms to directly sense host stress and respond with enhanced virulence. Although it is well established that attachment and invasion of bacteria to host cells results in immediate recognition and immune activation, in the present study we considered the possibility that within this interaction, bacteria in turn, recognize and respond to host immune activation.

The type I-P. aeruginosa lectin (PA-I or leca), an adhesin of P. aeruginosa, was used herein as a representative marker for virulence expression in this organism. The PA-I lectin (7ecA) of P. aeruginosa was known to play a key role in the lethality of this organism in the intestinal tract of a stressed host by creating a permeability defect to its lethal cytotoxins (R. S. Laughlin et al. Ann. Surg. 232, 133 (Jul, 2000)). In addition, the PA-I lectin in P. aeruginosa is “in vivo expressed” within the intestinal tract of mice subjected to physiologic stress in the form of a 30% surgical hepatectomy. PA-I has been shown to also induce apoptosis in respiratory epithelial cells (O. Bajolet-Laudinat et al., Infect Immun 62, 4481 (October 1994)). Expression of PA-I (leca) is regulated by three interconnected systems of virulence gene regulation, the quorum sensing signaling system (QS) (V. E. Wagner, D. Bushnell, L. Passador, A. I. Brooks, B. H. Iglewski, J Bacteriol 185, 2080 (April 2003)), the alternative sigma factor RpoS (K. Winzer et al., J Bacteriol 182, 6401 (November 2000)), and the Pseudomonas quinolone signaling system (PQS) (S. P. Diggle et al., Mol Microbiol 50, 29 (October 2003)).

A model of lethal gut-derived sepsis due to P. aeruginosa has been developed in which mice are subjected to an otherwise recoverable surgical injury (30% hepatectomy) and directly inoculated with P. aeruginosa via the cecum. Mice inoculated with P. aeruginosa who undergo sham surgery fully recover, whereas 90% of surgically injured mice die of sepsis within 48 hours. Using this model, we found that the expression of a quorum sensing (QS)-dependent virulence gene in P. aeruginosa, the PA-I lectin/adhesin (PA-IL or PA-IL lectin), is up-regulated in the cecum of surgically injured mice and can cause a permeability defect in the intestinal epithelium to both elastase and exotoxin A resulting in sepsis and death. Since the quorum sensing signaling system represents an important system of virulence gene regulation in P. aeruginosa, compounds released in response to host injury might signal P. aeruginosa via elements of the quorum sensing signaling system.

The opportunistic pathogen P. aeruginosa is able to sense the presence of the immune cytokine IFN-γ and respond by expressing two quorum sensing dependent virulence factors, PA-I and pyocyanin. From the perspective of P. aeruginosa, the ability to sense and respond to host immune activation, in particular to IFN-γ whose function is directed at bacterial clearance, provides this organism with a countermeasure against host immune activation. In particular, Interferon-γ is shown below to bind to an outer membrane protein in P. aeruginosa, OprF, resulting in the expression of a quorum sensing dependent virulence determinant, the PA-I lectin. These observations provide details of the mechanisms by which prokaryotic organisms are directly signaled by immune activation in their eukaryotic host.

Exposure of P. aeruginosa to opioids leads to the expression of several quorum sensing-dependent virulence factors in P. aeruginosa. That the QS system might be activated by opioids is a significant finding given that QS controls the expression of hundreds of virulence genes in P. aeruginosa (M. Schuster, M. L. Urbanowski and E. P. Greenberg, Proc Natl Acad Sci U S A 101, 15833 (2004)).

Data disclosed herein provide evidence that MvfR is required for PCN production in response to U-50,488. In addition, data from the present study suggest that PCN production in response to U-50,488 also requires the synthesis of Pseudomonas quinolone signal (PQS), since methyl anthranilate attenuated the U-50,488-mediated effect on PCN production. That C4-HSL also requires intact MvfR to produce PCN, coupled with the finding of highly up-regulated PCN production in strains harboring multiple mvJR genes, is consistent with quorum sensing activation relying not only on the binding of QS signaling molecules to their core QS transcriptional regulators ( i.e., RhlR, LasR), but also having QS signals activating proximal transcriptional regulators.

The data disclosed herein establish that opioid compounds may vary in their ability to induce a particular virulence phenotype in P. aeruginosa. It is contemplated that there are multiple host-stress-derived bacterial signaling compounds that are able to influence the state of virulence in P. aeruginosa. Norepinephrine can also affect the QS-dependent virulence factor PA-IL in P. aeruginosa (J. Alverdy, et al., Ann Surg 232, 480 (2000)) and soluble compounds released into the media by hypoxic intestinal epithelial cells also induce PA-IL expression. Consistent with these disclosures is the disclosure that norepinephrine directly affects QS circuitry in E. coli (V. Sperandio, A. G. Torres, B. Jarvis, J. P. Nataro and J. B. Kaper, Proc Natl Acad Sci U S A 100, 8951 (2003)).

Identifying the host stress-derived compounds to which colonizing bacteria respond with enhanced virulence has led to the development of strategies to render bacteria insensate to host stress. The design of specific compounds that interdict in the lines of communication between host factors and the bacterial sensor mechanisms that recognize these factors, has led to strategies which seek to contain rather than eliminate opportunistic pathogens that typically colonize the epithelium of a stressed host (L. Wu, et al., Gastroenterology 126, 488 (2004)).

An understanding of the following more detailed description of the invention is facilitated by establishing the following meanings for terms used in this disclosure, and by a consideration of co-owned International (PCT) Patent Application No. PCT/US02/37498, filed Nov. 26, 2002; provisional U.S. Patent Application No. 60/564,031, filed Apr. 20, 2004. Each of these applications is incorporated herein in its entirety.

An “abnormal condition” is broadly defined to include mammalian diseases, mammalian disorders and any abnormal state of mammalian health that is characterized by an epithelial surface at risk of developing a microbial-mediated disorder. The abnormal conditions characterized by an epithelial surface at risk of developing a microbial-mediated disorder include conditions in which the epithelial surface has developed a microbial-mediated disorder. Exemplary conditions include human diseases and human disorders requiring, or resulting from, medical intervention, such as a burn injury, neonatal enterocolitis, infection associated with severe neutropenia, inflammatory bowel disease, irritable bowel syndrome and other GI infectious diseases, enteropathy (e.g., of the critically ill) and transplant (e.g., organ) rejection.

“Burn injury” means damage to mammalian tissue resulting from exposure of the tissue to heat, for example in the form of an open flame, steam, hot fluid, and a hot surface.

A “chemical contact” injury refers to an injury caused by direct contact with a chemical and can involve a chemical burn or other injury.

“Severe” neutropenia is given its ordinary and accustomed meaning of a marked decrease in the number of circulating neutrophils.

“Transplant rejection” refers to any development of transplanted material (e.g., an organ) recognized as being associated with ultimate rejection of that material by the host organism.

“Administering” is given its ordinary and accustomed meaning of delivery by any suitable means recognized in the art. Exemplary forms of administering include oral delivery, anal delivery, direct puncture or injection, including intravenous, intraperitoneal, intramuscular, subcutaneous, and other forms of injection, topical application, and spray (e.g., nebulizing spray), gel or fluid application to an eye, ear, nose, mouth, anus or urethral opening, and cannulation.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective does. The process of determining an effective dose involves routine optimization procedures that are within the skill in the art.

An “animal” is given its conventional meaning of a non-plant, non-protist living being. A preferred animal is a mammal, such as a human.

In the context of the present disclosure, a “need” is an organismal, organ, tissue, or cellular state that could benefit from administration of an effective dose to an organism characterized by that state. For example, a human at risk of developing gut-derived sepsis, or presenting a symptom thereof, is an organism in need of an effective dose of a product, such as a pharmaceutical composition, according to the present invention.

“Average molecular weight” is given its ordinary and accustomed meaning of the arithmetic mean of the molecular weights of the components (e.g., molecules) of a composition, regardless of the accuracy of the determination of that mean. For example, polyethylene glycol, or PEG, having an average molecular weight of 3.5 kilodaltons may contain PEG molecules of varying molecular weight, provided that the arithmetic mean of those molecular weights is determined to be 3.5 kilodaltons at some level of accuracy, which may reflect an estimate of the arithmetic mean, as would be understood in the art. Analogously, PEG 15-20 means PEG whose molecular weights yield an arithmetic mean between 15 and 20 kilodaltons, with that arithmetic mean subject to the caveats noted above. These PEG molecules include, but are not limited to, simple PEG polymers. For example, a plurality of relatively smaller PEG molecules (e.g., 7,000 to 10,000 daltons) may be joined, optionally with a linker molecule such as a phenol, into a single molecule having a higher average molecular weight (e.g., 15,000 to 20,000 daltons).

“Cell membrane integrity” means the relative absence of functionally significant modifications of a cell membrane as a functional component of a living cell, as would be understood in the art.

“Detectably altered” is given its ordinary and accustomed meaning of a change that is perceivable using detection means suitable under the circumstances, as would be understood in the art.

“Gut-derived sepsis” means the presence of a pathogenic organism or its toxin(s) in the blood or tissues, wherein the organism, e.g., a microorganism, originated in the gut or intestine. In other words, the organism and/or toxin(s) responsible for the septic condition came from the gut or intestine.

“Growth pattern” refers collectively to the values of those properties of a cell, or group of cells (e.g., a population of cells), that are recognized in the art as characterizing cell growth, such as the generation or doubling time of the cell, the appearance of topography of a nascent group of cells, and other variables recognized in the art as contributing to an understanding of the growth pattern of a cell or group of cells.

“Inhibiting” is given its ordinary and accustomed meaning of inhibiting with, reducing or preventing. For example, inhibiting morphological change means that morphological change is made more difficult or prevented entirely.

“PA-I,” or “PA-I lectin/adhesin,” or “PA-IL” expression means the production or generation of an activity characteristic of PA-I lectin/adhesin. Typically, PA-I lectin/adhesin expression involves translation of a PA-I lectin/adhesin-encoding mRNA to yield a PA-I lectin/adhesin polypeptide having at least one activity characteristic of PA-I lectin/adhesin. Optionally, PA-I lectin/adhesin further includes transcription of a PA-I lectin/adhesin-encoding DNA to yield the aforementioned mRNA.

“Epithelium-induced activation” refers to an increase in the activity of a given target (e.g., PA-I lectin/adhesin) through direct or indirect influence of an epithelial cell. In the context of the present invention, for example, epithelium-induced activation of PA-I lectin/adhesin refers to an increase in that polypeptide's activity attributable to the indirect influence of an epithelium manifested through the direct contact of an epithelial cell or cells with an intestinal pathogen.

“Morphological change” is given its ordinary and accustomed meaning of an alteration in form.

“Intestinal pathogen” means a microbial pathogen capable of causing, in whole or part, gut-derived sepsis in an animal such as a human. Intestinal pathogens known in the art are embraced by this definition, including gram negative bacilli such as the Pseudomonads (e.g., Pseudomonas aeruginosa).

“Ameliorating” means reducing the degree or severity of, consistent with its ordinary and accustomed meaning.

“Pathogenic quorum” means aggregation or association of a sufficient number of pathogenic organisms (e.g., P. aeruginosa) to initiate or maintain a quorum sensing signal or communication that a threshold concentration, or number, of organisms (e.g., intestinal pathogens) are present, as would be known in the art.

“Interaction” is given its ordinary and accustomed meaning of interplay, as in the interplay between or among two or more biological products, such as molecules, cells, and the like.

“Transepithelial Electrical Resistance,” or TEER, is given the meaning this phrase has acquired in the art, which refers to a measurement of electrical resistance across epithelial tissue, which is non-exclusively useful in assessing the status of tight junctions between epithelial cells in an epithelial tissue.

“Adherence” is given its ordinary and accustomed meaning of physically associating for longer than a transient period of time.

“Topographically asymmetrical” refers to an image, map or other representation of the surface of a three-dimensional object (e.g., a cell) that is not symmetrical.

“Atomic force microscopy,” also known as scanning force microscopy, is a technique for acquiring a high-resolution topographical map of a substance by having a cantilevered probe traverse the surface of a sample in a raster scan and using highly sensitive means for detecting probe deflections, as would be understood in the art.

“Pharmaceutical composition” means a formulation of compounds suitable for therapeutic administration, to a living animal, such as a human patient. Preferred pharmaceutical compositions according to the invention comprise a solution balanced in viscosity, electrolyte profile and osmolality, comprising an electrolyte, dextran-coated L-glutamine, dextran-coated inulin, lactulase, D-galactose, N-acetyl D-galactosamine and 5-20% PEG (15,000-20,000).

“Adjuvants,” “carriers,” or “diluents” are each given the meanings those terms have acquired in the art. An adjuvant is one or more substances that serve to prolong the immunogenicity of a co-administered immunogen. A carrier is one or more substances that facilitate the manipulation, such as by translocation of a substance being carried. A diluent is one or more substances that reduce the concentration of, or dilute, a given substance exposed to the diluent.

“HMW PEG-like compounds” refer to relatively high molecular weight PEG compounds, defined as having an average molecular weight greater than 3.5 kilodaltons (kD). Preferably, HMW PEG has an average molecular weight greater than 5 kilodaltons and, in particular embodiments, HMW PEG has an average molecular weight at least 8 kilodaltons, more than 12 kilodaltons, at least 15 kilodaltons, and between 15 and 20 kilodaltons. Additionally, “HMW PEG-like compounds includes HMW PEG derivatives wherein each such derivative is an HMW PEG containing at least one additional functional group. Preferred HMW PEG derivatives are cationic polymers. Exemplary functional groups include any of the alkoxy series, preferably C1-C10, any of the aryloxy series, phenyl and substituted phenyl groups. Such functional groups may be attached at any point to an HMW PEG molecule, including at either terminus or in the middle; also included are functional groups, e.g., phenyl and its substituents, that serve to link to smaller PEG molecules or derivative thereof into a single HMW PEG-like compound. Further, the HMW PEG-like molecules having an additional functional group may have one such group or more than one such group; each molecule may also have a mixture of additional functional groups, provided such molecules are useful in stabilizing at least one therapeutic during delivery thereof or in treating, ameliorating or preventing a disease, disorder or condition of an epithelial cell.

“Media” and “medium” are used to refer to cell culture medium and to cell culture media throughout the application. The singular or plural number of the nouns will be apparent from context in each usage.

In general terms, a model of lethal sepsis in mice has been developed which provides unique insight into the process by which microbialpathogens can cause lethal sepsis syndrome from within the intestinal tract of a physiologically stressed host¹⁴. As in human disease, three physiologic “hits” result in mortality, e.g., surgical stress (30% hepatectomy), starvation (48 hour of water only) and the introduction of P. aeruginosa into the distal intestinal tract (cecum). This model results in 100% mortality, whereas elimination of any one of the three factors results in complete survival. A single virulence determinant has been identified in Pseudomonas aeruginosa, PA-I, that is expressed in vivo in response to locally released compounds unique to the intestinal tract of a physiologically stressed host¹⁵. That PA-I plays a role in lethal gut-derived sepsis, such as in mice, was demonstrated by experiments in which mutanized strains of P. aeruginosa, void of PA-I yet capable of secreting exotoxin A, had markedly attenuated effects on the barrier function of cultured epithelial cells and were completely apathogenic in the mouse model of lethal gut-derived sepsis¹⁵. PA-I lectin/adhesin plays a key role in the lethal effect of this organism by creating a permeability defect to potent and lethal cytotoxins of P. aeruginosa, such as exotoxin A and elastase. The lethal effect of intestinal P. aeruginosa appears to occur completely independent of its extraintestinal dissemination (translocation)¹⁵. Surprisingly, systemic injection (intravenous, intraperitoneal) of an equal dose of P. aeruginosa in this model produces no mortality and no systemic inflammation. Taken together, the data provide strong evidence that sepsis can be generated by a microbial pathogen whose virulence is activated locally by host stress-derived BSCs generated during surgical stress.

Observation that P. aeruginosa is much more virulent and lethal when present on an epithelial surface than when bloodbome is supported by a lung model of sepsis¹⁶. Intravenous injection of a highly cytotoxic strain of P. aeruginosa, PA103, resulted in no systemic cytokine release and no mortality in rabbits, whereas lung instillation of an equal dose (approximately 10⁸ cfu/ml) resulted in significant systemic cytokine release (TNFα, IL-8) and 100% mortality. An extensive number of studies have now demonstrated that the most virulent and lethal strains of P. aeruginosa causing sepsis following lung instillation are not those that display the most invasive (translocating/disseminating) phenotype, but rather are those strains that are most disruptive of cellular integrity and epithelial permselectivity to its locally released cytotoxins^{17,18}. These observations, coupled with the findings that P. aeruginosa produces a 25-fold increase in its extracellular virulence factors (i.e., elastase, alkaline protease) when cultured in the presence of epithelial cells¹⁸, suggests that the lethality of this pathogen is governed by its interaction with, and activation by, the epithelium itself. Experimental data show that both soluble and contact-mediated elements of the intestinal epithelium exposed to stress, enhance the capacity of P. aeruginosa to express PA-I, which is capable of causing a profound disruption in the cellular integrity of both intestinal and lung epithelial cells.

The main mechanism of action by which P. aeruginosa induces mortality from within the intestinal tract of a stressed host is via a PA-I-induced permeability defect to its lethal cytotoxins, exotoxin A and elastase¹⁴. Instillation of a combination of purified PA-I with either exotoxin A or elastase into the cecum of surgically stressed and sham-operated control mice induced significant mortality, whereas injection of either compound alone had no effect¹⁴. The clinical role of PA-I was recently examined by screening fecal samples of patients with severe sepsis for whom no source could be identified. Among strains of P. aeruginosa isolated from the feces of critically ill patients, as well as among numerous laboratory and environmental strains, the PA-I genotype has been found to be highly prevalent²⁴. There is now convincing evidence that the intestinal tract environment is a unique niche in which key virulence determinants in highly lethal pathogens (i.e., Vibrio cholera) are activated by yet-unknown “cues” that are present only during active infection²⁵.

The gene encoding PA-I (the lecA gene) is an ideal biological “read-out” and reporter gene in which to examine overall virulence gene expression in P. aeruginosa in response to host stress-derived BSCs.

The precise host cell elements that activate bacterial biosensors are not known. Because PA-I expression is both QS and RpoS dependent, GFP-PA-I reporter strains (described herein) provide a unique opportunity to screen for host cell-derived bacterial signaling compounds released during stress that activate membrane sensors, leading to PA-I expression.

Various opioid receptor agonists, including endogenous morphine alkaloids, are released and maintained at sustained concentrations during severe stress^{50,51}. Opioids are highly conserved compounds and various bacteria and fungi, including P. aeruginosa, synthesize and metabolize morphine^{52,53}. Similarly, as shown herein, elements of the immune system, such as IFN-γ, can also serve as potent host stress-derived BSCs. Although there is data demonstrating that bacteria can activate multiple elements of the host immune system via several bacterial-derived compounds, that such a chemical dialogue functions bidirectionally is virtually unexplored. The ability to expose whole animal models and cell culture systems to clinically relevant stress, while harvesting extracellular fluids and media that can be screened for the presence of host cell-derived bacterial signaling compounds using GPF-PA-I reporter strains, fills a critical gap in knowledge that exists in the area of microbial pathogenesis. Such an approach opens new therapeutic targets for interdiction in the infectious process at its most proximate point.

The invention provides methods of screening for modulators of the signaling induced by one or more BSCs, including such modulators as opioid receptor agonists, morphine, and interferon gamma. These therapeutics are delivered to an organism, such as a human patient, in need thereof. Dosage levels and delivery routes and schedules will vary depending upon circumstances readily identified and accommodated by those skilled in the art using routine procedures.

The therapeutics according to the invention may further comprise a HMW PEG-like compound, which may be administered by any means suitable for the condition or disorder to be treated. The compound(s) may be delivered orally, such as in the form of tablets, capsules, granules, powders, or with liquid formulations including syrups; by sublingual; buccal; or transdermal delivery; by injection or infusion parenterally, subcutaneously, intravenously, intramuscularly, or intrastemally (e.g., as sterile injectable aqueous or non-aqueous solutions or suspensions); nasally, such as by inhalation spray; rectally such as in the form of suppositories; vaginally or urethrally via suppository or infusion, e.g., via cannulation, or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions known in the art.

Exemplary compositions for oral administration include suspensions which may contain, for example, microcrystalline cellulose for imparting bulk, alginic acid or sodium alginate as a suspending agent, methylcellulose as a viscosity enhancer, sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants, such as those known in the art. The inventive compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be excipients such as a relatively high molecular weight cellulose (AVICEL®) or a polyethylene glycol (PEG; GoLytely®, 3.34 kD); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for intestinal administration include solutions or suspensions which may contain, for example, suitable non-toxic diluents or solvents, such as mannitol, 1,3-butanediol, water, Ringer's solution, an isotonic sodium chloride solution, or other suitable dispersing or wetting and suspending agents, including synthetic mono- or diglycerides and fatty acids, including oleic acid. Contemplated in this context are suppositories which may contain, for example, suitable non-irritating excipients, such as cocoa butter, synthetic glyceride esters or polyethylene glycols (e.g., GoLytely®).

The effective amount of a compound of the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like, at risk of developing a microbe-mediated epithelial condition or disease, such as gut-derived sepsis.

The data disclosed herein (see Examples) establish the following:

First, opioids, Interferon-gamma, and media from Caco-2 cells exposed to ischemia and hyperthermia, potently activate the virulence of P. aeruginosa as judged by the expression of PA-I and the ability to shift the barrier dysregulating dynamics of P. aeruginosa against Caco-2 cells.

Second, in clinical models of surgical stress (hepatectomy, ischemia/reperfusion injury to the gut), soluble factors are released into the gut that induce a robust PA-I response in P. aeruginosa.

Third, strains of P. aeruginosa harvested from critically ill humans have a high prevalence of the PA-I genotype/phenotype. (See Ref. 5 Wu in Appendix.) Fourth, strains of P. aeruginosa harvested from critically ill humans display a high level of aggressiveness (adherence, alteration in barrier function, cytotoxicity) to cultured epithelial cells compared to strains harvested from hospitalized non-critically ill patients. (See Ref. 9 Zaborina in Appendix.)

And fifth, high molecular weight polymers (15-20,00 PEG) can render P. aeruginosa completely insensate to host stress-derived bacterial signaling compounds and protect mice from gut-derived sepsis due to this organism. (See Ref. 6 Wu in Appendix.)

### EXAMPLES

### Example 1

Construction of GFP-PA-I Reporter Strains

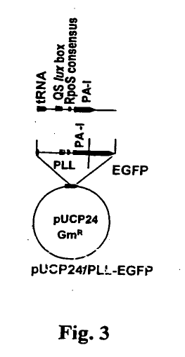

A plasmid containing the GFP-PA-I fusion construct was constructed using conventional recombinant DNA techniques. The EGFP gene encoding green fluorescent protein was amplified using the pBI-EGFP plasmid (Clontech) as a template. XbaI and PstI restriction sites were introduced using primers TCTAGAACTAGTGGATCCCCGCGGATG (SEQ ID NO: 1) and GCAGACTAGGTCGACAAGCTTGATATC (SEQ ID NO: 2). The PCR product was cloned directly into the pCR 2.1 vector using a TA-cloning kit (Invitrogen), followed by transformation of the pCR2.1/EGFP construct into E. coli DH5a. The EGFP gene was excised from this construct by digestion with XbaI and PstI and the fragment containing the excised gene was cloned into the E. coli-P. aeruginosa shuttle vector pUCP24, which had been digested with the same restriction enzymes. The resulting construct (i.e., pUCP24/EGFP), containing the EGFP gene in the shuttle vector, was typically electroporated at 25 μF and 2500 V into P. aeruginosa electrocompetent cells. Cells containing pUCP24/EGFP were selected by gentamicin (Gm)challenge, typically at 100 μg/ml. As illustrated in FIG. 3, a derivative of pUCP24/EGFP was generated that placed the PA-I lectin/adhesin gene in close proximity to the EGFP gene, effectively linking the genes genetically. In addition to incorporating the structural lecA gene, the construct contained the QS lux box and RpoS consensus sequences in the 5′ non-coding region of lecA, along with rRNA sequence. The derivative construct was termed pUCP24/PLL-EGFP.

Upon consideration of FIG. 3, in conjunction with the explanatory text provided herein, one of skill would understand how to make and use the above-described construct, as well as other suitable constructs for providing lecA, alone or in physical proximity to a marker gene such as EGFP, using any of a variety of techniques.

### Example 2

Location of PA-I

PA-I lectin/adhesin was localized to a previously undescribed structural appendage on the outer surface of P. aeruginosa, using conventional techniques as would be understood in the art.

### Example 3

Correlation of In Vitro and In Vivo Observations

C. elegans is suitable as an in vivo model system for BSC signaling and its role in the production of PA-I. C. elegans is accepted as a highly accurate and predictable model in which to study the host response to P. aeruginosa^{54 55}. C. elegans worms feed on lawns of P. aeruginosa growing on solid agar and, thus, provides an ideal system in which to study microbial pathogenesis, especially in regard to gut-derived sepsis, since the mode of infectivity is via the digestive tract. These nematodes readily feed on bacteria such as E. coli growing on solid agar plates, yet when fed specific strains of P. aeruginosa, mortality rates exceed 50% within 72 hours. Mortality rates with this model have been shown to be dependent on both the agar environment as well as the strain of P. aeruginosa. Certain strains are highly lethal in this model (e.g., PA14), whereas other strains (PAO1) show intermediate kill rates⁵⁶. The ability to feed C. elegans on lawns of the completely sequenced P. aeruginosa strain PAO1, and selected transposon mutants, while enriching agar plates with various host stress-derived BSCs screened for their ability to express PA-I, makes available a rapid screening system for genes that actively participate in in vivo virulence against the intestinal epithelium. With this approach, the virulence phenotype observed in vitro has been transferred to an in vivo model, with the expectation that results obtained with such a model will prove much more reliable in accurately characterizing the virulence phenotype observed in human patients suffering from an epithelial cell barrier dysfunction.

### Example 4

In Vitro Recapitulation of the In Vivo “cues” Released During Surgical Stress

In vitro studies demonstrated that pH, osmolality, and norepinephrine did not change PA-I expression, while opioids, interferon-gamma, C4-HSL, and media from hypoxic and hyperthermic intestinal epithelial cells induced PA-I expression. PA-I was functionally expressed in epithelial cell assays in the presence of the PA-I-inducing compounds.

### Example 5

Toxin Flux Across Epithelia

Exotoxin A was labeled with AlexaFluor 594, and its transepithelial flux was measured at varying levels of decrease of transepithelial resistance (TEER) of MDCK monolayers that was achieved by apical application of MDCK cells to different concentrations of pure PA-I protein. A five-fold increase in exotoxin A flux across MDCK cells was found when transepithelial resistance was decreased below 50% of control. Purified PA-I decreased the TEER of epithelial cells to the same degree as P. aeruginosa. PA-I null mutants of P. aeruginosa had a significantly attenuated effect on the transepithelial resistance of MDCK cells. Techniques used in conducting the experiments are described in Example 23, below, or are conventional in the art.

### Example 6

Response of Epithelia to Purified PA-I

The degree of cell polarity (i.e., degree of cell confluency and tight junctional apposition) has been shown to dictate the degree of response to purified PA-I protein. Cells that were loosely confluent had a more profound fall in TEER in response to PA-I compared to “tighter” and more differentiated cell monolayers. In addition, wounded monolayers exposed dense areas of PA-I binding. Cell culturing was performed as described in Example 23, below; relative confluency was assessed using conventional techniques as would be known in the art.

### Example 7

Soluble Host Factors Induce Expression of PA-I Lectin/Adhesin

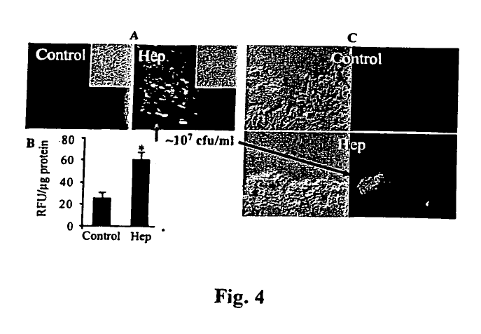

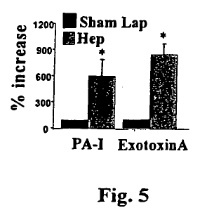

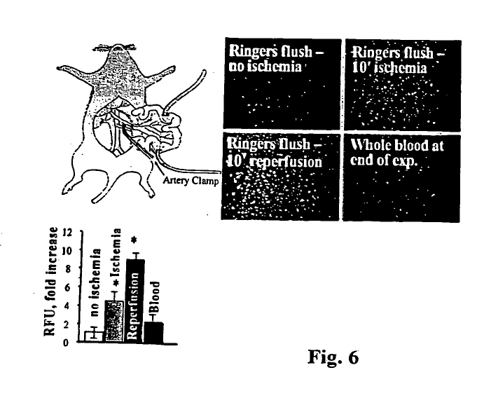

GFP-reporter strains permit demonstrations that virulence gene expression in P. aeruginosa is expressed in vivo within the intestinal tract of a stressed (30% hepatectomy) host. EGPF reporter constructs were specifically designed to contain known upstream regulatory regions involved in PA-I expression (e.g., lux box (QS promoter elements) and RpoS; see FIG. 3). The EGFP-PA-I reporter strain, termed PLL-EGFP, was then injected into the cecum of sham-operated (control) mice and mice undergoing surgical hepatectomy. Twenty-four hours later, feces and washed cecal mucosa were then assayed for the presence of fluorescent bacteria. As can be seen in FIG. 4, both within the cecal lumen and in response to contact with the intestinal epithelium, PA-I is expressed in vivo in response to elements of the local intestinal micoenvironment (cecum) of mice subjected to catabolic (surgical) stress. These findings were verified in the non-reporter strain, PA27853, using an assay in which bacterial RNA is extracted from fresh feces using a RNA protection system. Reiterative studies were performed in which PA27853 was introduced into the cecum of control and hepatectomized mice and then bacterial RNA recovered from fresh feces 24 hours later for quantitative RT-PCR (QRT-PCR) of both PA-I and exotoxin A. This assay provides a precise molecular “snapshot” of the effect of the in situ cecal environment on P. aeruginosa virulence gene expression. Results demonstrated that the cecal microenvironment of a stressed host induced PA-I and exotoxin A virulence gene expression (FIG. 5). Next, in order to determine whether these findings were due to soluble factors released into the intestinal lumen, particulate-free filtrates were prepared from cecal luminal contents from control and hepatectomized mice and added to fresh cultures of the reporter strain PLL-EGFP. Results demonstrated that when PA-I GFP reporter strains were exposed to filtered cecal contents from mice exposed to surgical hepatectomy, a 248%±12 increase in fluorescence was observed compared to 112%±15 for filtered cecal contents from sham-operated mice (P<0.001). These results indicated that a soluble factor is present in the intestinal lumen following surgical stress that activates PA-I expression. Two remaining issues included, first, whether the soluble PA-I-inducing components are generated from within the intestinal tract itself or from the systemic compartment and, second, whether the soluble PA-I-inducing components are specific to hepatectomy-induced stress. To address these issues an animal model of segmental intestinal ischemia was developed in which an isolated loop of intestine (6 cm, proximal ileum) was luminally cannulated and timed aliquots of luminal perfusates were collected following 10 minutes of ischemia followed by 10 minutes of reperfusion. Blood was then obtained at the end of the experiment in order to determine the effect of systemic factors on PA-I expression. Results seen in FIG. 6 suggested that 1) intestinal ischemia, similar to hepatectomy, can release soluble factors into the intestinal lumen capable of signaling P. aeruginosa to express PA-I; 2) these factors may originate from the intestinal tract itself, since during ischemia the intestine is isolated from systemic factors; 3) blood components do not induce PA-I expression; and 4) the presence of the normal flora, virtually absent in flushed small bowel segments, appears to play no role in this response. To rule out the possibility that the in vivo expression of PA-I was due to secondary effects of surgical stress on physico-chemical changes in the local microenvironment, P. aeruginosa strain PA-27853 and reporter strains (PLL-EGFP) were exposed to ambient hypoxia (0.3% O₂), pH changes (6-8), and 80% CO₂. None of these conditions induced PA-I expression. In addition, reporter strains exposed to the blood or liver tissue of mice following sham-operation or hepatectomy, did not display enhanced fluorescence. These studies suggest that bacterial signaling components released in response to surgical and ischemic stress are highly concentrated in the intestinal tract and are generated by host-cell derived factors that can be isolated from, and detected within, the intestinal lumen. Based on these results, it is expected that any form of stress (e.g., surgery, injury such as traumatic injury, illness, heat, starvation, hypoxia, and the like) to epithelial cells, such as intestinal epithelial cells, will typically lead to a change in the level of at least one soluble factor involved in bacterial signaling, i.e., at least one soluble BSC.

### Example 8

Bacterial Signaling Compounds (BSCs) inducing PA-I Lectin/Adhesin Expression are Found in Epithelial Cells

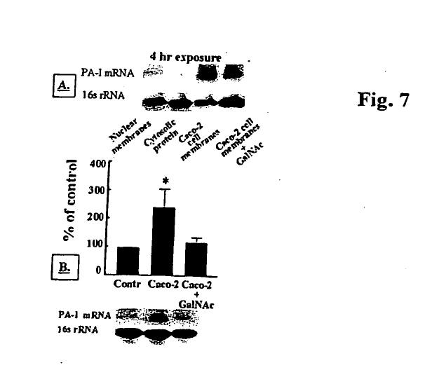

Using Caco-2 intestinal epithelial cells, the issue of whether components of intestinal epithelial cells themselves played a role in triggering the expression of PA-I was addressed. Strain PA27853 was exposed to media (apical and basolateral) and Caco-2 cell fractions (cytosolic, nuclear, membrane) at various time intervals. PA-I mRNA was measured in PA27853 in response to the various Caco-2 cell media fractions in the presence and absence of GalNac, a sugar that binds specifically to PA-I and prevents P. aeruginosa adherence to Caco-2 cells. Media alone from Caco-2 cells grown in transwells (apical or basolateral) had no effect on PA-I expression. However, Caco-2 cell membrane fractions triggered the accumulation of a very high abundance of PA-I mRNA (>10 fold increase)-an effect that was attenuated in the presence of GalNac (FIG. 7A). These in vitro findings are in agreement with the above mouse studies showing that PA-I can be activated in response to contact with the intestinal epithelium, yet in the unstressed Caco-2 cell system, luminal contents (apical media) had no effect, similar to the control mice. Experiments in which PA27853 were inoculated onto the apical surface of Caco-2 cells and allowed to densely adhere (extended culture), demonstrated an increase in PA-I mRNA, which was nearly completely abolished in the presence of GalNac (FIG. 7B). Thus, PA-I expression is influenced by both membrane-bound and soluble factors, and it is contemplated that modulators of the bacterial signaling process include, but are not limited to, effectors (i.e., enhancers, activators, and inhibitors) of a soluble factor, a membrane-bound factor, or both.

### Example 9

Stressed Caco-2 Cells Release Soluble Factors that Induce PA-I Lectin/Adhesin Expression

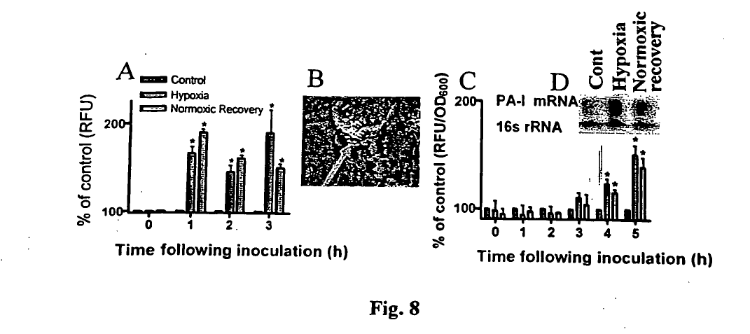

In order to recapitulate the type of stress that the intestinal epithelium is exposed to under conditions of surgical injury, a confluent monolayer of Caco-2 cells was subjected to hypoxic stress (1 hour 0.3% hypoxia+30 minutes normoxic recovery). A PA-I GFP reporter strain, PLL-EGFP, was then exposed to the apical media from stressed and non-stressed cells. Results in FIG. 8 demonstrate a rapid and significant increase in PA-I promoter activity in these strains based on relative fluorescence units (RFU's) of PLL-EGFP. Results were confirmed by Northern blot analysis. Analysis of the spatial and temporal dynamics of these experiments was carried out using fluorescent microscopy. In hypoxic cells, contact-induced expression of PA-I promoter activity was observed and demonstrated preferential adherence of bacteria to the tri-cellular junctions of Caco-2 cells (FIG. 8B). Reiterative experiments exposing Caco-2 cells to heat shock stress (42° C. 1 hour+2 hours recovery) demonstrated similar findings to hypoxia. A near ten-fold increase in fluorescence was observed in the PA-I GFP reporter strain exposed to apical media from heat shock stressed Caco-2 cells. Membrane fractions from both hypoxic and heat shock stressed Caco-2 cells induced extremely high PA-I expression (approximately 100 fold) that could not be quantifiably distinguished between groups.

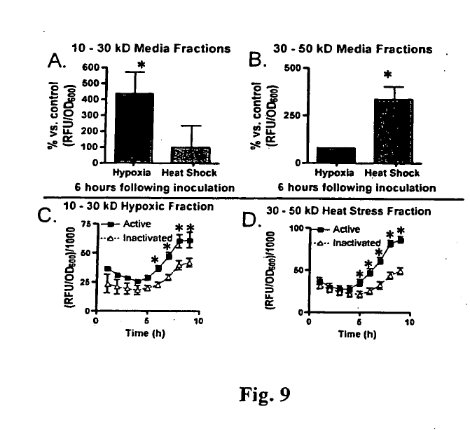

Media from hypoxic and heat shock stressed Caco-2 cells were next fractionated into 5 molecular weight fractions (<3, 3-10, 10-20, 20-30, >30 kDa) using centricones, to determine if a specific MW fraction could be identified that induces PA-I expression. In addition, to determine if the bacterial signaling compound(s) was a protein, fractions were treated with heat inactivation and the protein inhibitor, proteinase K. Results are summarized in FIG. 9. Data from these experiments strongly suggest that there are two distinct bacterial signaling compounds released into the apical media in response to hypoxic and heat shock stress in Caco-2 cells that are proteins (peptides) (FIGS. 9C, D). These findings are significant because 1) the fractionated compounds are soluble and can be mass produced in unlimited supply by growing large sheets of Caco-2 cells, and 2) the compounds are proteins and therefore can be easily characterized by mass spectrometry and identified. Although more highly purified and characterized factors will facilitate technological development, screens for modulators of the activity (e.g., bacterial signaling activity) of such factors are presently available, with variations on a given screening methodology apparent to one of ordinary skill using no more than routine procedures.

Stimulated Immune Cells Release Factors that Induce PA-I Lectin/Adhesin Expression

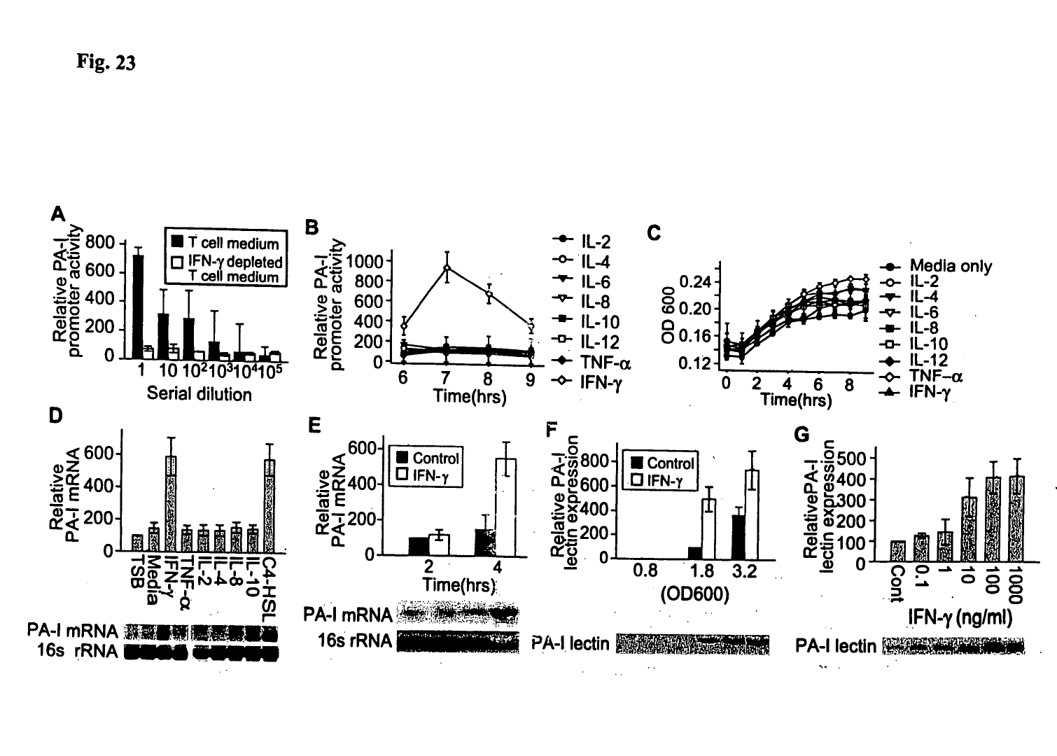

Immune elements released at the mucosal epithelial surface, the primary site of colonization for P. aeruginosa, were considered to be suitable candidates to serve as host stress-derived bacterial signaling compounds. As a physiologically relevant in vitro system to determine whether immune factors can activate P. aeruginosa virulence, supernatants from antigen-stimulated T cells were evaluated for their ability to increase PA-I expression in the P. aeruginosa strain PLL-EGFP/27853, which carries a PA-I-GFP reporter construct. P. aeruginosa cells were incubated with supernatants from stimulated T-cells and PA-I expression was assessed by GFP expression levels (fluorescence). Media from activated T cells, which release a comprehensive array of cytokines (D. J. Schwartzentruber, S. L. Topalian, M. Mancini, S. A. Rosenberg, J Immunol 146, 3674 (May 15, 1991)), induced PA-I expression as assessed by enhancement of fluorescence in the PA-I-GFP fusion reporter strain (L. Wu et al., Gastroenterology 126, 488 (February 2004)) (FIG. 23A).

To determine whether this effect was due to cytokines, the reporter strain was exposed to various cytokines (human IL-2, IL-4, IL-6, IL-8, IL-10, IL-1 2, Interferon gamma (IFN-γ) and tumor necrosis factor alpha (TNF-α) with only IFN-γ showing a significant increase in PA-I expression beginning at early stationary phase of growth (FIG. 23C). None of the cytokines tested had any significant effect on bacterial growth (FIG. 23B). To test whether IFN-γ was required in the media of activated T-cells to enhance PA-I expression, we depleted IFN-γ from the culture media of activated T cells using specific antibody. Immunodepletion of the media of IFN-γ resulted in the complete loss of its PA-I inducing capacity (FIG. 23A), suggesting that IFN-γ is essential for PA-I expression in this system. To further confirm the role of IFN-γ as a host stress derived bacterial signaling compound, we exposed the completely genomically sequenced strain of P. aeruginosa, PAO1 (C. K. Stover et al., Nature 406, 959 (Aug. 31, 2000)), to human recombinant IFN-γ, TNF-α, and various other cytokines (IL-2, IL-4, IL-8, IL-10) and measured lecA (encoding for PA-I) mRNA by Northern blot. IFN-γ, but not TNF-α or other cytokines, induced lecA mRNA (FIG. 23D). These data indicated that human IFN-γ functions as a host cell-derived bacterial signaling molecule to which P. aeruginosa responds with enhanced virulence.

### Example 10

Identification of Host Stress-Derived BSCs by screening Candidate Agents: the Role of Cytokines

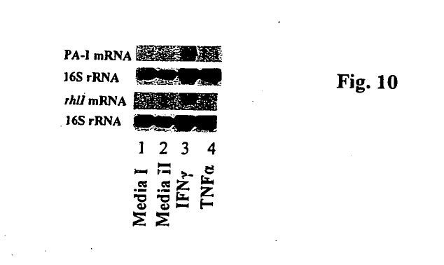

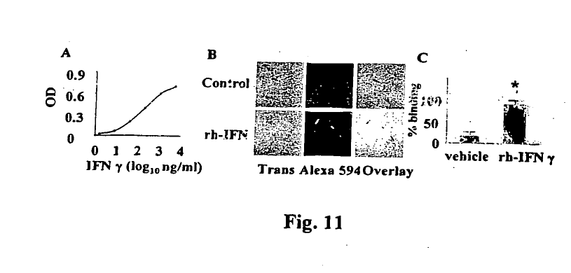

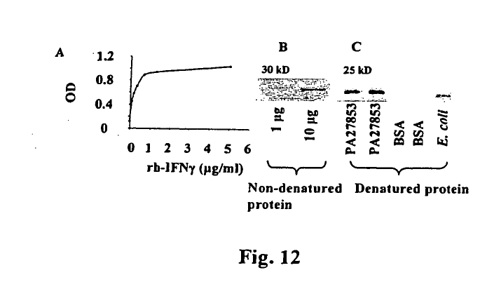

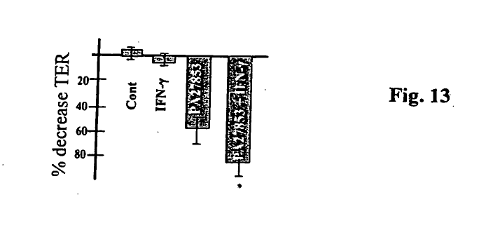

As a method to rapidly identify host BSCs, P. aeruginosa strains were exposed to media containing adenosine (released by Caco-2 cells in response to hypoxia) TNFα, IL-2, IL-6 IL-8 (released by epithelia in response to bacterial invasion/ischemia), and IFNγ (released by intraepithelial lymphocytes in response to bacterial invasion/ischemia). In addition, strains were exposed to apical media from Caco-2 cells basolaterally exposed to single and combinations of the various epithelial-derived cytokines. Dr. Jerrold Turner, has demonstrated that basolateral exposure of Caco-2 cells to the combination of IFN γ and TNF α activates cellular signaling proteins that dramatically alter tight junctional proteins and function⁶³. Media from Caco-2 cells exposed to various combinations of these cytokines had no effect on PA-I expression. However, IFN-γ alone induced a direct effect on PA-I expression while none of the other compounds alone had any effect (FIG. 10). Another issue was whether IFN γ binding to P. aeruginosa could be demonstrated for strain PA27853. Using both ELISA, immunofluorescence microscopy, and flow cytometry, the binding characteristics of IFNγ were determined for both whole bacteria and membrane fractions of P. aeruginosa. Results demonstrated that IFN-γ showed high binding affinity to whole bacterial cells of PA27853 (FIG. 11). These effects were also observed with strain PAO1. Next, solubilized and separated membrane proteins of P. aeruginosa (PA27853) were solubilized and separated, which showed that IFN-γ avidly binds to a single 30 kDa protein band (FIG. 12). It has been difficult to immunoprecipitate a significant quantity of this protein from PA27853, but it has been determined that this protein can also be immunoprecipitated from E. coli (FIG. 12C). Next, IFN-γ binding specificity, to whole bacterial cells, was determined, using reiterative binding studies in the presence of various gram negatie bacterial strains, including P. aeruginosa. Multiple strains of bacteria displayed IFN-γ binding by ELISA binding assays suggesting that an IFN-γ binding site may be conserved across a wide variety of procaryotic cells. Finally, in order to determine if PA-I was functionally expressed in PA27853 in the presence IFN-γ, PA27853 was inoculated onto Caco-2 cell monolayers in the presence of IFN-γ and the effect on barrier dysregulating dynamics of PA27853 against this cell line were assessed to determine if IFN-γ shifted the dynamics. Results seen in FIG. 13 demonstrate that IFN-γ enhanced the barrier dysregulating effect of PA27853 against the intestinal epithelium after five hours of incubation. Thus, cytokines such as IFN-γ are embraced by the invention as effective modulators of bacterial signaling and, ultimately, of eukaryotic (e.g., epithelial) cell barrier function.

The expression of virulence in P. aeruginosa is highly regulated by the quorum sensing signaling system (QS), a hierarchical system of virulence gene regulation that is dependent on bacterial cell density and hence growth phase (M. Whiteley, K. M. Lee, E. P. Greenberg, Proc Natl Acad Sci USA 96, 13904 (Nov. 23, 1999)) (S. P. Diggle, K. Winzer, A. Lazdunski, P. Williams, M. Camara, J Bacteriol 184, 2576 (May, 2002)). Therefore in order to determine the effect of growth phase on the response of P. aeruginosa to IFN-γ, bacteria were harvested at various growth phases following exposure to IFN-γ, and PA-I mRNA and protein measured by Northern blot and immunoblot respectively. Both PA-I mRNA and protein were increased in response to IFN-γ at early stationary phase of growth (FIG. 23E, 23F). PA-I protein expression in PAO1 was also dose dependent, with the greatest increase seen with 100 ng/ml (FIG. 23G). Taken together these results suggested the exposure of P. aeruginosa to IFN-γ enhanced PA-I expression but was not able to shift its expression to an earlier phase of growth.

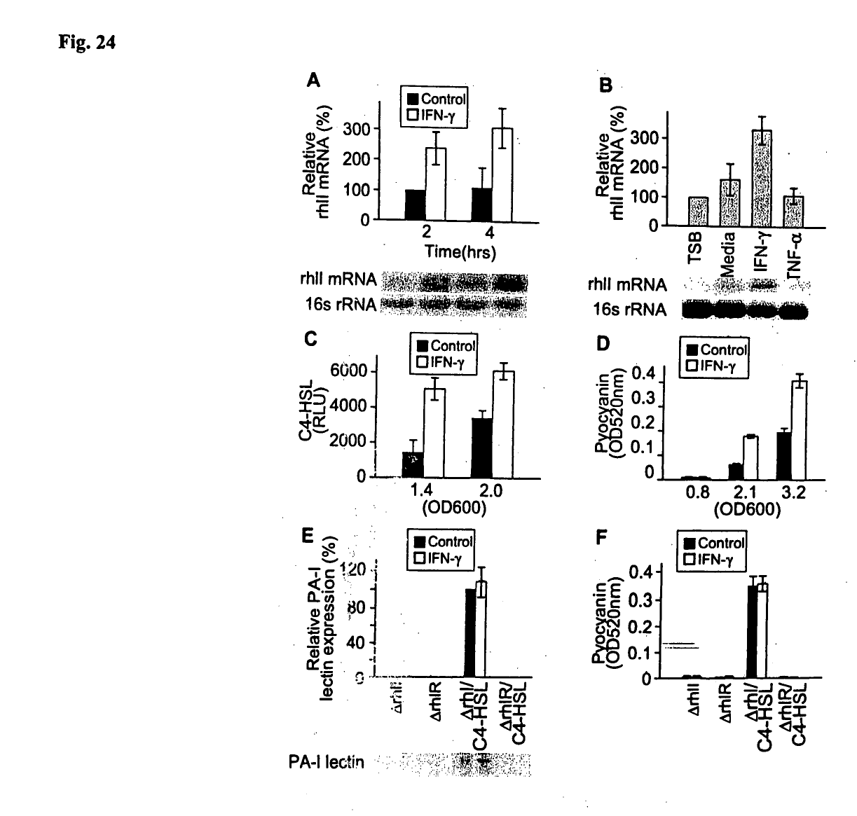

To determine whether IFN-γ induced PA-I via activation of the quorum sensing signaling system, we measured rhilI gene expression in PAO1 in response to IFN-γ by Northern blot. IFN-γ induced rhli transcription in PAO1 (FIG. 24A, 24B). RhlI is the gene required for the synthesis of C₄-HSL (C₄-homoserine lactone), a core quorum sensing signaling molecule that plays a key role in the expression of PA-I (M. R. Parsek, E. P. Greenberg, Proc Natl Acad Sci (USA) 97, 8789 (Aug. 1, 2000)). We next determined if exposure of P. aeruginosa to IFN-γ would lead to the synthesis of C₄-HSL. PAO1 was exposed to 100 ng/ml of IFN-γ and C₄-HSL measured in bacterial supernatants. C₄-HSL synthesis was increased in PAO1 exposed to IFN-γ (FIG. 24C). To verify that activation of the QS system by IFN-γ led to the production of other QS-dependent virulence products, we measured pyocyanin production, a redox active compound, in PAO1 at various phases of growth following exposure to IFN-γ and showed that IFN-γ increased pyocyanin production in PAO1 (FIG. 24D). Finally, to determine whether rhli and rhlR are required for the production of pyocyanin (PCN) and PA-I expression in response to IFN-γ, an rhlI mutant P. aeruginosa strain and, independently, an rhIR⁻ mutant P. aeruginosa strain were exposed to IFN-γ. PCN production and PA-I expression induced by IFN-γ were abolished in these mutant strains (FIGS. 24E, 24F). These data suggest that the QS system plays a key role in the response of P. aeruginosa to IFN-γ.

### Example 11

Interferon-γ Binds to the Surface of P. aeruginosa

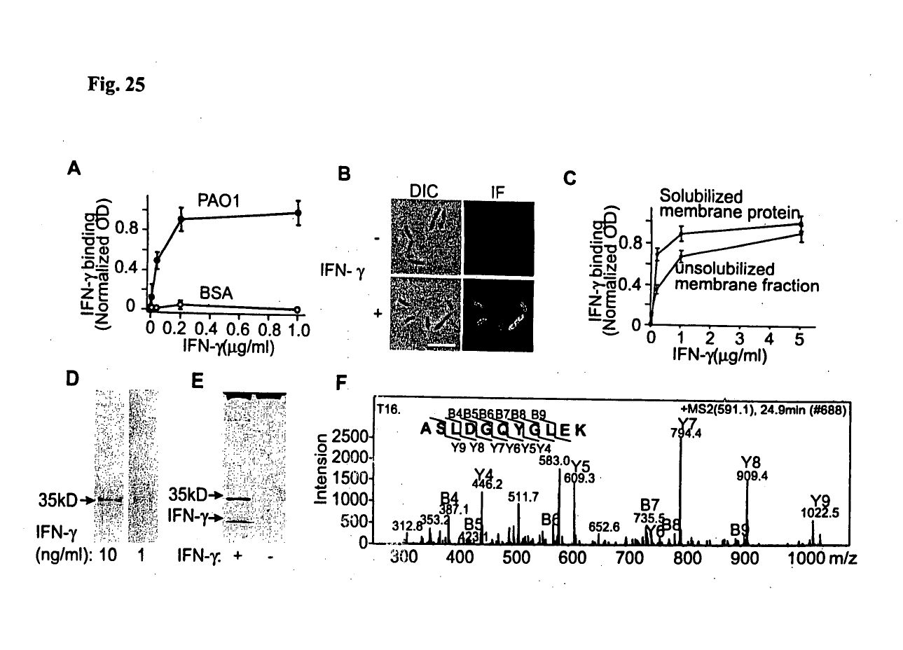

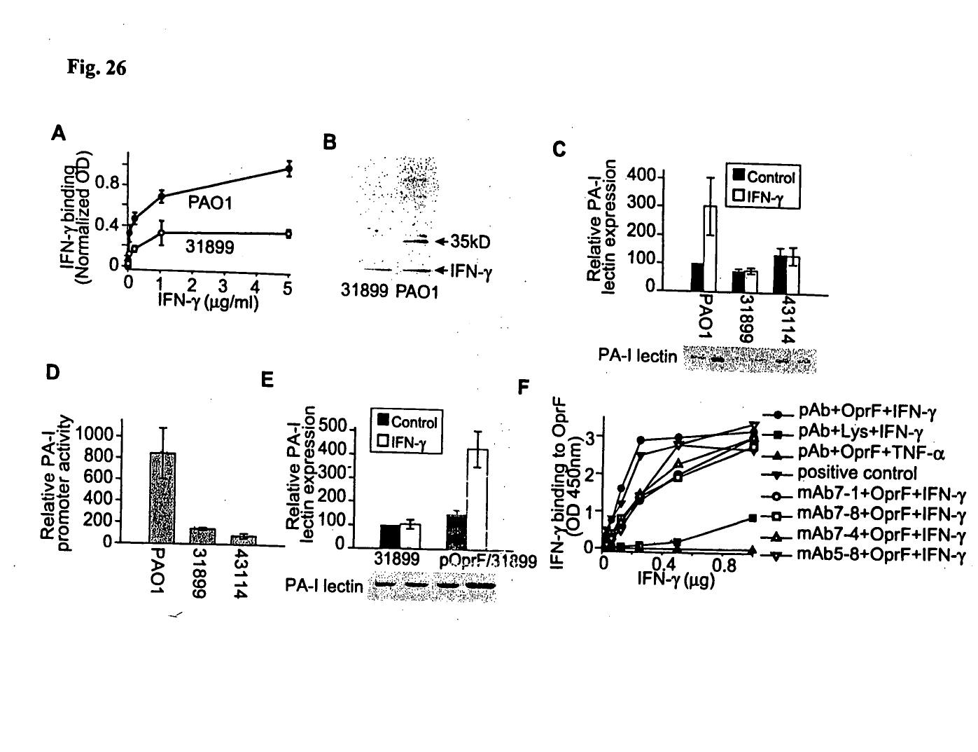

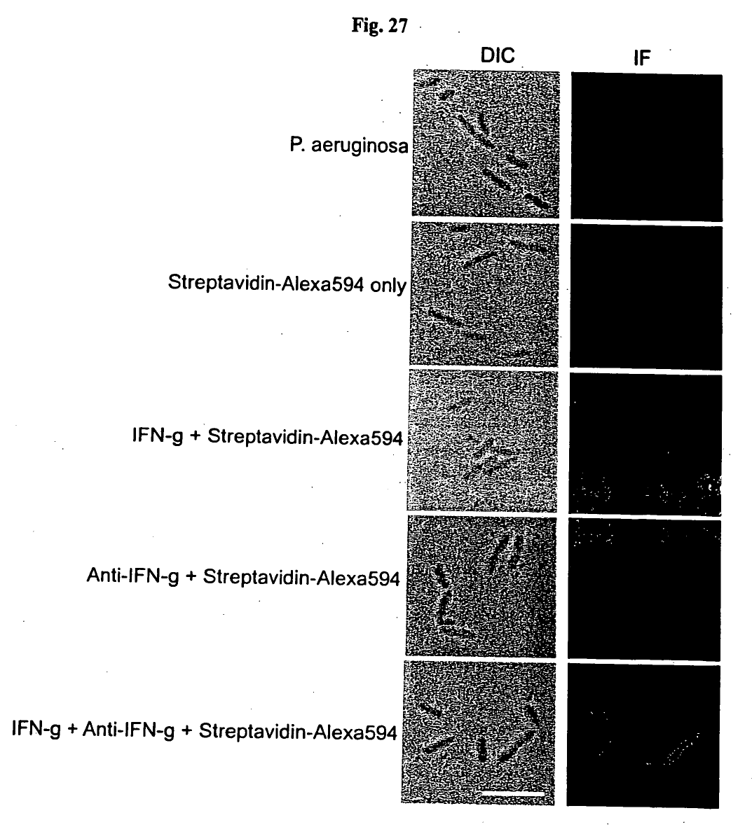

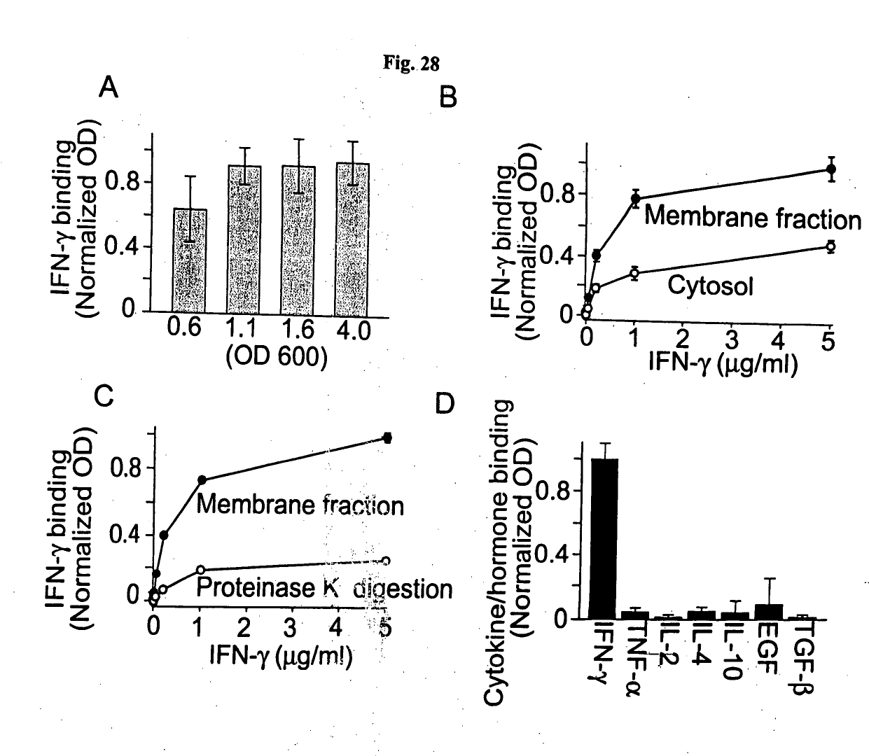

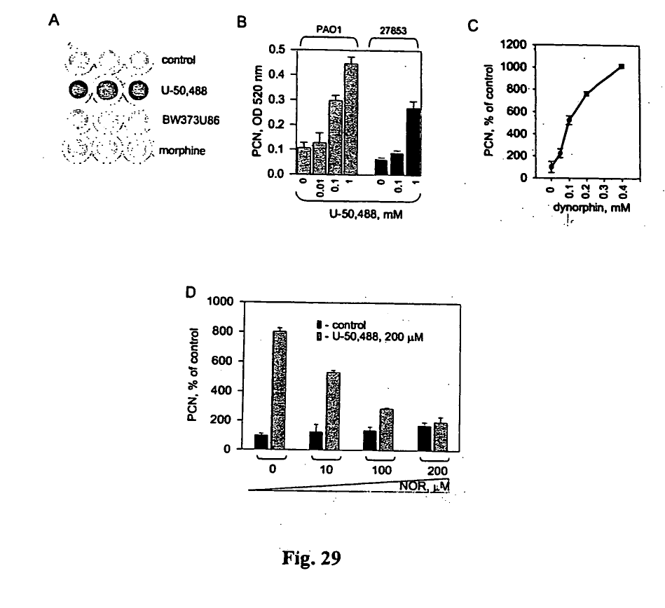

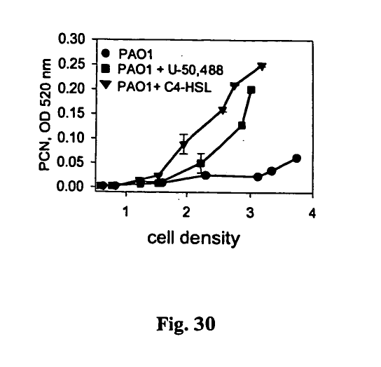

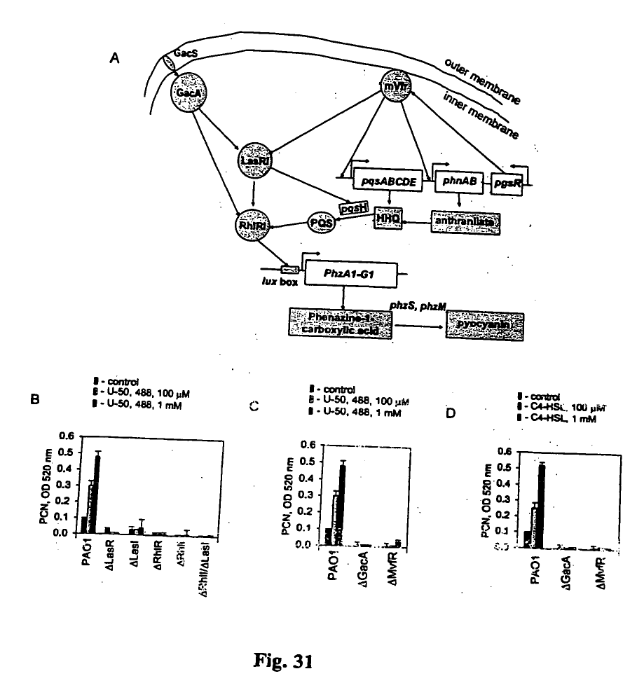

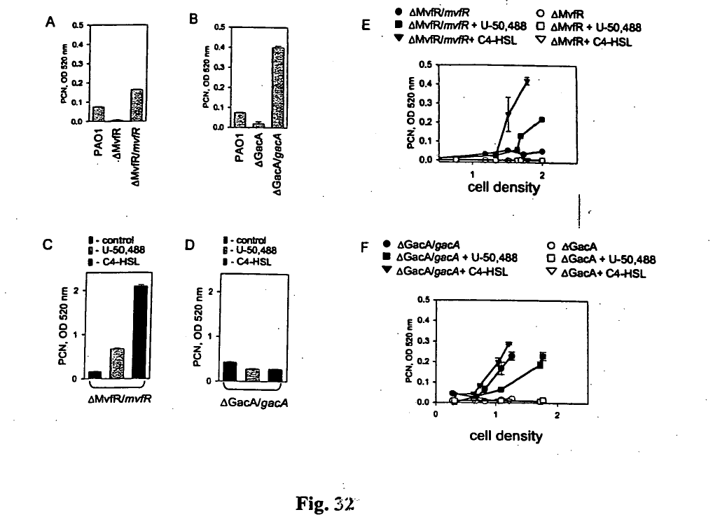

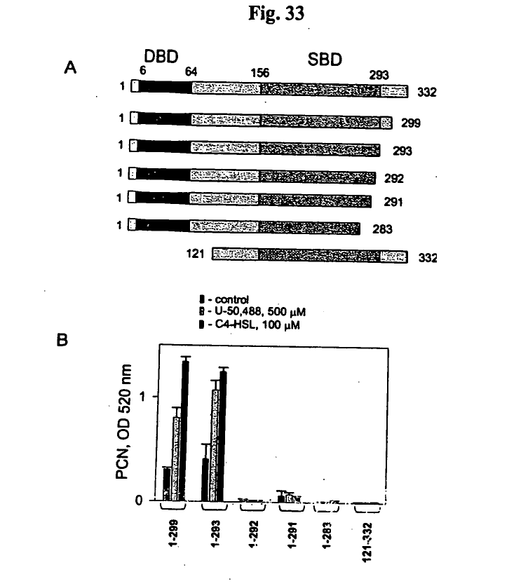

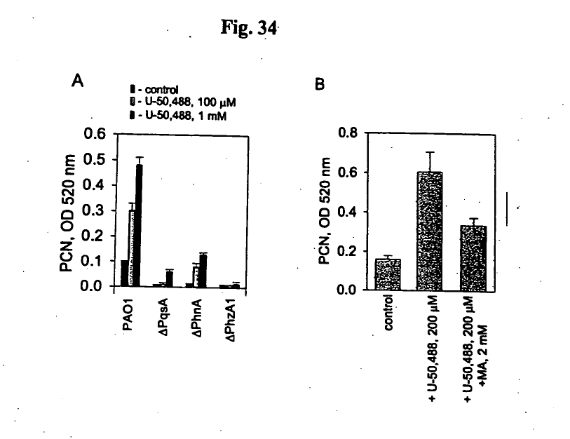

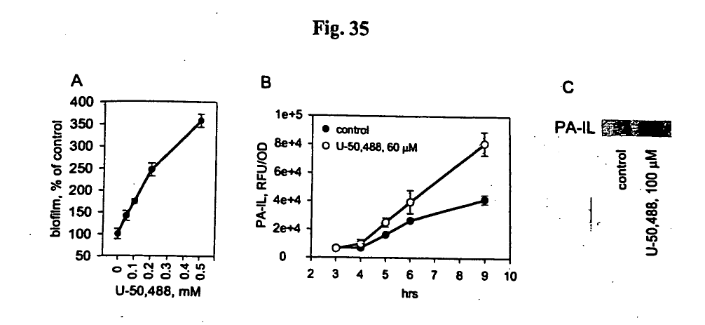

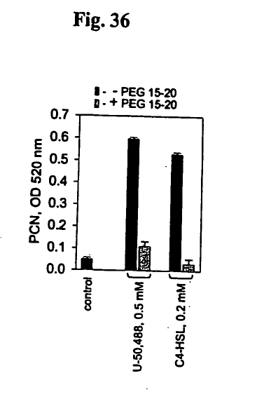

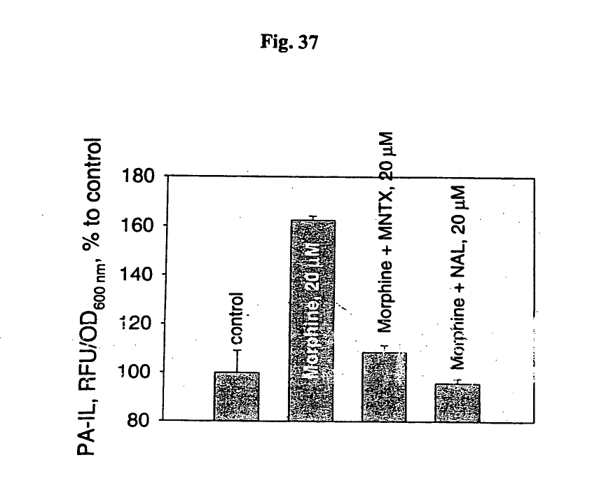

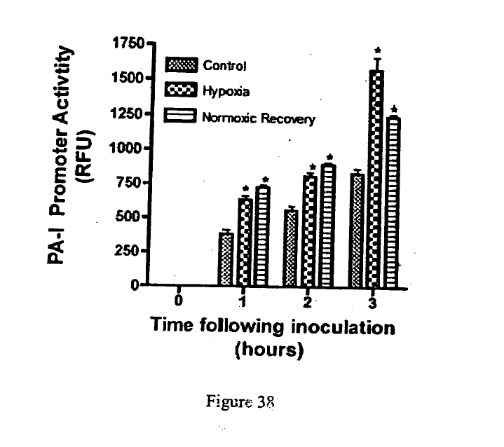

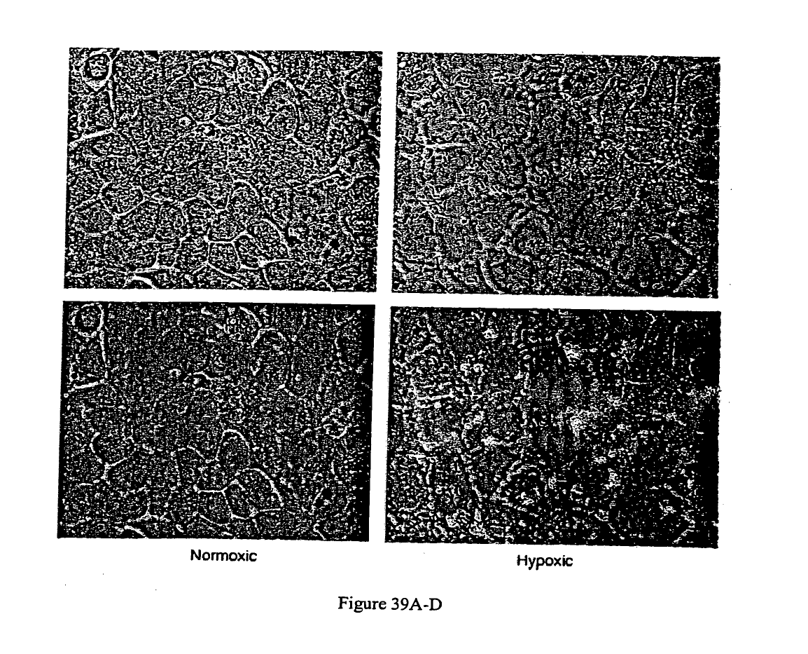

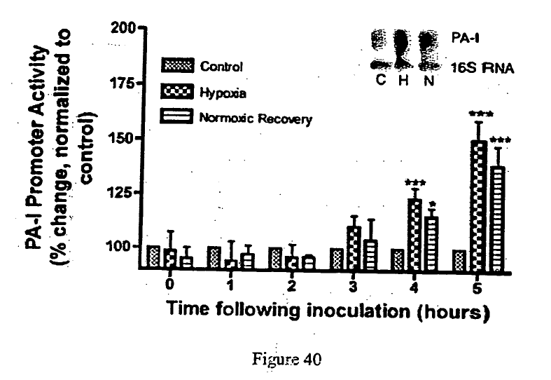

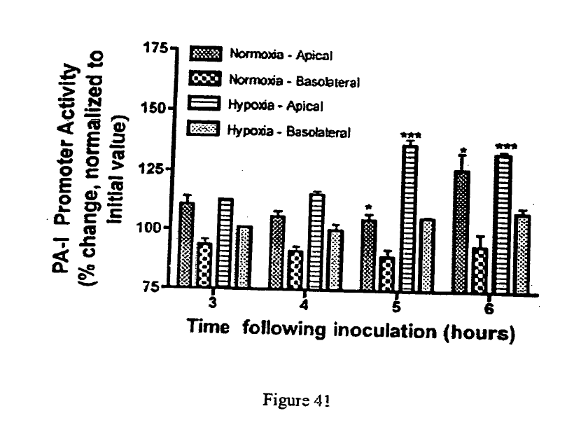

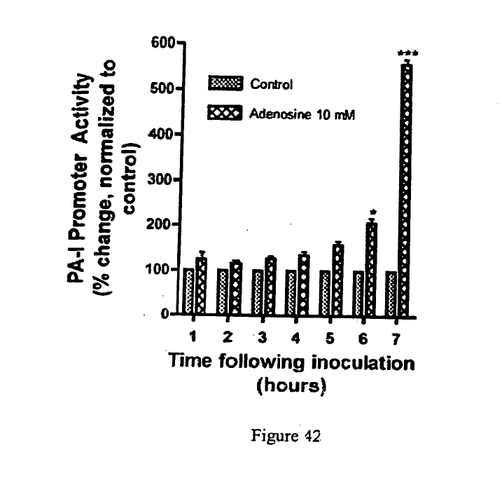

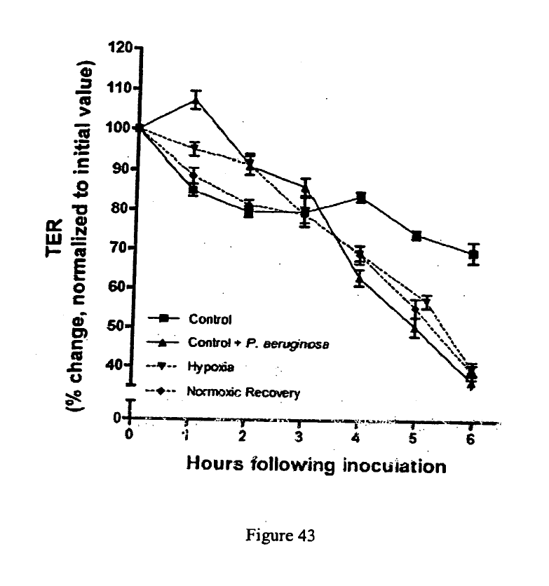

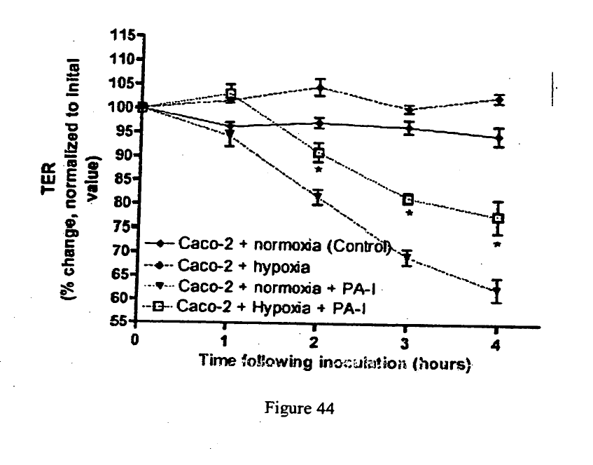

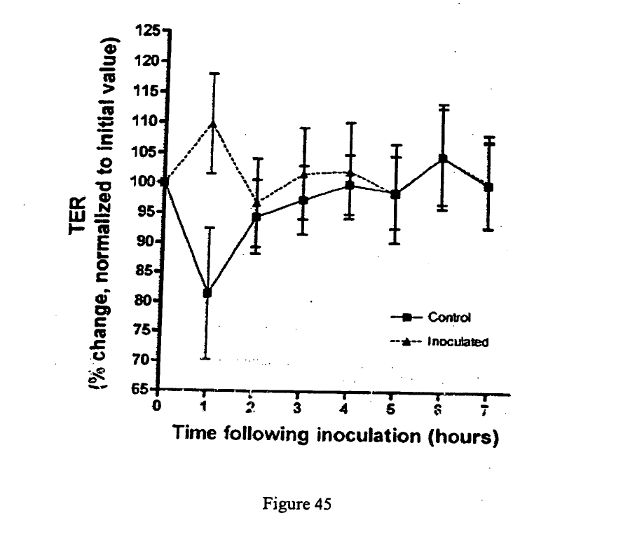

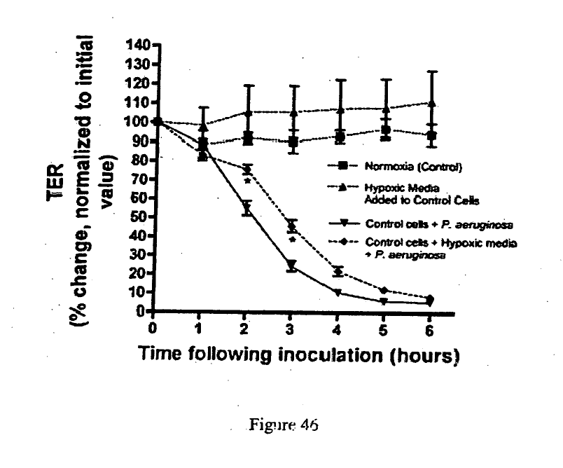

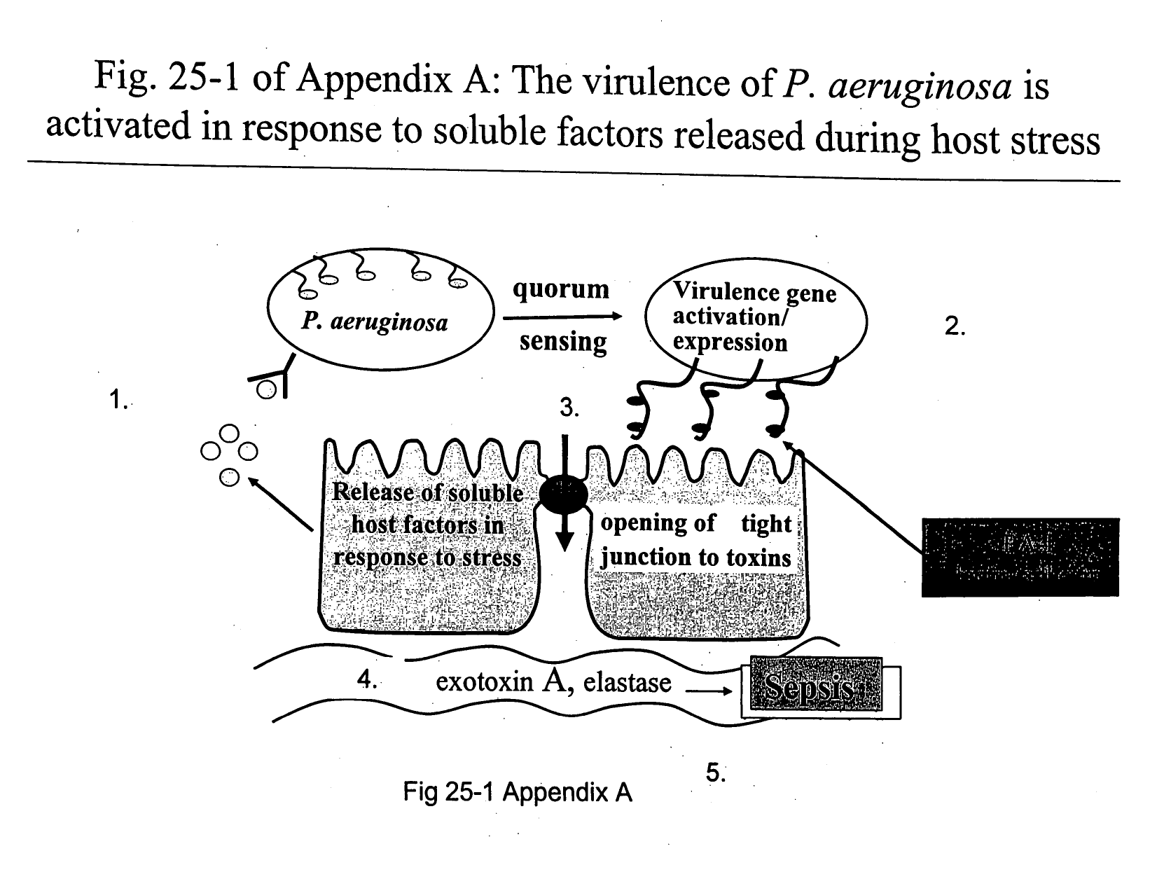

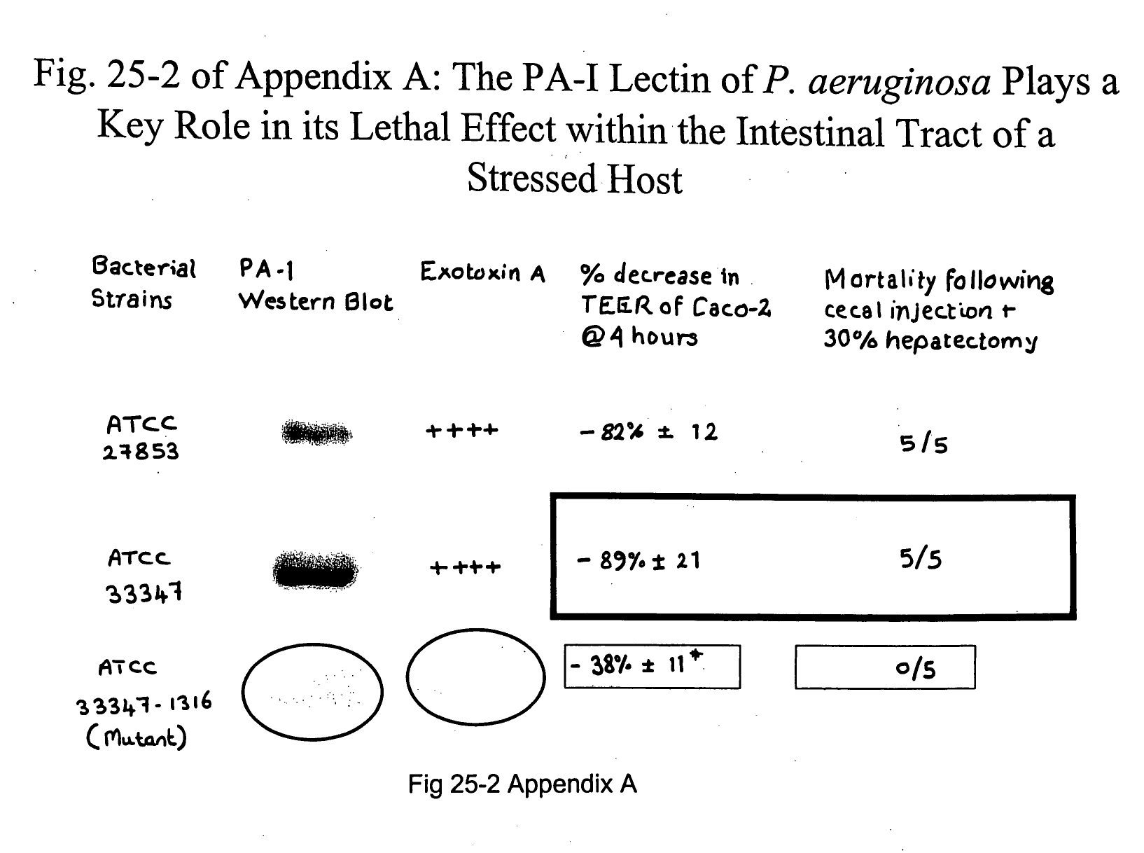

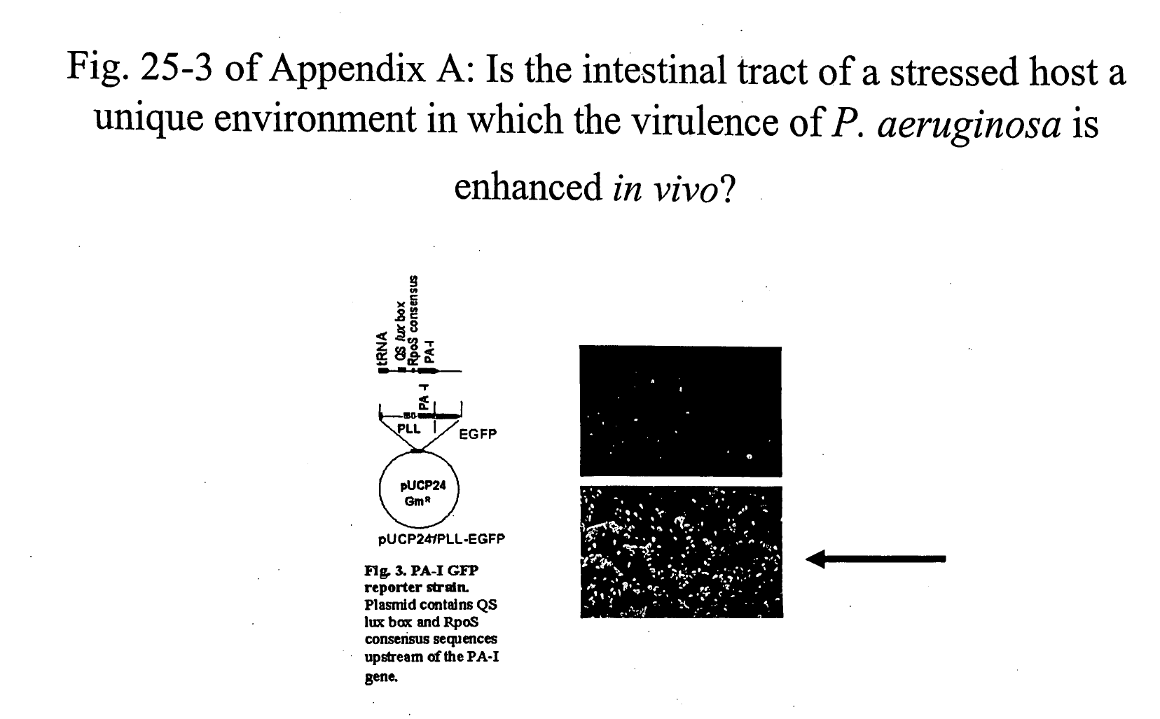

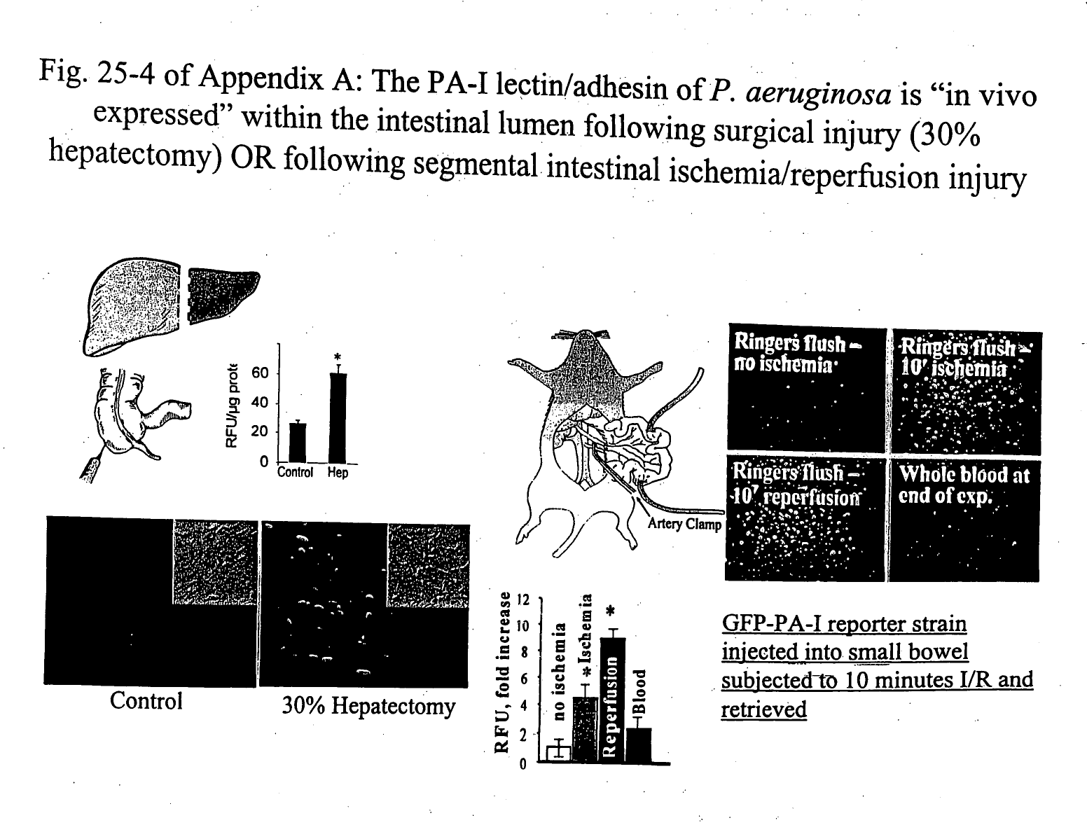

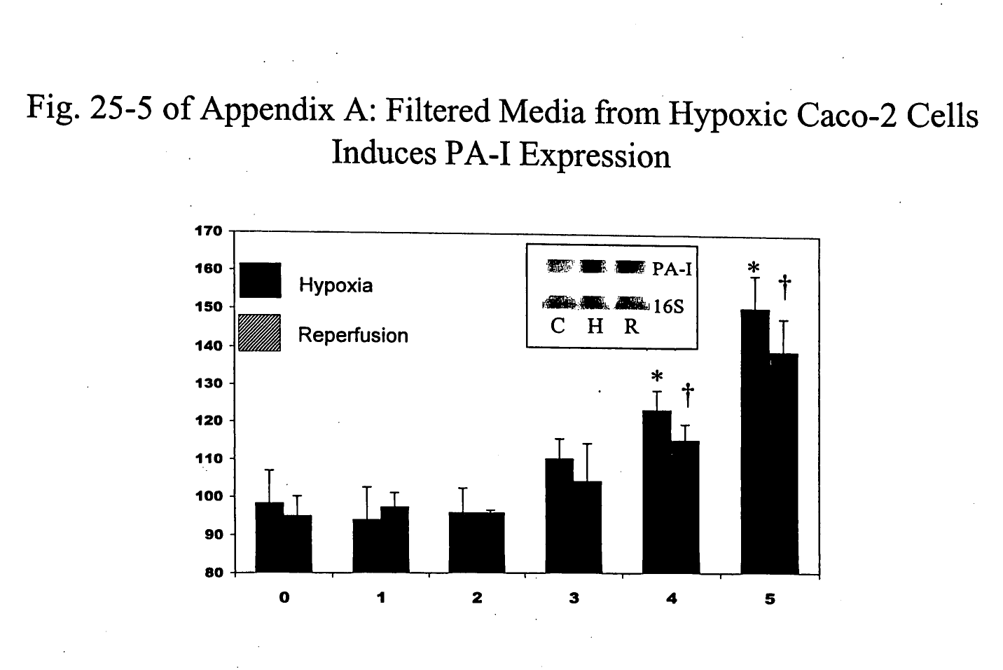

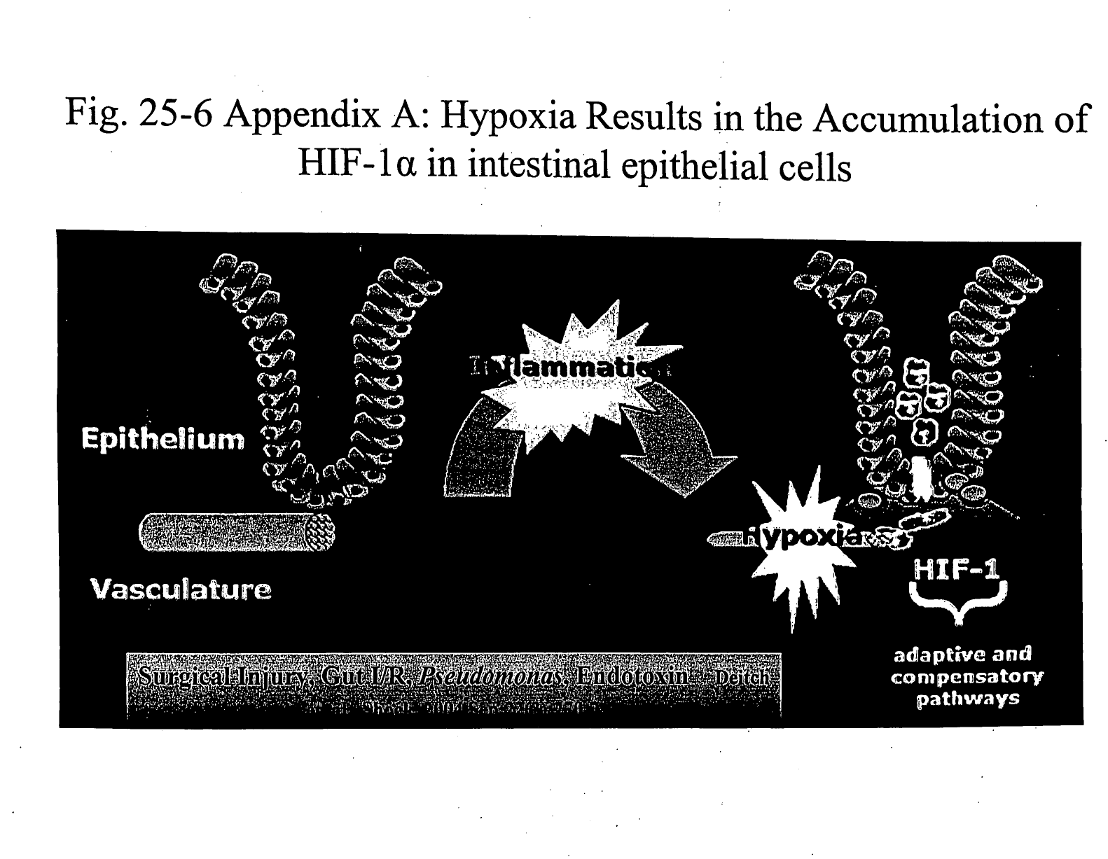

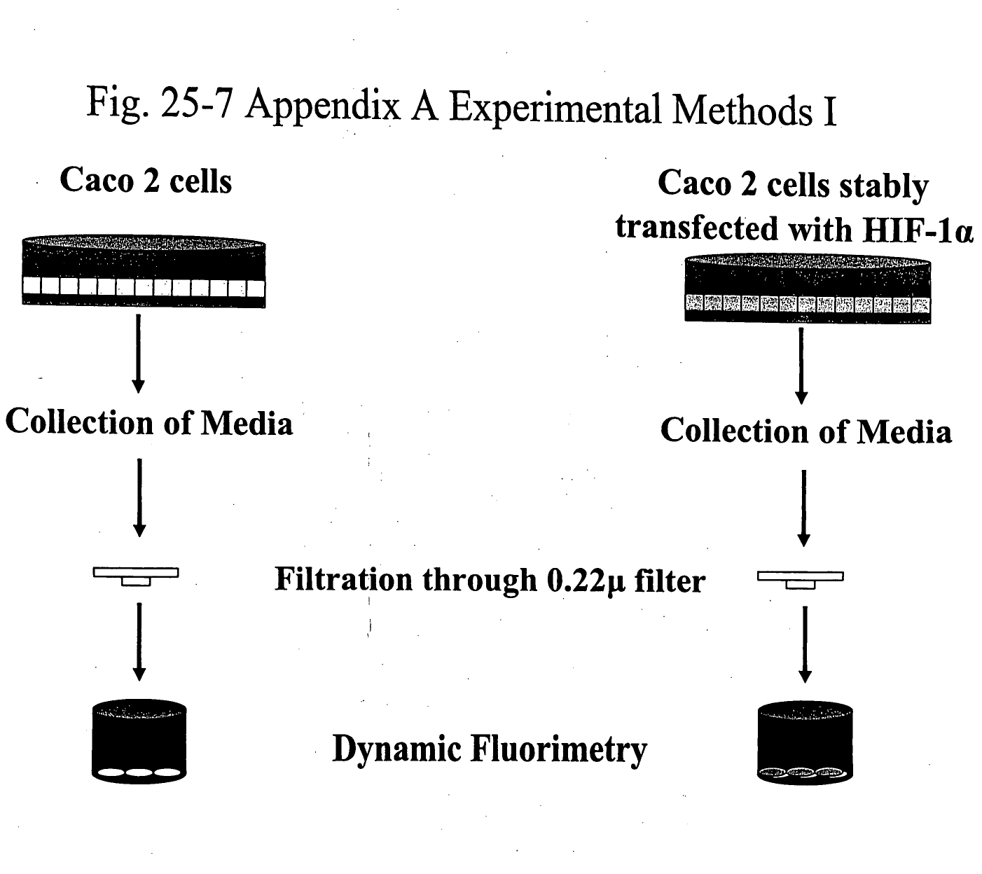

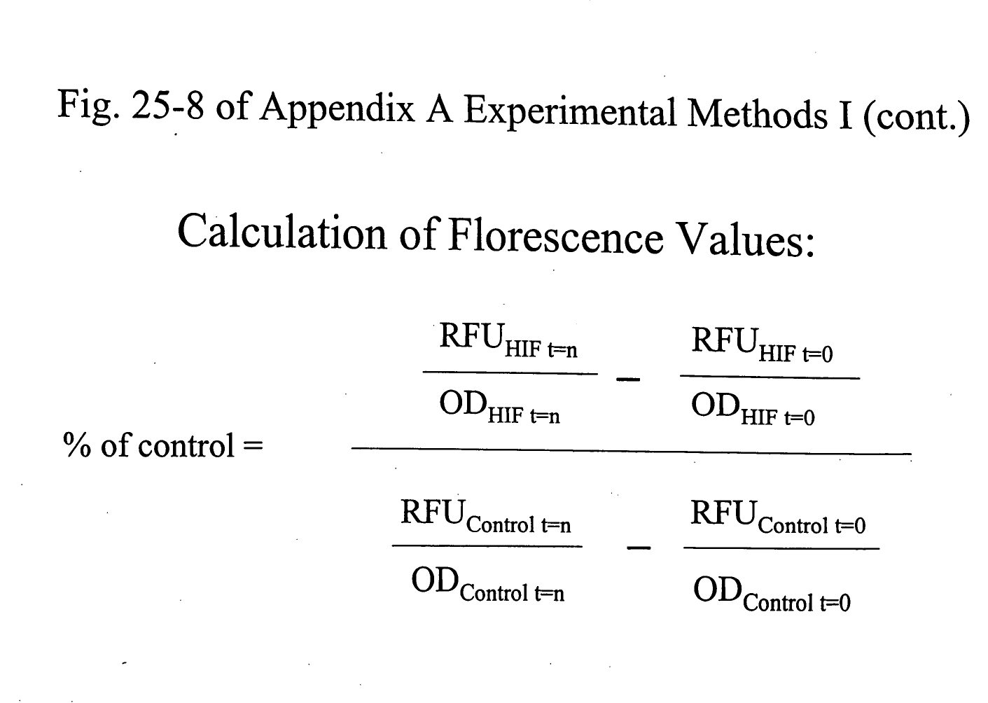

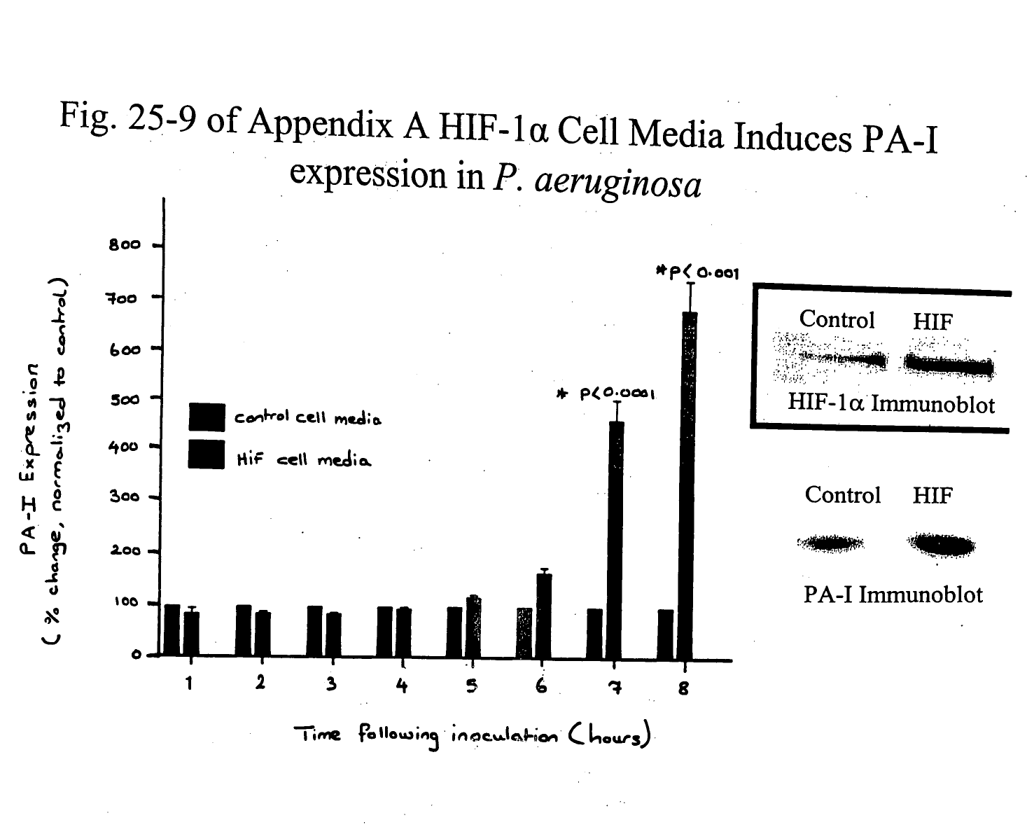

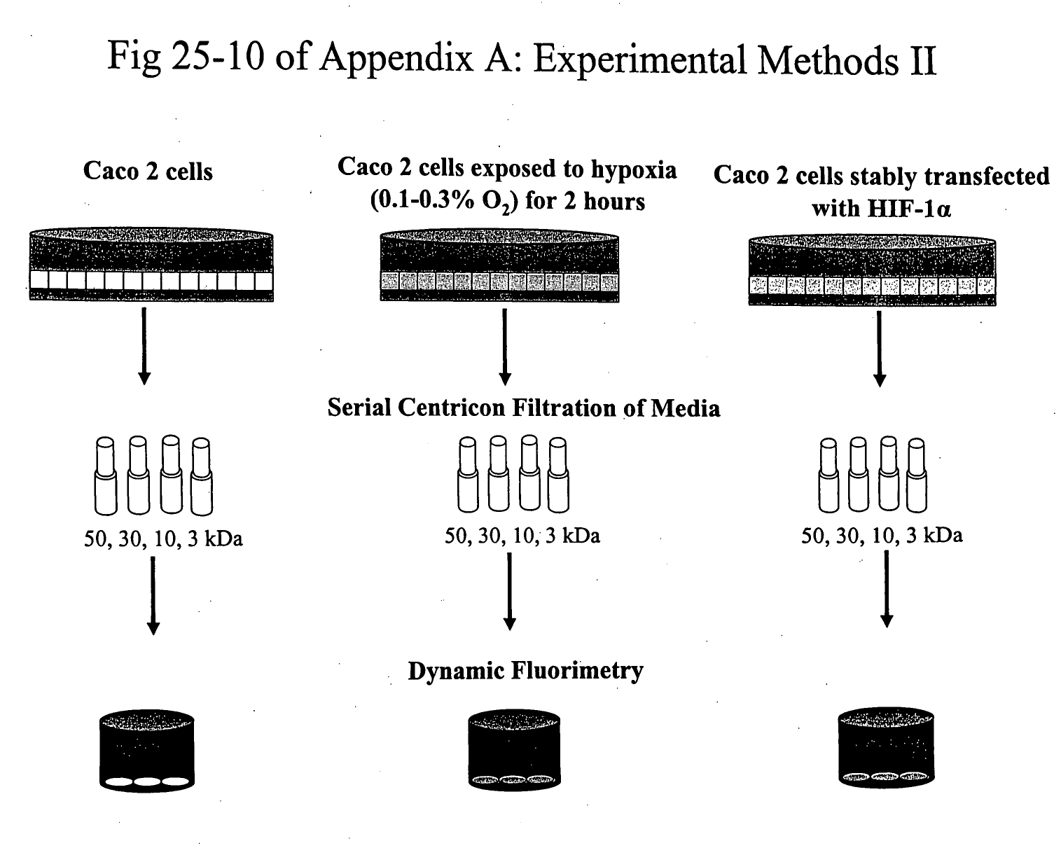

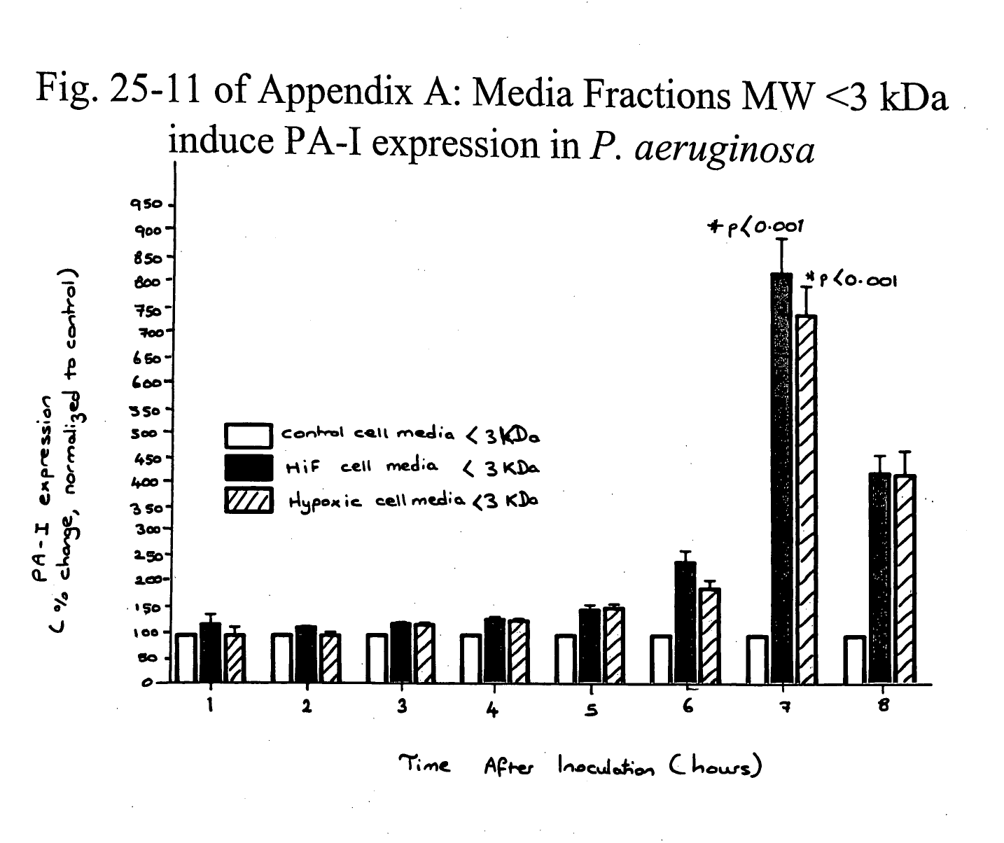

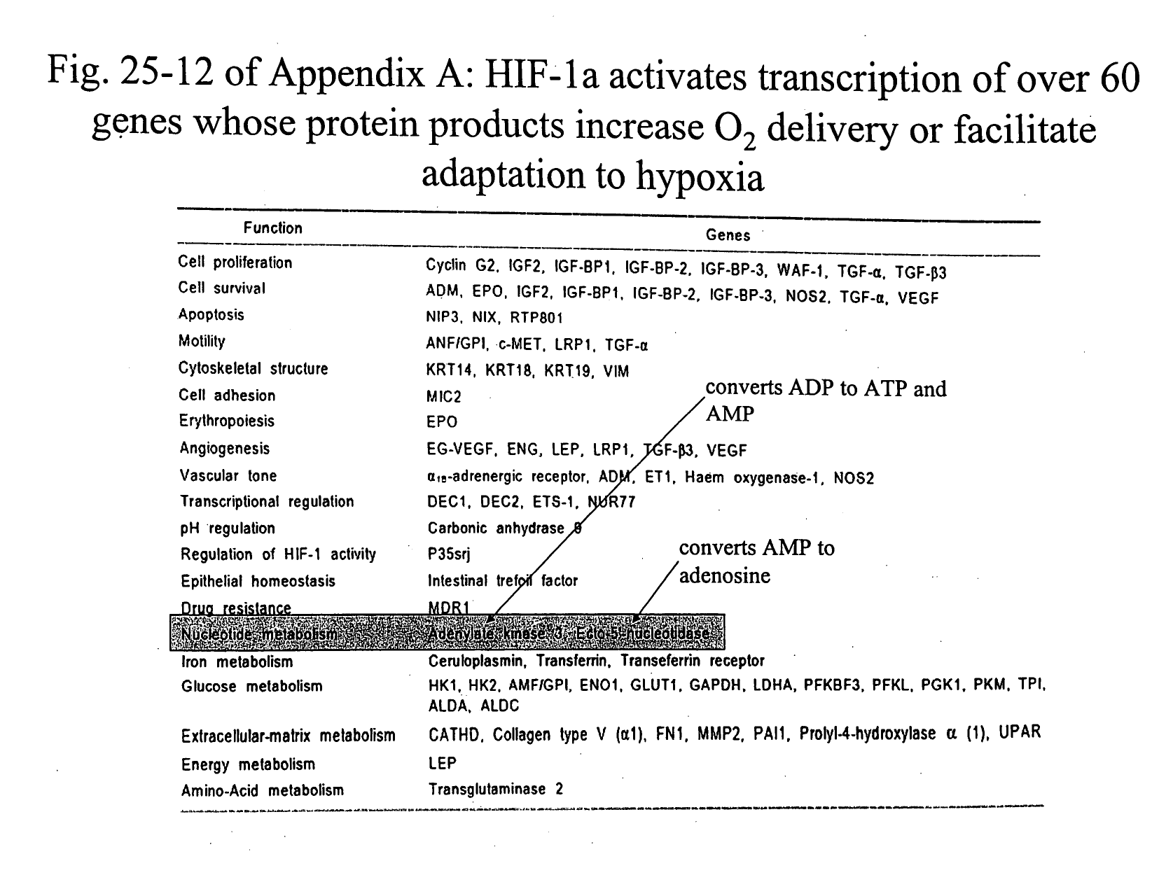

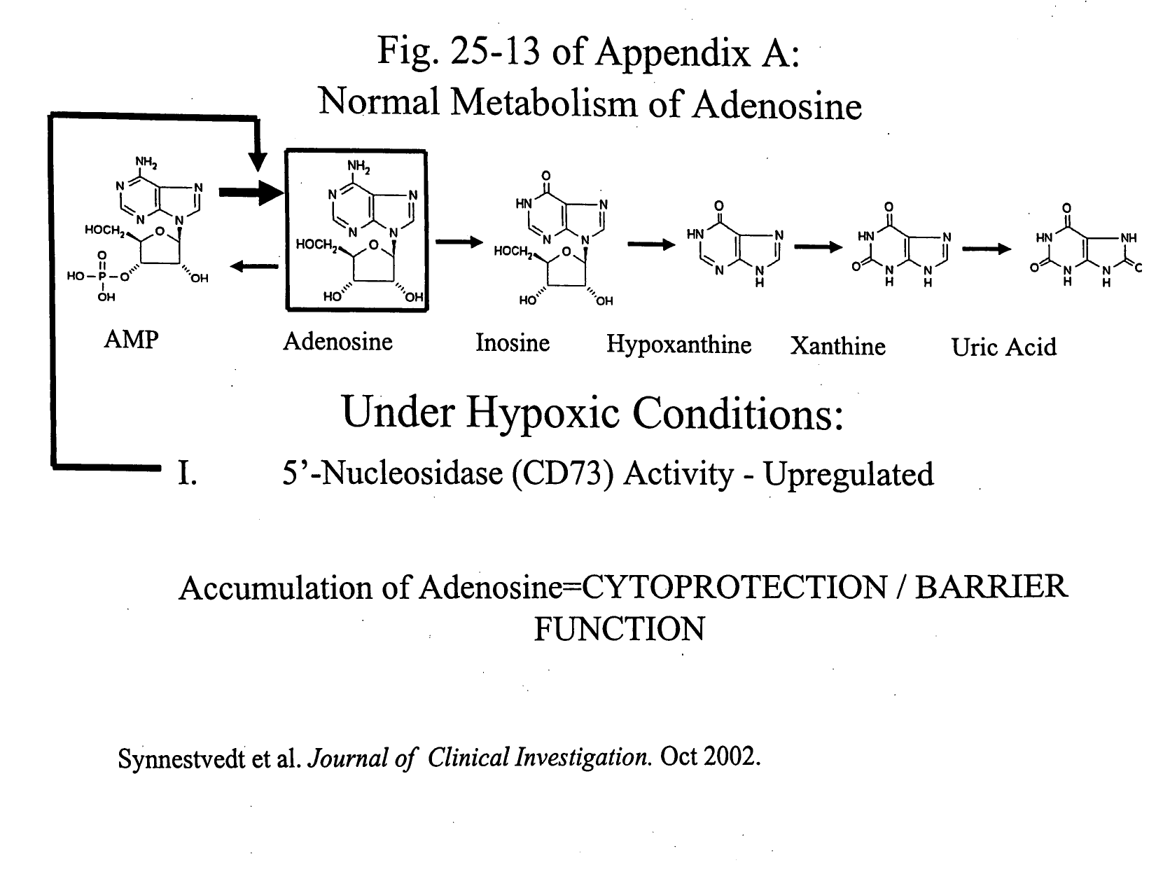

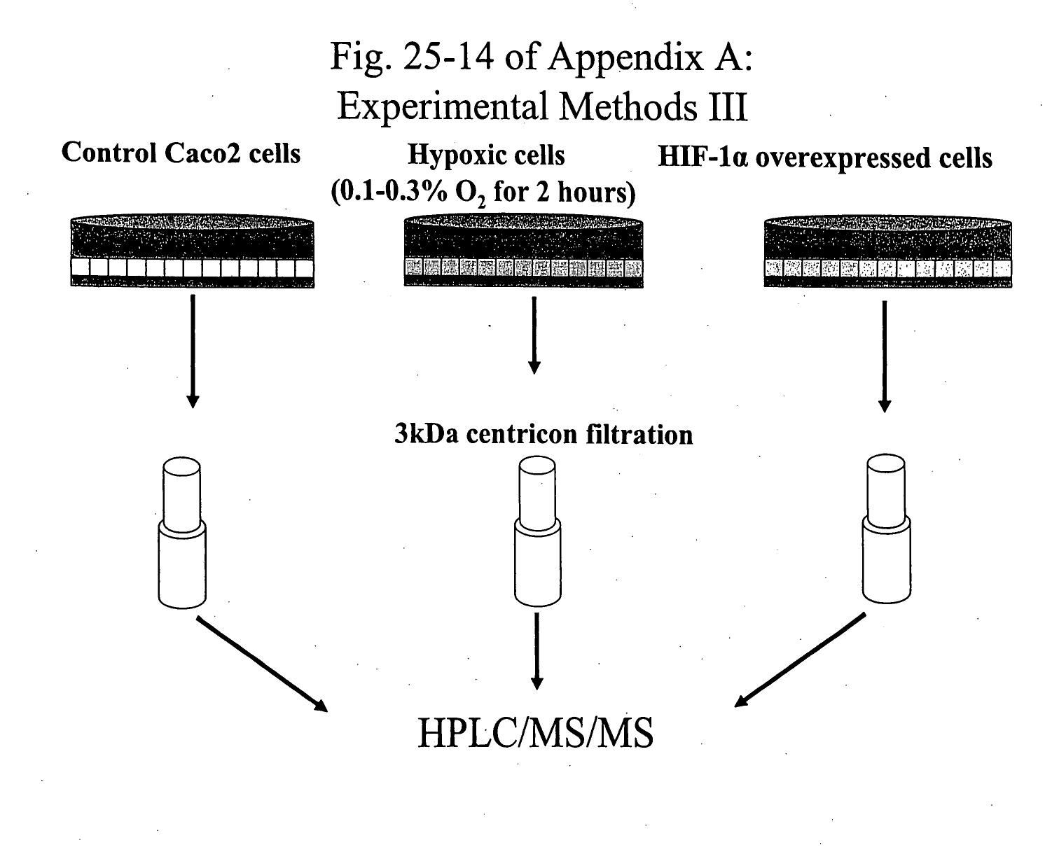

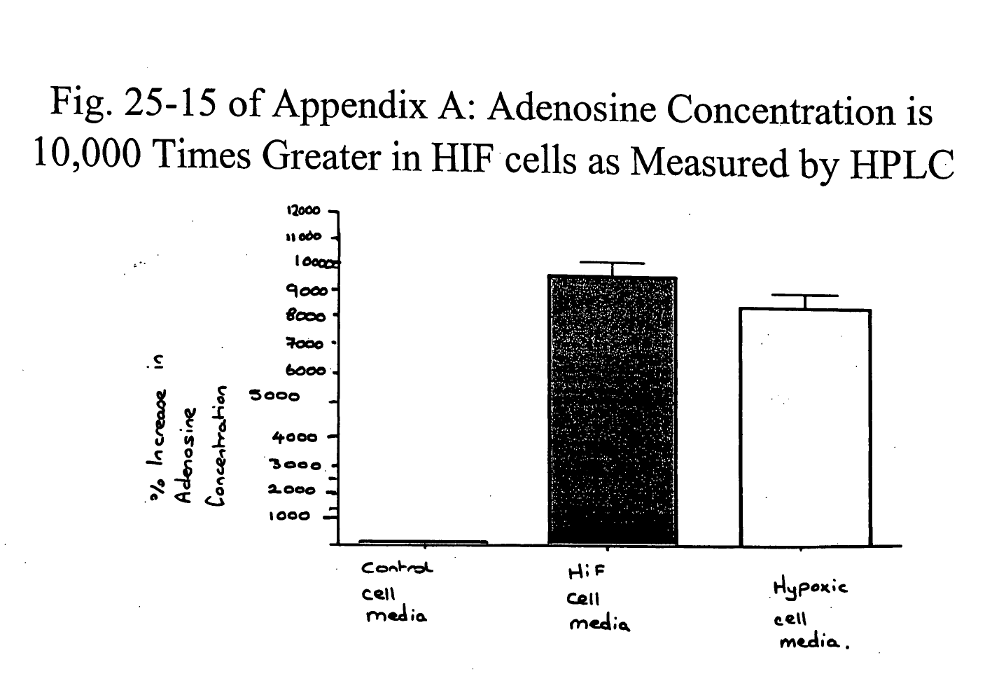

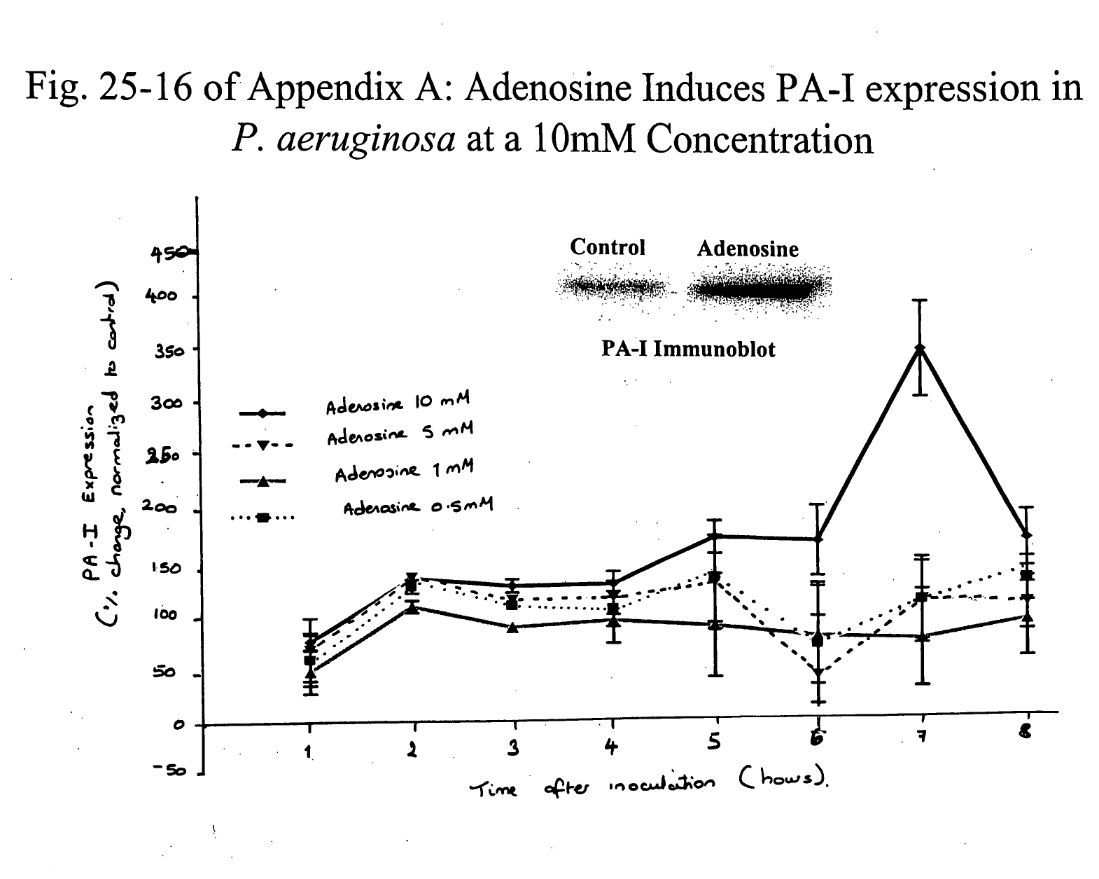

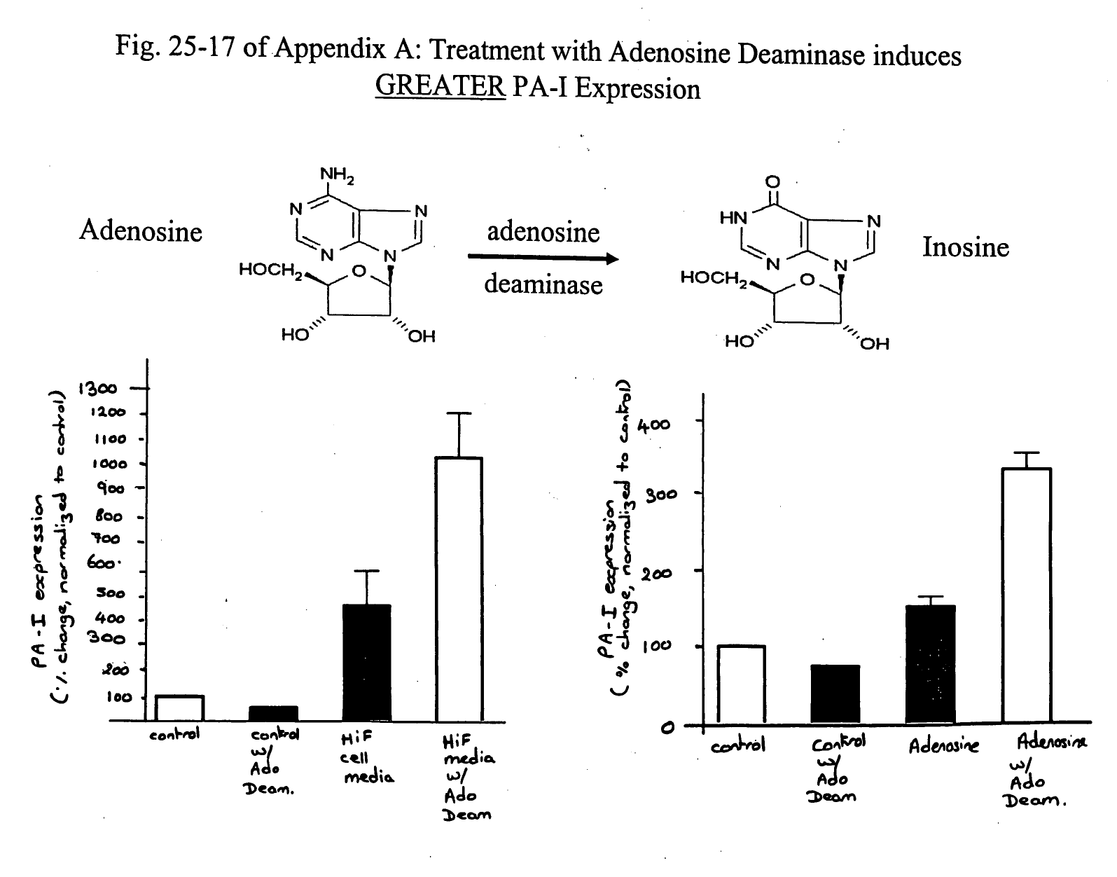

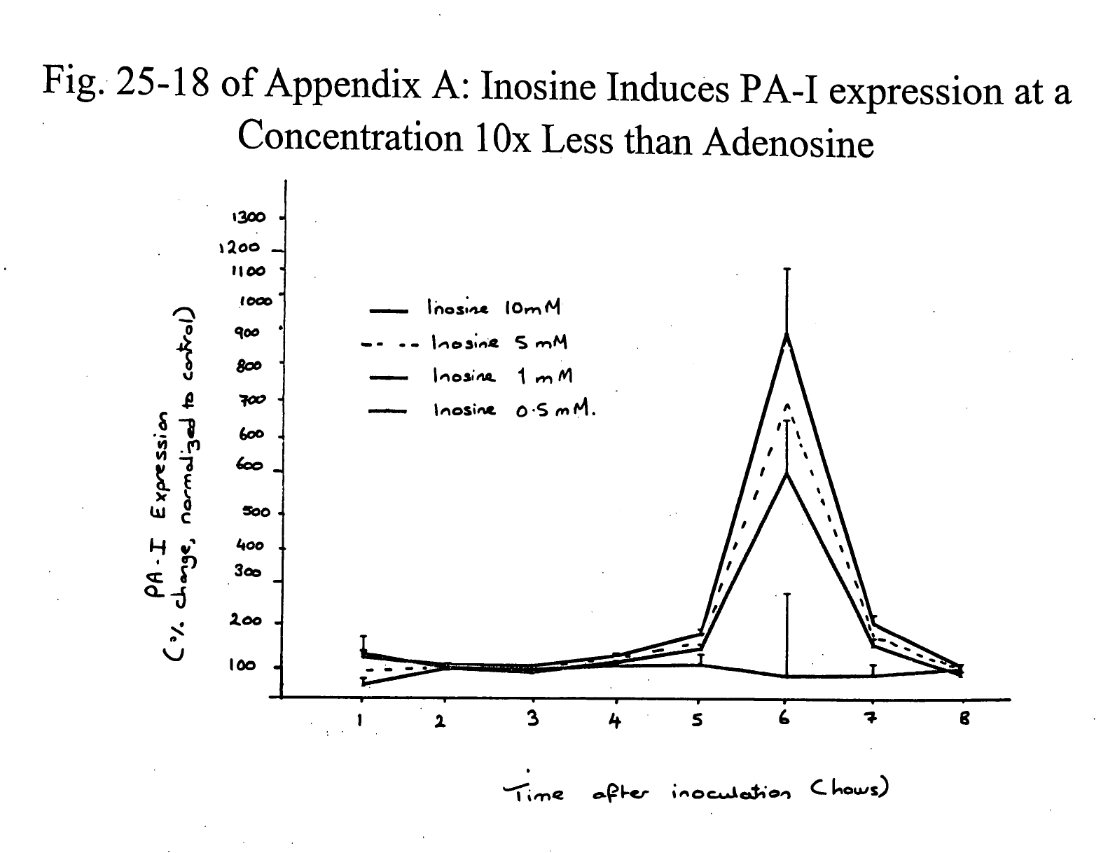

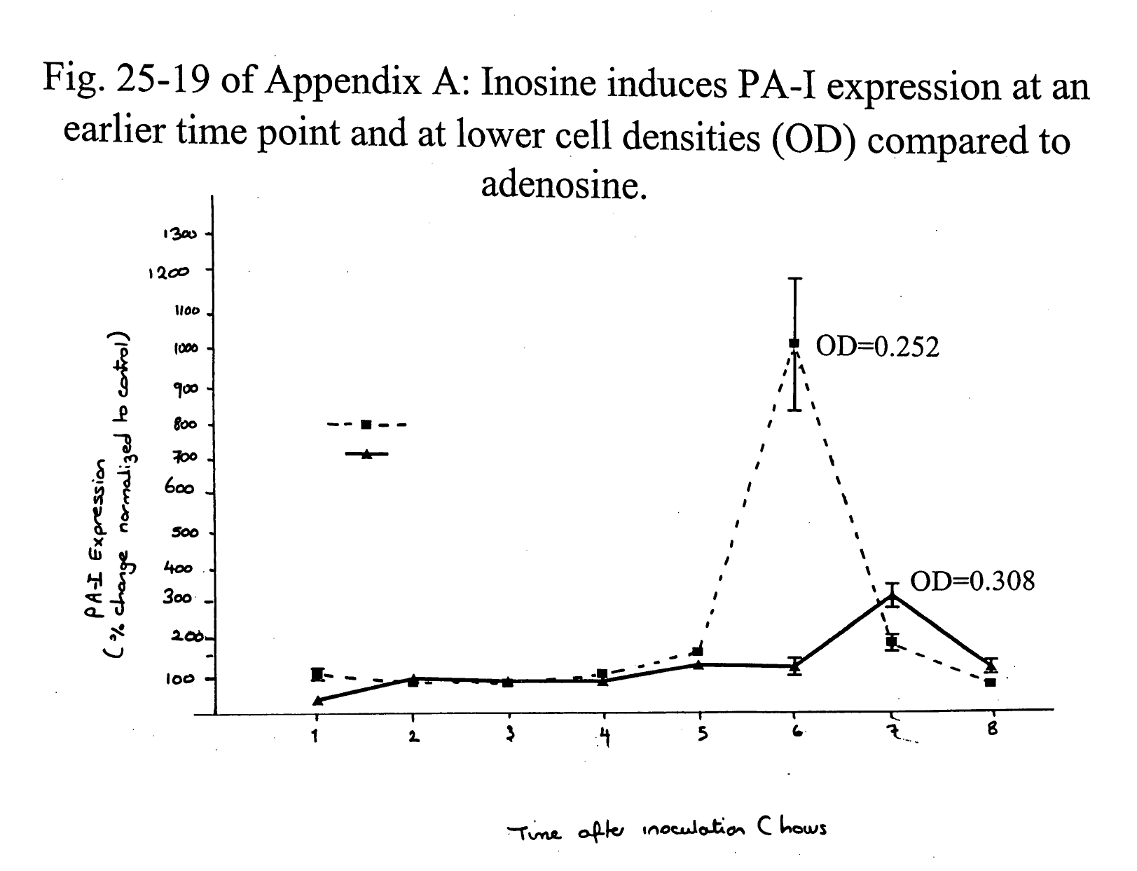

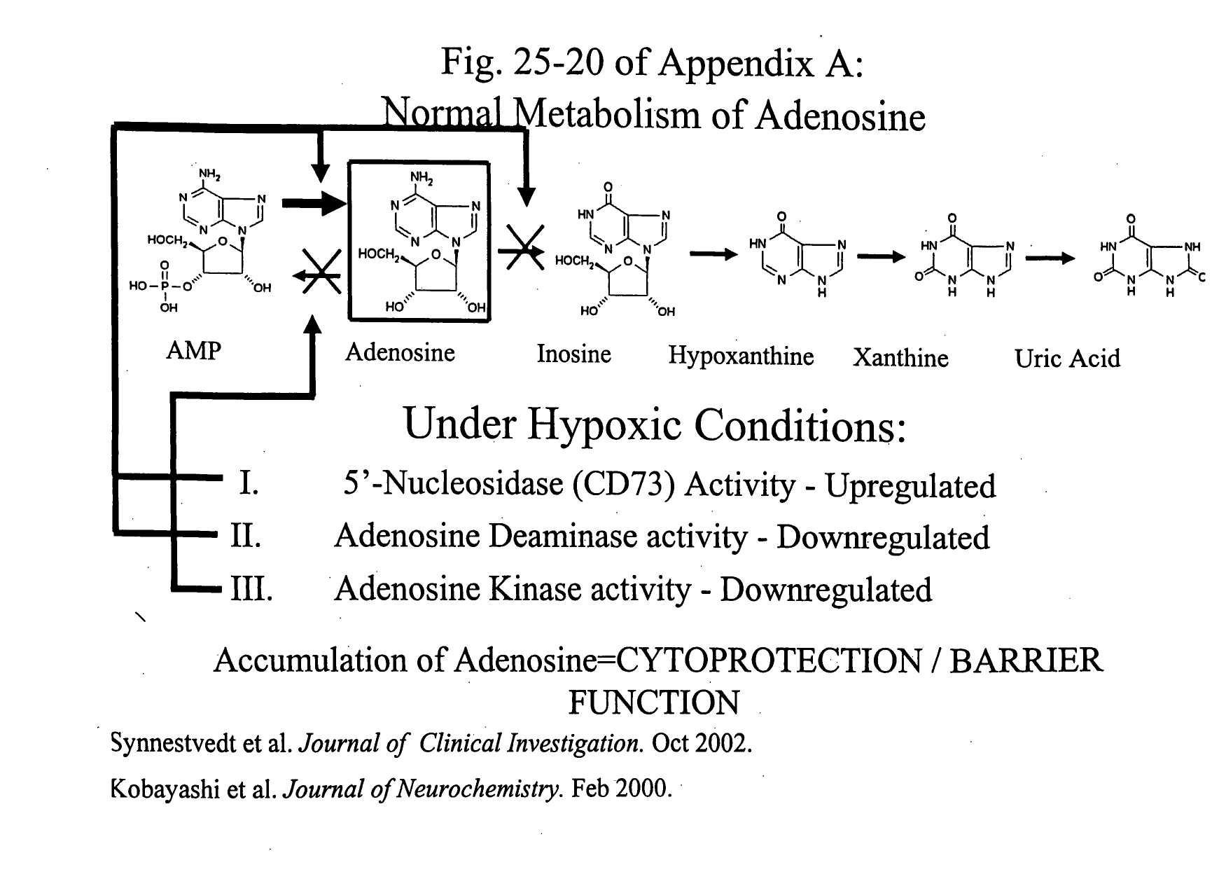

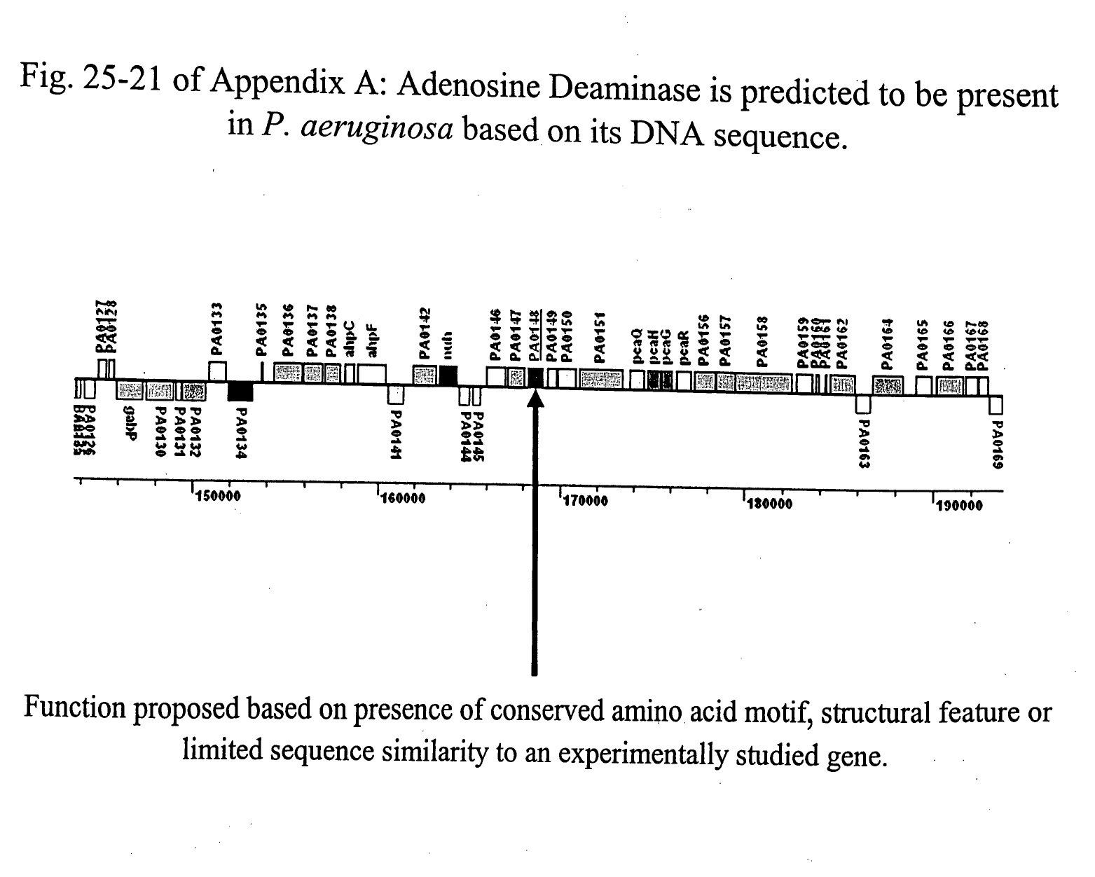

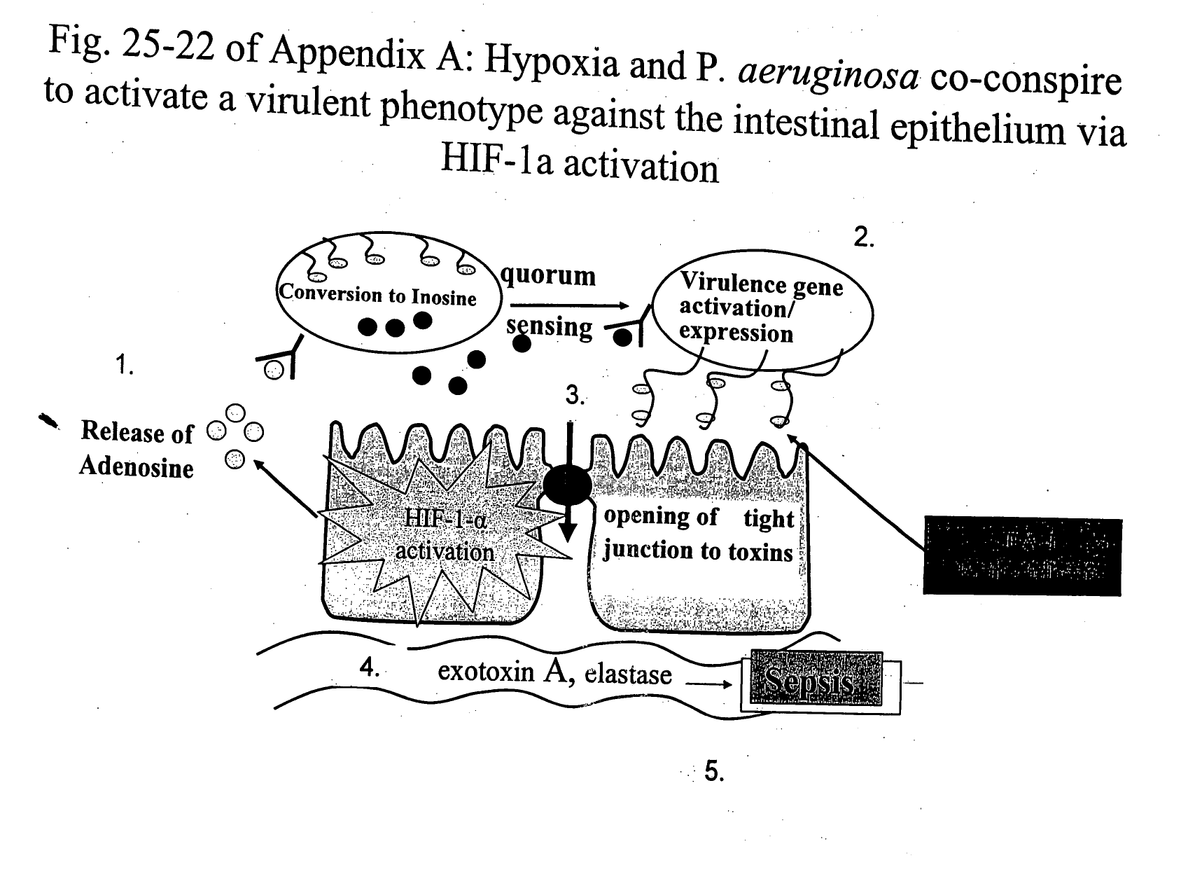

IFN-γ direct binding to a protein on the surface of P. aeruginosa, in the course of virulence activation, was also investigated. ELISA binding assays were performed by first coating microtiter plates with P. aeruginosa (strain PAO1), then adding recombinant human IFN-γ (rH IFN-γ), followed by biotin-labeled anti-IFN-γ antibody. IFN-γ avidly bound to whole fixed cells of P. aeruginosa in a dose-dependent manner (FIG. 25A). The ELISA data were confirmed by the results of immunofluorescent imaging of bacterial cells exposed to IFN-γ followed by biotin-labeled anti-IFN-γ antibody and Alexa 594-labeled streptavidin. The vast majority of bacterial cells (73%±3.2% vs. 8.5%±2.5%) bound IFN-γ (FIG. 25B, FIG. 27). The binding capacity of the IFN-γ to the P. aeruginosa was affected by bacterial growth phase (FIG. 28A). In order to localize the binding site of IFN-γ to P. aeruginosa (PAO1), equal protein concentrations of membrane and cytosol fractions of P. aeruginosa were prepared and coated onto ELISA microtiter plates. ELISA binding assays showed that IFN-γ preferentially bound to membrane fractions of P. aeruginosa (FIG. 28B). To determine if the observed membrane binding by IFN-γ was protein dependent, membrane fractions were treated with proteinase K for 3 hours and IFN-γ binding assessed. Binding by IFN-γ to P. aeruginosa membranes after treatment with proteinase K was decreased (FIG. 28C) suggested that IFN-γ binds to protein on the bacterial cell membrane. We next determined if other cytokines similarly would bind to P. aeruginosa cell membranes by performing reiterative binding studies with human TNF-α, IL-2, IL-4, IL-10, EGF, and TGF-β. No binding was observed with any of these cytokines (FIG. 28D). Taken together these data indicate IFN-γ bound to membrane protein on P. aeruginosa.

To isolate the putative protein to which IFN-γ binds on the cell membrane of P. aeruginosa, membrane proteins solubilized with mild detergents were initially shown to retain their binding capacity to IFN-γ by ELISA (FIG. 25C). Prior to isolation of the putative binding protein of IFN-γ, we sought to determine whether IFN-γ bound to single or multiple membrane proteins. Membrane proteins were then separated by non-denaturing gel electrophoresis, transferred to PVDF membranes and hybridized with IFN-γ followed by biotin-labeled anti-IFN-γ antibody. Results demonstrated a single immunoreactive band of about 35 kD. immunoreactivity was IFN-γ dose-dependent (FIG. 25D). In order to identify the putative binding protein, membrane protein was extracted from 4 L of freshly grown P. aeruginosa and fractionated by molecular weight between 10-100 kD. Solubilized protein was then immunoprecipitated using IFN-γ and anti-IFN-γ antibody. BSA was used as a control. Immunoprecipitation resulted in the appearance of a distinct protein with a molecular weight of about 35 kD. To further confirm that the protein isolated by immunoprecipitation was dependent on the presence of IFN-γ, equally divided solubilized membrane protein fractions were mixed with and without IFN-γ and then immunoprecipitated with anti-IFN-γ antibody. The 35 kD band appeared only in the solubilized membrane protein mixed with IFN-γ (FIG. 25E). The IFN-γ-dependent band was identified by ESI-TRAP-Electrospray LC-MSMS Ion Trap as the P. aeruginosa outer membrane porin OprF (FIG. 25F). These data established that IFN-γ binds to the P. aeruginosa outer membrane protein OprF (A. O. Azghani, S. Idell, M. Bains, R. E. Hancock, Microb Pathog 33, 109 (September 2002)).

To verify that OprF represented the major binding site for IFN-γ in P. aeruginosa strain PAO1, solubilized membrane proteins from OprF knockout strains of P. aeruginosa strain PAO1 (M. A. Jacobs et aL, Proc Natl Acad Sci USA 100, 14339 (Nov. 25, 2003)) were tested for their ability to bind IFN-γ in comparison to the wild-type strain using the established ELISA and immunoprecipitation technique. ELISA binding assays of solubilized membrane proteins demonstrated reduced binding of IFN-γ in OprF⁻ strains (FIG. 26A). Immunoprecipitation of solubilized membrane protein using IFN-γ and specific antibody confirmed the role of OprF by showing complete loss of the approximately 35 kD band in the OprF mutant strain (FIG. 26B). To verify the functional role of OprF on the responsiveness of P. aeruginosa to IFN-γ, we examined the expression of the PA-I protein in wild-type and OprF mutant strains exposed to 100 ng/ml of IFN-γ. Results demonstrate that mutant strains failed to increase the expression of the PA-I protein in response to an effective stimulating dose of IFN-γ as compared to the wild-type strain (FIG. 26C). The results from reporter gene fusion of wild-type and OprF mutant strains also demonstrated that IFN-γ activated PA-I expression through OprF (FIG. 26D). To further verify the role of OprF, OprF was reconstituted in mutant P. aeruginosa strain 31899 using the plasmid pUCP24/OprF. Reconstituted strains demonstrated recovery of their responsiveness to IFN-γ with an increase in PA-I protein expression (FIG. 26E). Finally, we verified the binding between OprF and IFN-γ by showing that purified OprF directly binds human IFN-γ (FIG. 26F) in a dose-dependent manner.

### Example 12

Identification of Host Stress-Derived BSCs by screening candidate Agents: the Role of Endogenous Opioids

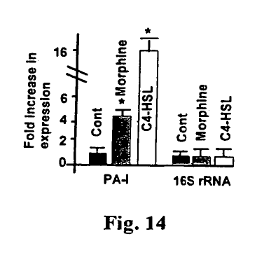

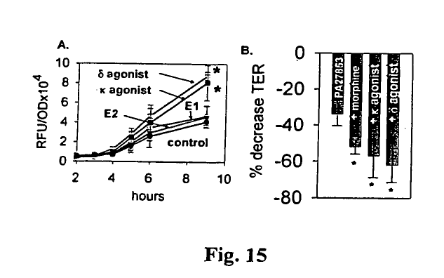

Although it was known that the counter-regulatory hormone, norepinephrine, increased the binding of P. aeruginosa to human O erythrocytes, there has been no information relating to the involvement of PA-I in the process. Accordingly, an assay to detect the presence of extracellular PA-I¹⁵ was performed. It was possible that norepinephrine would function as a host BSC for P. aeruginosa and, thus, affect human O erthyrocytes in a manner similar to the way it affected E. coli⁴⁸. Despite extensive analyses, PA-I expression was not affected by this compound. The screening of other catecholamines, all without positive results, led to the expectation that opioids, particularly morphine alkaloids, would activate PA-I. Endogenous morphine has been documented to be released in direct proportion to the magnitude of surgical stress/injury in both animals and humans⁵⁰. Initially, morphine was assessed for its effects. Interestingly, exposure of Pseudomonas strain PA27853 to physiologic concentrations of morphine (13 μM) resulted in a four-fold increase in PA-I expression (FIG. 14). As morphine is considered to be a non-selective opioid⁶⁴, specific endogenous opioid agonists with high selective affinity to μ, κ and δ receptors were tested for their abilities to induce PA-I lectin/adhesin expression in strains PA27853 and PAO1. Also tested were two pure μ peptide agonists, endomorphine-1 (E1) (Tyr-Pro-Trp-Phe-NH₂) and endomorphine-2 (E2) (Tyr-Tyr-Pro-Phe-Phe-NH₂), the potent K opioid non-peptide agonist U-50488, and the potent δ opioid non-peptide agonist BW373U86 for their respective abilities to induce PA-I expression in the reporter strain P. aeruginosa PA27853/PLL-EGFP (FIG. 15A). Results demonstrated that agonists targeting the κ and δ receptors had the greatest effect on PA-I expression as judged by increased fluorescence of the GFP reporter strain. In order to determine if PA-I was functionally expressed when exposed to the various opioid agonists, the agonists were tested for their abilities to shift the barrier dysregulating dynamics of PA27853 in MDCK cells. Results show that all three of the opioids that induced PA-I expression (morphine, κ and δ agonists), shifted the virulence of PA27853 as judged by a more profound decrease in the TEER of MDCK cells following apical exposure (FIG. 15B).

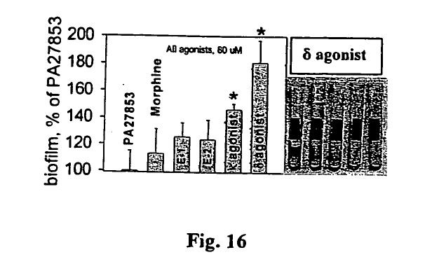

In order to determine if morphine could shift the in vivo virulence of P. aeruginosa, mice were implanted with slow release morphine pellets that release a daily dose of morphine that is similar to that used clinically (pellets obtained from the National Institute on Drug Abuse (NIDA)⁶⁵. Control mice were implanted with a placebo pellet. Mice drank infant formula spiked with a daily inoculum of 1×10⁸ cfu/ml of PA27853. All the morphine treated mice developed severe sepsis (4/4) and significant mortality while none of the control mice appeared septic and all survived. Finally, agonists were tested for their ability to induce biofilm in PA27853, a quorum sensing dependent phenotype⁶⁶. Biofilm production by P. aeruginosa and other organisms has been established to be a major phenotype indicative of enhanced virulence⁶⁷. The opioid agonists κ and δ agonists significantly increased biofilm production in strains PA27853 (FIG. 16). Taken together, these studies demonstrate that opioid agonists can directly influence the virulence, and potential lethality, of P. aeruginosa. It is expected that opioid agonists, whether found endogenously in a given cell type or not, and whether purified from a natural source, chemically synthesized, or produced by a combination thereof, are contemplated by the invention as useful modulators of the bacterial signaling affecting microbial pathogenesis generally, and eukaryotic (e.g., epithelial) cell barrier function more specifically.

### Example 13

Role of κ-Opioids in P. aeruginosa Virulence Expression

Opioid compounds, known to accumulate in tissues such as the lung and intestine following stress, directly activate the virulence of P. aeruginosa as judged by pyocyanin production, biofilm formation, and the expression of the PA-IL protein. Specifically, pyocyanin production was enhanced in the presence of the selective κ-opioid receptor agonist, U-50,488, and the naturally occurring endogenous peptide dynorphin, also a selective κ-opioid receptor agonist. To understand the regulatory pathway(s) involved in opioid-induced virulence gene expression in P. aeruginosa, the effect of U-50,488 on multiple mutant P. aeruginosa strains defective in key elements involved in pyocyanin production was examined. Results demonstrated that the global transcriptional regulator, MvfR, plays a key role in pyocyanin production in response to U-50,488. Intact MvfR was also shown to be required for P. aeruginosa to respond to C4-HSL, a key quorum sensing signaling molecule known to activate hundreds of virulence genes. Taken together, these studies indicate that opioid compounds serve as host-derived signaling molecules that can be perceived by bacteria during host stress for the purposes of activating their virulence phenotype.

Bacterial strains and culture conditions. P. aeruginosa strains PAO1 and 27853, and their derivative strains (Table 1) were routinely grown in tryptic soy broth (TSB) supplemented when necessary with tetracycline (Tc), 60 μg/ml, and/or gentamicin (Gm), 100 μg/ml. Alkaloid opiates morphine, a preferable μ-opioid receptor agonist (A. Shahbazian, et al., Br J Pharmacol 135, 741 (2002)), U-50,488, a specific κ-opioid receptor agonist (J. Szmuszkovicz, Prog Drug Res 53,1 (1999)), and BW373U86, a specific δ-opioid receptor agonist (S. F. Sezen, V. A. Kenigs and D. R. Kapusta, J Pharmacol Exp Ther 287, 238 (1998)), along with the peptide opioid dynorphin, a specific K-opioid receptor agonist (Y. Zhang, E. R. Butelman, S. D. Schlussman, A. Ho and M. J. Kreek, Psychopharmacology (Berl) 172, 422 (2004)), and specific K-opioid-receptor antagonist nor-binaltorphimine (A. Shahbazian, et al., Br J Pharmacol 135, 741 (2002)) were used in the experiments. Morphine was purchased from Abbott Laboratories, U-50,488, BW373U86, dynorphin, nor-binaltorphimine, and methyl anthranilate from Sigma-Aldrich, and C4-HSL from Fluka.

Complementation of MvfR mutant with mvfR gene. Amplified mvfr was directly cloned in pCR2.1 (Invitrogen), digested with XbaI-HindIII restriction endonucleases and subcloned into pUCP24 under the Plac promoter to create pUCP24/mvfR. The plasmids pUCP24 (blank control) and pUCP24/mvfR were electroporated in strain 13375, defective in MvfR production, to create the P. aeruginosa strain 13375/MvfR (Tables 1, 2).

Complementation of GacA mutant with gacA gene. The gacA gene, a member of a two-component signaling method involved in the elaboration of virulence in many gram-negative bacteria, was amplified and directly cloned into pCR2.1 (Invitrogen). The gene was then excised with XbaI-HindIII restriction endonucleases and subcloned into pUCP24 under the Plac promoter to create pUCP24/gacA. The plasmids pUCP24 (blank control) and pUCP24/gacA were electroporated in P. aeruginosa strain PA0628 1, defective in GacA production, to create the P. aeruginosa strain PA06281/GacA (Tables 1, 2).

Truncation of MvfR. PCR products of truncated mvfR genes amplified from pUCP24/MvfR and their respective primers (Tables 1, 2) were purified using a Geneclean kit (Qbiogene), digested with XbaI-HindIII restriction endonucleases, and ligated into pUCP24 followed by electroporation into P. aeruginosa strain 13375.

Pyocyanin assay. Bacteria were grown in TSB at 37° C. under shaking conditions at 220 rpm, with opioid compounds added at the early exponential phase of bacterial growth (OD_{600 nm} of about 0.15-0.2). After incubation, pyocyanin was extracted from culture media in 6 chloroform followed by re-extraction in 0.2 M HCl, and measured at OD520 nm as described (D. W. Essar, L. Eberly, A. Hadero and I. P. Crawford, J Bacteriol 172, 884 (1990)).

PA-IL assays. immunoblotting and fluorescence of the GFP-PA-IL reporter strain were used to determine the effect of opioids on PA-IL expression. For immunoblotting, P. aeruginosa PAO1 was grown in TSB media with or without 100 μM U-50,488, and cells were collected at the late exponential phase of growth (OD600 nm=1.8). Equal amounts of protein from each sample were separated by 15% SDS-PAGE, transferred to a PDF membrane, and probed with affinity-purified rabbit polyclonal anti-PA-IL antibodies. The probed membranes were treated with anti-rabbit horsardish peroxidase-conjugated IgG, and developed using SuperSignal West Femto chemoluminescent substrate (Pierce). For PA-IL expression detected by fluorescence, a bacterial culture of the GFP-PA-IL reporter strain 27853/PLL-EGFP (L. Wu, et al., Gastroenterology 126, 488 (2004)) was plated at a final concentration of 108 CFU/ml in a 96-well fluorometry plate (Costar) in HDMEM media containing 10% FBS and HEPES buffer with or without 60 μM of U-50,488. Incubation was performed at 37° C., 100 rpm, and fluorescence reading was performed hourly with a 96-well fluorometry Plate Reader (Synergy HT, Biotec Inc.) at excitation/emission of 485/528 nm. Fluorescence intensity was normalized to cell density measured at 600 nm.

Biofilm formation assay. Bacterial cells were plated in quadruplicate in 96-well Ubottom plates (Falcon) at a concentration of 107 CFU/ml in M63S media (13.6 g KH2PO41-1, 2.0 g (NH4)2SO41-1, 0.5 mg FeSO4×7H2O 1-1), supplemented with 0.5% casamino acids, 1 mM MgSO4×7H2O and 0.2% glucose, and incubated overnight at 37° C. under static conditions. U-50,488 was added at the inoculation point. After inoculation, the wells were rinsed thoroughly with water and the attached material was stained with 0.1% crystal violet, washed with water, and solubilized in ethanol. Solubilized fractions were collected and absorbance measured at 590 nm as described (G. A. O'Toole and R. Kolter, Mol Microbiol 28, 449 (1998)) with a Plate Reader.

κ-Opioid Receptor Agonists U-50,488 and Dynorphin Stimulate Pyocyanin Production in P. aeruginosa

P. aeruginosa harvested from the intestine of surgically stress mice appeared intensely green compared to P. aeruginosa from the intestines of sham-operated control mice. Thus, P. aeruginosa might be responding to a signal to produce increased amounts of pyocyanin (PCN) in response to environmental cues unique to the intestinal tract of stressed mice. Pyocyanin, a redox active compound that increases intracellular oxidant stress, has been shown to play a key role in the virulence of P. aeruginosa in animal models mediating tissue damage and necrosis during lung infection (G. W. Lau, H. Ran, F. Kong, D. J. Hassett and D. Mavrodi, Infect Immun 72, 4275 (2004)). P. aeruginosa PAO1 was exposed to peptide opioids and alkaloid opiates representing groups of μ-, κ-, and δ-opioid receptor agonists. Results indicated that following overnight exposure, the alkaloid opiate U-50,488, a specific K-opioid receptor agonist, induced an intensely bright green color in P. aeruginosa PAOI, while no such effect was observed with any of the remaining compounds (FIG. 29A). To verify that the color change was due to PCN production, pyocyanin was measured at OD520 nm (D. W. Essar, L. Eberly, A. Hadero and I. P. Crawford, J Bacteriol 172, 884 (1990)). Results demonstrated that U-50,488 induced a dose-dependent effect on PCN production that was observed with P. aeruginosa strains PAO1 and 27853 (FIG. 29B). Exposure of P. aeruginosa to dynorphin, a naturally occurring specific κ-opioid receptor peptide agonist, also enhanced PCN production in a dose-dependent manner (FIG. 29C). Reiterative experiments performed in the presence of the specific κ-opioid receptor antagonist norbinaltorphimine (NOR), demonstrated that NOR attenuates enhanced PCN production in PAO1 following exposure to U-50,488 in a dose-dependent manner and completely inhibits enhanced PCN production at a dose of 200 μM (FIG. 29D).

The κ-Opioid-Receptor Agonist U-50,488 Shifts Pyocyanin Production at Lower Cell Densities in P. aeruginosa.

We assessed the dynamics of PCN production in response to U-50,488 at varying cells densities, since the expression of QS-dependent genes is known to occur at high bacterial cell densities when QS signaling molecules reach their threshold concentrations. As a positive control, bacteria were exposed to C4-homoserine lactone (C4-HSL), a QS signaling molecule involved in PCN regulation (M. R. Parsek and E. P. Greenberg, Proc Natl Acad Sci U S A 97, 8789 (2000)). We found that exposure of PAO1 to U-50,488 had a similar effect to exposure of cells to C4-HSL, resulting in a shift in the production of PCN at lower cell densities (FIG. 30). Neither compound had an effect on bacterial growth in TSB media.

The κ-Opioid-Receptor Agonist U-50,488 Exerts its Inducing Effect on Pyocyanin Production Via Elements of the Quorum Sensing System in Pseudomonas aeruginosa.

A summary of the pathways of PCN regulation and biosynthesis (D. V. Mavrodi, et al., J Bacteriol 183, 6454 (2001), E. Deziel, et al., Proc Natl Acad Sci U S A 101, 1339 (2004), T. R. de Kievit, Y. Kakai, J. K. Register, E. C. Pesci and B. H. Iglewski, FEMS Microbiol Lett 212, 101 (2002), S. L. McKnight, B. H. Iglewski and E. C. Pesci, J Bacteriol 182, 2702 (2000)) is depicted in FIG. 3 1A. In order to define potential pathways by which U-50,488 induces PCN production, mutant strains defective in key genes involved in PCN production were exposed to U-50,488 and the effect on pyocyanin production was measured. First, mutants defective in genes encoding core elements of the QS system (J. P. Pearson, E. C. Pesci and B. H. Iglewski, J Bacteriol 179, 5756 (1997)) (lasR, lasI, rhlI, rhIR) were analyzed and the results demonstrated that exposure to U-50,488 did not restore PCN production (relative to non-mutant strains) in any of these mutants (FIG. 31B). The roles of the global virulence regulators GacA and MvfR on PCN production were then investigated. Both GacA (C. Reimmann, et al., Mol Microbiol 24, 309 (1997)) and MvfR (E. Deziel, et al., Proc Natl Acad Sci U S A 101, 1339 (2004)) have been shown to play a major role in PCN production in P. aeruginosa. Neither AGacA nor AMvfR produced PCN, as expected, and exposure to U-50,488 could not restore PCN production (FIG. 31C). C4-HSL was also unable to restore PCN production in the gacA and mvfR mutants (FIG. 31D). The finding that C4-HSL did not restore PCN production in the GacA mutant is consistent with the finding that the analogous QS molecule, N-hexanoyl-HSL (C6-HSL), did not restore phenazine production in a ΔGacA mutant of P. aureofaciens (S. T. Chancey, D. W. Wood and L. S. Pierson, 3rd, Appl Environ Microbiol 65, 2294 (1999)). Seven additional mvfR mutants from the comprehensive transposon library (M. A. Jacobs, et al., Proc Natl Acad Sci U S A 100, 14339 (2003)) (i.e., numbers 8902, 47418, 35448, 51955, 21170, 47853, and 47198) were exposed to C4-HSL in order to confirm this finding. Results demonstrated that none of these mutants produced PCN in the presence of 1 mM C4-HSL.

MvfR is Involved in the Ability of U-50,488 and C4-HSL to Enhance PCN Production in PAO1.

In order to define the possible role of MvfR and GacA in the U-50,488-mediated upregulation of PCN synthesis, we complemented ΔMvfR and ΔGacA with their respective genes on the multicopy plasmid pUCP24 (S. E. West, H. P. Schweizer, C. Dall, A. K. Sample and L. J. Runyen-Janecky, Gene 148, 81 (1994)). Both complemented mutants produced significantly higher amounts of PCN (FIGS. 32A,B). The addition of C4-HSL and U-50,488 further increased the already elevated PCN production in ΔMvfR /mvfR (FIG. 32C). In contrast, PCN production in ΔGacA/gacA was decreased, albeit minimally, when exposed overnight to either 1 mM U-50,488 or 100 μM C4-HSL (FIG. 32D). Dynamic tracking of PCN production in the complemented mutant ΔMvfR/mvfR exposed to U-50,488 and C4-HSL demonstrated a shift in PCN production at lower cell densities (FIG. 32E), similar to that observed in the parental strain PAO1 (FIG. 30). The gacA complemented mutant, ΔGacA/gacA, itself produced PCN at lower cell densities than those observed with the parental strain PAO1. Exposure of ΔGacA/gacA to C4-HSL had no effect on the dynamics of PCN production whereas exposure to U-50,488 delayed PCN production. (FIG. 32F). These results indicate that MvfR isinvolved in the up-regulation of PCN production by exogenously applied U-50,488 and C4-HSL.

Intact Substrate-Binding and DNA-Binding domains of MvfR are Required for U-50,488 to Enhance PCN Production in PAO1.

MvfR belongs to a family of prokaryotic LysR transcriptional regulators that possess a helix-turn-helix DNA-binding motif at the N terminus and a substrate binding domain at the C terminus. A NCBI Conserved Domain Search revealed similar domains in MvfR: a LysR DNA-binding domain located at 6-64 aa, and a LysR substrate binding domain located at 156-293 aa (FIG. 33A). Therefore we constructed PAOI mutants producing N- and C-terminus-truncated MvfR to determine if specific domains could be identified that play a functional role in mediating the K-opioid receptor agonist effect on PCN production. Results indicate that the mutant lacking amino acids 121-332, defective in the DNA-binding domain, did not produce any PCN, and did not respond to U-50,488 or C4-HSL (FIG. 33B). Mutants lacking either amino acids 1-299 or 1-293, truncated at their C termini without affecting the substrate binding domain, produced PCN and responded to U-50,488 and C4-HSL with enhanced PCN production. Further deletions, however, including amino acids Arg293, Leu292, and Phe284, did affect the substrate binding domain in mutants 1-292, 1-291, and 1-283. All three mutants failed to produce PCN and did not respond to U-50,488 and C4-HSL (FIG. 33B). These results confirm a key functional role for MvfR in mediating enhanced PCN production in P. aeruginosa in response to U-50,488 and C4-HSL.

The Effect of U-50,488 on PCN Production is Dependent on MvfR-Regulated Synthesis of Pseudomonas Quinolone Signal (PQS).

MvfR might play a critical role in PCN production via positive transcriptional regulation of the phnAB and PQS ABCDE operons that encode two 12 precursors of PQS, anthranilic acid (AA) and 4-hydroxy-2-heptylquinolone (HHQ) (E. Deziel, et al., Proc Natl Acad Sci U S A 101, 1339 (2004)). Therefore the mutants ΔPhnA and ΔPqsA were examined for their ability to produce PCN in the presence of U-50,488. Neither mutant produced PCN. Exposure of each mutant to U-50,488 resulted in a slight increase in PCN production, although the increase was much less than that observed with the wild-type strain PAO1 (FIG. 34A). These data suggested that MvfR-regulated PQS synthesis may be important for the ability of U-50,488 to enhance PCN production. Finally, reiterative experiments were performed with a P. aeruginosa mutant defective in the phzA1 gene, which is part of the operon that contains the core genes for PCN biosynthesis and that is directly preceded by the lux box (D. V. Mavrodi, et al., J Bacteriol 183, 6454 (2001)). ΔPhzA1 produced no PCN even when exposed to U-50,488 (FIG. 34A).

To confirm that PQS plays a role in the pathway by which U-50,488 enhances PCN production, U-50,488 was applied to P. aeruginosa incubated with 2 mM methyl anthranilate (MA), a compound previously shown to inhibit PQS synthesis in P. aeruginosa (S. P. Diggle, et al., Mol Microbiol 50,29 (2003), M. W. Calfee, J. P. Coleman and E. C. Pesci, Proc Natl Acad Sci U S A 98, 11633 (2001)). Results demonstrated that MA inhibited the ability of U-50,488 to enhance PCN production in PAO1 (FIG. 34B). These findings indicate that U-50,488 triggers PCN production in P. aeruginosa via a signal transduction pathway that includes the activation of transcriptional regulator MvfR and the synthesis of the MvfR-regulated molecule, PQS.

U-50,488 Stimulates Other QS-Regulated Virulence Determinants in P. aeruginosa Including Biofilm Formation and PA-IL Production.

To determine if other QS-dependent phenotypes could be expressed in response to U-50,488, we measured biofilm production (T. R. De Kievit, R. Gillis, S. Marx, C. Brown and B. H. Iglewski, Appl Environ Microbiol 67, 1865 (2001)) and PA-IL lectin expression (K. Winzer, et al., J Bacteriol 182, 6401 (2000), M. Schuster, M. L. Urbanowski and E. P. Greenberg, Proc Natl Acad Sci U S A 101, 15833 (2004)) in P. aeruginosa exposed to this opiate. U-50,488 enhanced biofilm formation in PAO1 in a concentration-dependent manner (FIG. 35A). PA-IL expression was dynamically tracked in response to U-50,488 using the green fluorescent PA-IL reporter strain P. aeruginosa 27853/PLL-EGFP (L. Wu, et al., Gastroenterology 126, 488 (2004)). Marked fluorescence was observed in this strain following 9 hours of growth in HDMEM media (FIG. 35B). Results were confirmed in strain PAO1 by immunoblotting using rabbit polyclonal antibody against PA-IL (FIG. 35C).

The Effect of U-50,488 on PCN Production in P. aeruginosa can be Inhibited by the Anti-Infective High Molecular Weight Polymer PEG 15-20.

A high molecular weight polymer, PEG 15-20, protects mice against lethal sepsis due to P. aeruginosa by interfering with the ability of both host elements (epithelial cell contact) and the QS signaling molecule C4-HSL to enhance P. aeruginosa virulence without affecting bacterial growth (L. Wu, et al., Gastroenterology 126, 488 (2004)). The capacity of PEG 15-20 to interfere with the U-50, 488 effect on P. aeruginosa was assessed by measuring PCN production in the media of P. aeruginosa PAOI incubated in the presence of 5% PEG 15-20 and 0.5 mM U-50,488 or 0.2 mM C4-HSL. Results demonstrated that PEG 15-20 had a strong inhibitory effect on both U-50, 488- and C4-HSL-mediated up-regulation of PCN production.

TABLE 1
Bacterial strains

| P. aeruginosa strains | Relevant genotype | Source |
|---|---|---|
| PA27853 | Wild type | Alverdy's library |
| PAO1 | Wild type | Dr. Iglewski |
| PAO-JP-1 | ΔLasI (lasl::Tc^{r}) | Dr. Iglewski (21) |
| PAO-R1 | ΔLasR (lasR::Tc^{r}) | Dr. Iglewski |
| PDO100 | ΔRhlI (rhlI::Tn501) | Dr. Iglewski |
| PAO-MW1 | ΔRhlIΔLasI (rhlI::Tn501 lasI::tetA) | Dr. Greenberg (30) |
| PAO44488 | ΔRhlR (rhlR::ISphoA/hah) | PAO1 transposon library (24) |
| PAO6281 | ΔGacA (gacA::Sp^{r}/Sm^{r}) | Dr. Reimmann (22) |
| PAO6281/pUCP24/GacA | ΔGacA complemented with gacA | This study |
| | on pUCP24 | |
| PAO6281/pUCP24 | ΔGacA transformed with blank | This study |
| | pUCP24 | |
| PAO8902 | ΔMvfR (mvfR::ISlacZ/hah) | PAO1 transposon library |
| PAO47418 | ΔMvfR (mvfR::ISphoA/hah) | PAO1 transposon library |
| PAO35448 | ΔMvfR (mvfR::ISphoA/hah) | PAO1 transposon library |
| PAO51955 | ΔMvfR (mvfR::ISphoA/hah) | PAO1 transposon library |
| PAO21170 | ΔMvfR (mvfR::ISlacZ/hah) | PAO1 transposon library |
| PAO47853 | ΔMvfR (mvfR::ISphoA/hah) | PAO1 transposon library |
| PAO47198 | ΔMvfR (mvfR::ISphoA/hah) | PAO1 transposon library |
| PAO13375 | ΔMvfR (mvfR::ISlacZ/hah) | PAO1 transposon library |
| PAO13375/pUCP24/MvfR | ΔMvfR complemented with mvfR | This study |
| | on pUCP24 | |
| PAO13375/pUCP24 | ΔMvfR transformed with blank | This study |
| | pUCP24 | |
| PAO53589 | ΔPqsA (pqsA::ISphoA/hah) | PAO1 transposon library |
| PAO37309 | ΔPhzA (phzA::ISphoA/hah) | PAO1 transposon library |
| PAO47305 | ΔPhzA1 (phzA1::ISphoA/hah) | PAO1 transposon library |
| PAO3375/pUCP24/MvfR 1-299 | ΔMvfR complemented with pUCP24 | This study |
| | harboring mvfR truncated with 33 aa | |
| | at C terminus | |
| PAO13375/pUCP24/MvfR | ΔMvfR complemented with pUCP24 | This study |
| 1-293 | harboring mvfR truncated with 39 aa | |
| | at C terminus | |
| PAO13375/pUCP24/MvfR | ΔMvfR complemented with pUCP24 | This study |
| 1-292 | harboring mvfR truncated with 40 aa | |
| | at C terminus | |
| PAO13375/pUCP24/MvfR | ΔMvfR complemented with pUCP24 | This study |
| 1-291 | harboring mvfR truncated with 41 aa | |
| | at C terminus | |
| PAO13375/pUCP24/MvfR | ΔMvfR complemented with pUCP24 | This study |
| 1-283 | harboring mvfR truncated with 49 aa | |
| | at C terminus | |
| PAO13375/pUCP24/MvfR | ΔMvfR complemented with pUCP24 | This study |
| 121-332 | harboring mvfR truncated with 120 aa | |
| | at N terminus | |
| 27853/PLL-EGFP | Green fluorescent PA-IL reporter | Alverdy (13) |
| | strain | |

TABLE 2

Primers designed for complementation
and truncation

| | | |
|---|---|---|
| Strain | Template | Primers |

| | | |
|---|---|---|
| 13375/ | PAO1 DNA | forward |
| MvfR | | 5′-AAGGAATAAGGGATGGCTATTCA-3′ |
| | | reversed |
| | | 5′-CTACTCTGGTGGGGCGCGCTGGC-3′ |
| PAO281/ | PAO1 DNA | forward |
| GacA | | 5′-CGACGAGGTGCAGCGTGATTAAGGT-3′ |
| | | reversed |
| | | 5′-CTAGCTGGCGGCATCGACCATGC-3′ |
| 13375/ | pUCP24/ | MvfrXbaI |
| 1-299 | mvfR | 5′-GCTCTAGAAAGGAATAAGGGATGCCTAT-3′ |
| | | C33HindIII |
| | | 5′-CCCAAGCTTCTAACGCTGGCGGCCGAGT |
| | | TC 3′ |
| 13375/ | pUCP24/ | MvfrXbaI |
| 1-293 | mvfR | 5′-GCTCTAGAAAGGAATAAGGGATGCCTAT-3′ |
| | | C39HindIII |
| | | 5′-CCCAAGCTTCTAGCGCAGGCGCTGGCGG |
| | | GC-3′ |
| 13375/ | pUCP24/ | MvfrXbaI |
| 1-292 | mvfR | 5′-GCTCTAGAAAGGAATAAGGGATGCCTAT-3′ |
| | | C40HindIII |
| | | 5′-CCCAAGCTTCTACAGGCGCTGGCGGGCG |
| | | CT-3′ |
| 13375/ | pUCP24/ | MvfrXbaI |
| 1-291 | mvfR | 5′-GCTCTAGAAAGGAATAAGGGATGCCTAT-3′ |
| | | C41HindIII |
| | | 5′-CCCAAGCTTCTAGCGCTGGCGGGCGCTT |
| | | TC-3′ |
| 13375/ | pUCP24/ | N120XbaI |
| 121-232 | mvfR | 5′-GCTCTAGAAAGGAATAAGGGATGGTCAGCC |
| | | TGATACGC-3′ |
| | | MvfRHindIII |
| | | 5′-CCCAAGCTTCTACTCTGGTGCGGCGCGCTG |
| | | GC-3′] |

### Example 13

P. aeruginosa PAO1 Expresses Abundant PA-I and Alters MDCK Monolayer Permeability in a PA-I Dependent Manner

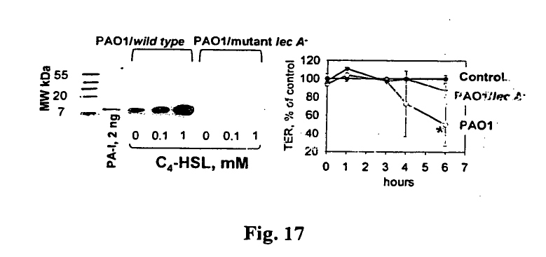

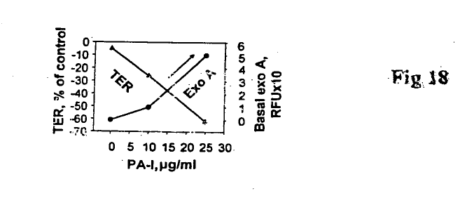

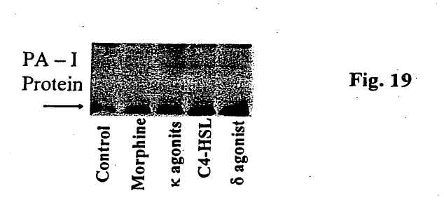

In order to verify that the sequenced P. aeruginosa strain, PAO1, expressed PA-I, and to verify that strains altered the TEER of MDCK cells in a PA-I-dependent manner, both wild type and PA-I mutant strains deleted of the PA-I gene (leca) (obtained from the comprehensive transposon mutant library⁶⁸), were assayed for PA-I protein expression and their abilities to decrease MDCK monolayer TEER. PA-I protein expression is highly abundant and responds to varying doses of C4-HSL, its cognate quorum sensing signaling molecule (FIG. 17). In addition, in this strain, the ability of P. aeruginosa to decrease MDCK monolayer integrity (TEER) is highly dependent on the expression of PA-I (FIG. 17). Also, it was determined that the PA-I induced permeability defect in MDCK cells was of sufficient magnitude to permit the apical to basolateral flux of exotoxin A across the monolayers (FIG. 18). Finally PA-I protein has been shown to be abundantly expressed in PAO1 when strains were exposed to the various opioid agonists (FIG. 19). For PA-I protein, the 6 agonist induced a response equal to C4-HSL (FIG. 19). The data establish that PA-I expression affects eukaryotic cell barrier function. Thus, it is expected that modulators of PA-I expression, as well as modulators of PA-I activity, will be useful in affecting the virulence phenotype of microbial pathogens and will be useful in affecting the eukaryotic (e.g., epithelial) cell barrier dysfunction associated with that phenotype. Moreover, such modulators will be useful in treating a variety of mammals, and particularly humans, exhibiting disorders or diseases characterized by permeability defects, including epithelial cell disorders exhibiting an epithelial cell barrier dysfunction; the modulators will also be useful in ameliorating at least one symptom of such a disorder and in preventing such a disorder.

### Example 14

Host Cell-Derived Bacterial Signaling Components Enhance the Barrier Dysregulating Properties of P. aeruginosa Against Epithelial Cells

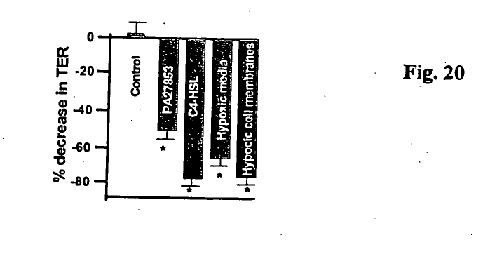

In order to demonstrate that host stress BSCs could shift the barrier dysregulating dynamics of P. aeruginosa against the epithelium, media and cell membrane fractions from Caco-2 cells exposed to hypoxia were added to the apical wells of MDCK cells apically inoculated with PA27853. TEER was measured over time. C4-HSL was also added to serve as a positive control for PA-I expression. Both media and cell membranes enhanced the barrier dysregulating properties of P. aeruginosa (PA27853) against MDCK cells at four hours (FIG. 20). None of the host cell derived bacterial signaling compounds alone had any effect on MDCK TEER. The results demonstrate that the microbial pathogen (e.g., P. aeruginosa) is necessary to alter the barrier function of host cells.

### Example 15

PA-I is Expressed In Vivo within the Digestive Tube of Caenorhabditis elegans

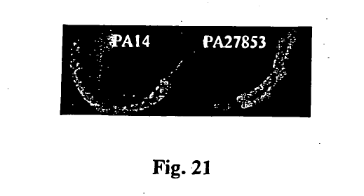

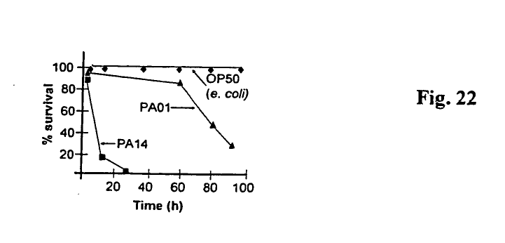

The PA-I-GFP reporter plasmid was introduced into P. aeruginosa strain PA14, a strain highly lethal to C. elegans⁵⁶, by electroporation. Worms were then fed GFP-tagged PA14 and PA27853 and examined for fluorescent bacteria. Worms feeding on lawns of PA14 and PA27853 displayed fluorescent bacteria within the digestive tube, whereas no fluorescence was seen within the surrounding media, indicating that PA-I promoter activity is activated by local factors within the worm digestive tube(FIG. 21). Finally the killing dynamics of strain PA-14, a highly lethal strain in this model⁵⁶, was compared to the dynamics associated with the completely sequenced PAO1 strain. As seen in FIG. 22, the strain of E. coli (OP50) upon which worms normally feed, resulted in 100% survival, whereas, PA-14 displayed fast killing dynamics, as predicted. The PAO1 strain displayed slow killing with only a 50% mortality rate at 80 hours. Thus PAO1 exhibits killing dynamics that will allow assessments of whether host stress-derived BSCs shift the killing curve to that of a more virulent strain. It is expected that BSCs, whether soluble or membrane-bound, will shift the killing dynamics of relatively quiescent, or benign, microbes towards the dynamics exhibited by lethal microbial strains. Stated in the alternative, it is expected that a BSC will shift the phenotype of a microbe towards a virulent phenotype. Modulators of such activities are expected to be useful in preventing and treating disorders associated with the display of a virulence phenotype by any such microbe and in particular by P. aeruginosa. Such modulators are also expected to be used in methods for ameliorating a symptom of such a disorder.

### Example 16

P. aeruginosa Genes Involved in BSC-Induced PA-I Lectin/Adhesin Gene Expression

The data demonstrate that i) morphine, the potent opioid agonists U-50488 and BW373U86, which target κ and δ receptors, respectively, and IFN-γ, induce a robust response in P. aeruginosa strains PA27853 and PAO1 to express PA-I; ii) PA-I expression is dependent on multiple elements of the virulence gene regulatory circuitry in P. aeruginosa, including the quorum sensing signaling system (QS) and RpoS. The data will show the genes that are required for opioids and IFN-γ to elicit a PA-I response in P. aeruginosa and will facilitate a determination of whether these host stress-derived BSCs use common genes and membrane receptor proteins to activate PA-I expression.

A. Genes Required for P. aeruginosa PA-I Expression Responsive to Morphine, κ and δ Opioid Agonists, and IFN-γ

At least two techniques are contemplated for use in gene identification: 1) perform transcriptome analysis on P. aeruginosa strain PAO1 exposed to morphine, κ and δ opioid receptor agonists, and IFN-γ, and 2) establish a functional role for candidate genes identified in the transcriptome analysis by screening the corresponding transposon mutants for their ability to up-regulate PA-I protein expression in response to opioids and IFN-γ.

Transcriptome Analysis

Genes in strain PAO1 whose expression is increased in the presence of opioids and/or IFN-γ will constitute the initial focus. Transcriptome analyses is performed using Affymetrix GeneChip genome arrays in strain PAO1 to identify the genes that respond to the host cell elements such as morphine (non-selective opioid receptor agonist), U-50488 (κ receptor agonist) , BW373U86 (δ opioid receptor agonist), and IFN-γ. Time and dose variables for the following experiments are based on data for PA-I expression (mRNA) in strain PAO1.

Briefly, bacteria are grown in TSB overnight and diluted 1:100 in TSB containing either morphine (20 μM), K agonist (80 μM), δ agonist (80 μM), or IFN-γ (10 μg/ml). Bacteria are then grown to an OD₆₀₀ of 0.5, 1.0, and 2.0, representing three stages of growth: exponential phase, late exponential phase, and stationary phase, respectively. These three time points will permit the capture of genes that are expressed early in the PA-I signaling pathway as well as during time points of high cell density. For transcriptome analysis, RNA is isolated from bacterial cells (treated and non-treated with morphine, κ and δ opioid receptor agonists, and IFN-γ) at the three designated points in the growth phase. cDNA synthesis, fragmentation, labeling, and hybridization, as well as P. aeruginosa GeneChip genome array processing, are performed as described herein or as known in the art. Each experiment is preferably performed in triplicate.

Functional Analysis of Candidate Genes

Genes showing at least a 2.5-fold change in expression resulting from exposure to morphine, κ and δ opioid receptor agonists, and/or IFN-γ, are individually tested for their specific role in PA-I protein expression by screening mutant strains from a PAO1 transposon library⁶⁸ (University of Washington Genome Center, see below) using dot blot analysis, as described⁷⁴. Briefly, strains are grown in sequential runs using 384-well microtiter plates at 2 separate bacterial cell densities (OD₆₀₀ of 1.0 and 2.0) predetermined to respond to the inducing compound (opioids, IFN-γ). Dose-response curves are generated with varying doses of the PA-I inducing compounds at different bacterial cell densities in wild-type strains and in several mutant strains to determine the optimal conditions for screening. Experiments are performed separately for morphine, U-50488, BW373U86, and IFN-γ. Briefly, either morphine, U-50488, BW373U86, or IFN-γ are added to the wells containing mutant strains at the predetermined dose. All runs are performed with the wild-type strain as a control. The PA-I-inducing compound is added to the well for a predetermined time. Next, the supernatant is removed and the bacterial cell pellet is lysed by the addition of lysis solution directly into the well. The entire 384-well plate is then spun down (4000 g) and the supernatant transferred to an Immobilon P-PDF membrane using a 384 replicator. Membranes are then treated with anti-PA-I primary and secondary antibodies. Dot blot analysis allows for rapid identification of all of the mutant strains that do not up-regulate PA-I in the presence of host stress-derived bacterial signaling compounds, thereby identifying genes that are required for PA-I expression. All assays are preferably performed in triplicate (3 cell densities×5 groups (4 experimental+1 control)×triplicate (3) assays=45 gene arrays).

It is expected that many of the genes that have already been established to play a role in PA-I expression, including genes in the QS and RpoS regulon, will be identified. However, it is expected that new and unanticipated functions for known genes will also be identified. Further, if CyaB or GacS transcripts are increased in response to opioids or IFN-γ, and if Cya B and GacS transposon knockouts do not respond to either opioids or IFN-γ with an increase in PA-I, then the role of these established biosensors as two-component regulators of opioids or IFN-γ signaling to P. aeruginosa will be confirmed. Combining the results of the transcriptome analyses with the functional analyses of the transposon library will allow us to determine whether opioids and IFN-activate common membrane biosensors and common downstream genes involved in PA-I expression. It is possible that one or more of the non-peptide opioids diffuses directly into the bacterial cell cytoplasm where it initiates gene activation downstream of the two-component membrane biosensors. If this is the case, then all of the transposon knockout strains encoding membrane proteins are expected to respond with an increase in PA-I and microarray data will demonstrate that levels of transcripts encoding membrane proteins will be unaltered by either opioids or IFN-γ. However, it is possible that membrane biosensors are constitutively expressed and therefore gene expression will not change in response to opioids or IFN-γ. If this is the case, then the entire transposon library will be screened for PA-I expression in response to opioids or IFN-γ, approaches that are feasible given the high-throughput nature of the Dot-Blot technique⁷⁴⁻⁷⁶. Of note here is that gene expressions can be triggered at different times during culturing and can respond to an exogenous compound(s) to varying degrees depending on the concentration of compound. The genomically sequenced strain PAOI makes abundant PA-I and the anti-PA-I lectin/adhesin antibodies are highly specific.

The data demonstrate that opioid receptor agonists and IFN-γ signal P. aeruginosa to express PA-I mRNA and protein. In addition, these PA-I signaling compounds induce P. aeruginosa to express a more virulent phenotype against the epithelium. The genes that control PA-I expression are dependent on two key global regulatory systems that activate hundreds of virulence genes in P. aeruginosa. The activation of these interconnected systems of virulence gene regulation are directly influenced by membrane biosensors that recognize elements of host cells and include, but are not limited to, CyaB and GacS, via a hierarchical cascade involving the transcriptional regulators Vfr and Gac A. Genes that are differentially expressed in response to opioids and IFN-γ will be identified using an unbiased transcriptome analysis approach. This approach was chosen instead of pursuing individual candidate genes involved in known pathways of PA-I expression because all previous studies have been performed only at high cell densities and in the absence of any host cell elements. Accordingly, previously described gene expression patterns may not be applicable in the physiologic models. The goal of this study is to identify and functionally validate the genes that are involved in PA-I expression in response to morphine, κ and δ opioid receptor agonists, and IFN-γ.

B. Identify the Receptors in P. aeruginosa that Bind Morphine and IFN-γ

The data show that a single solubilized membrane protein from P. aeruginosa can be isolated that avidly binds IFN-γ. In addition, morphine also binds to membrane protein fractions. Because antibody is available that specifically recognizes each of IFN-γ and morphine, initial studies are examining the effect of these two BSCs. Using the commercial antibodies, the membrane proteins that bind IFN-γ and/or morphine are identified, and optionally purified. This protein-based approach provides data which complements the experiments described above.

Two approaches available for use in identifying membrane proteins that bind IFN-γ and/or morphine are now described. First, membrane proteins of P. aeruginosa strain PAO1 are solubilized using mild detergents. The binding capacity of solubilized protein fractions for IFN-γ or morphine is then determined using simple ELISA binding assays. Protein fractions are then immunoprecipitated using the respective antibody and proteins are identified, e.g., by Maldi-MS.

Confirmation of the identity of a binding protein(s) is achieved by determining that a transposon knockout of the gene encoding the candidate protein(s) does not respond to IFN-γ or morphine with an increase in PA-I, using the techniques described herein. In order to confirm the function of candidate proteins showing fidelity in these two analyses, candidate proteins are re-expressed in the corresponding transposon knockout to verify that the PA-I response is re-established. Additionally, receptor antagonists may also be developed.

The data indicate that membrane receptors for morphine and IFN-γ can be identified by identifying proteins from solubilized membranes. A potential limitation using this technique is that morphine could diffuse directly into the bacterial cytoplasm and interact with a downstream target and not a membrane protein. This possibility is consistent with results demonstrating that morphine does not change the transcript profiles of any genes encoding membrane proteins, but the data for IFN-γ disclosed herein is inconsistent with this interpretation. In addition, morphine binding to a solubilized bacterial membrane protein was demonstrated using fluorescent imaging and analysis. Also, there is the possibility that transmembrane proteins or proteins that bind host stress-derived BSCs could be secreted into the culture medium and not be present within bacterial membranes. An example of such proteins are the bacterial iron binding proteins (enterochelin), which are released by bacteria into the culture medium and then re-enter the bacterial cells. Under such circumstances, the screening of cytosolic fractions and inner and outer membrane preparations are contemplated, along with iterative experiments probing for binding proteins with specific antibodies. Any discordance between the transposon mutant experiments and the proteins purified from bacterial membranes will be reconciled by analyzing IFN-γ-membrane protein or morphine-membrane protein interactions directly using surface plasmon resonance and mass spectrometry.

### Example 17

The Impact of Host Signaling on Microbial Virulence States

The data demonstrates that PA-I knockout strains (lecA⁻) do not decrease the TEER of cultured epithelial cells. The lethality of strains of P. aeruginosa exposed to opioid agonists and IFN-γ can be defined in vivo using the well-characterized invertebrate Caenorhabditis elegans and the established model of gut-derived sepsis in mice.

A. The Defect in Epithelial Barrier Function Induced by P. aeruginosa Exposed to Opioid Agonists and IFN-γ and the Role of PA-I in this Response

One issue is whether opioids or IFN-γ can activate P. aeruginosa to express a lethal phenotype against an epithelium, as judged by an increase in exotoxin A flux across epithelial cell monolayers, through the action of its PA-I lectin/adhesin.

To address that issue, MDCK cells are grown to confluence to maintain a stable TEER in transwells. Cells are apically inoculated with P. aeruginosa strain PAO1 (10⁷ cfu/ml) in the presence and absence of varying doses of morphine (about 20 μM), κ agonist (about 80 μM), κ agonist (about 80 μM), or IFN-γ (about 10 μg/ml). To optimize the effect of opioids and IFN-γ on the barrier dysregulating effect of P. aeruginosa against epithelial cells, dose and time response curves are generated. TEER is measured using chopstick electrodes hourly for 8 hours. The apical to basolateral flux of exotoxin A using Alexa-594 labeled exotoxin A is determined in iterative experiments performed at each hourly time point in order to correlate the decrease in TEER to exotoxin A flux for each condition. In selected experiments in which a significant permeability defect to exotoxin A is established, the specific role of PA-I is defined by performing iterative experiments in the presence and absence of 0.3% GalNAc (N-acetylgalactoside) and 0.6% mellibiose, two oligosaccharides that specifically bind to PA-I⁷⁸. Irrelevant sugars (heparin/mannose) are used as negative controls (see reference²⁴ (see Wu paper in Appendix). Iterative studies are also performed using the PA-I transposon knockout (lecA-) mutant to define the specific role of PA-I in strains exposed to opioids and IFN-γ. It is expected that PA-I will be expressed and localized to the microbial pathogen cell surface, where it will be situated in position to interact with host epithelial cells, thereby influencing, at a minimum, the cell barrier properties of the epithelial cells.

It is expected that opioids and IFN-γ will decrease the TEER of MDCK cells. Exotoxin A flux that is increased in cell monolayers with a low TEER will suggest that the opioids and IFN-γ alone can induce a lethal phenotype in P. aeruginosa. If the GalNAc, mellibiose inhibition studies, or the PA-I lectin/adhesin knockout strains, prevent P. aeruginosa from altering TEER and exotoxin A flux across the cell monolayers, then this will indicate that the observed response is PA-I-mediated. If the PA-I knockout mutant strains alter TEER and exotoxin A flux in response to opioids or IFN-γ, then this will indicate that PA-I alone may not be responsible for the virulence of P. aeruginosa against the intestinal epithelium. Data from these studies are directly compared and correlated to worm and mouse lethality studies (see below) to determine if these in vitro assays accurately predict a lethal phenotype in vivo, as expected.

### Example 18

The Roles of Opioid Agonists and IFN-γ on Gut-Derived Sepsis Due to P. aeruginosa as Revealed Using Caenorhabditis elegans and Surgically Stressed Mice

The data provide strong evidence that opioid agonists and IFN-γ enhance the virulence of P. aeruginosa in vitro through the action of PA-I. Yet the degree to which opioid agonists and IFN-γ influence the in vivo lethality of P. aeruginosa is unknown. Thus, the ability of opioids and IFN-γ to enhance the in vivo lethality of P. aeruginosa is assessed, e.g., in two complementary animal models.

Wild-type N2 Caenorhabditis elegans worms are grown to the L4 larval stage on normal growth medium (NGM) with E. coli OP50 as a nutrient source. Specialized agar plates are prepared onto which the PA-I-inducing compounds (vehicle (negative control), opoids (morphine, κ and δ agonist), IFN-γ, and C4-HSL (positive control)) will be added and adsorbed into the agar as described for ethanol⁷⁹. The ability to embed bioactive compounds into the C. elegans growth agar is well described⁸⁰⁻⁸⁶. Lawns of P. aeruginosa (wild type PAO1 and PA-I knockout PAO1 (lecA-)) are then grown on solid at agar plates by adding cultures of P. aeruginosa previously grown overnight in liquid media. Worms from the NGM medium are transferred onto the prepared culture dishes and killing dynamics assessed over time at temperature conditions of 25° C. Experiments are performed at different doses and re-dosing schedules to establish the optimum conditions under which a killing effect for each of the PA-I-inducing compounds can be demonstrated.

To test the ability of PA-I inducing compounds to enhance the lethality of P. aeruginosa in the established mouse model of gut-derived sepsis, mice are fasted for 24 hours and are subjected to general anesthesia, a 30% surgical hepatectomy, and cecal instillation of 106 cfu/ml of wild-type PAO1 or PAO1 (lecA-) via direct puncture. Dose-response curves for P. aeruginosa in this model have been established and show that 10⁶ cfu/ml of P. aeruginosa induces a 50% mortality rate at 48 hours. In order to demonstrate that opioid agonists or IFN-γ enhance the lethality of P. aeruginosa in this model, varying doses of each are suspended in 1 ml of 0.9% NaCl and injected retrograde into the ileum in order to provide a constant supply of the PA-I-inducing compound for 24 hours. Normal saline alone is used for controls. This maneuver is known to be efficacious in delivering a continuous supply of an exogenous compound to the cecum in this model. Mice are fed water only for the next 24-48 hours and mortality recorded. Mice that appear moribund are sacrificed and the cecal mucosa, liver, and blood are cultured for P. aeruginosa growth on Pseudomonas isolation agar (PIA) in order to quantify bacterial adherence and dissemination patterns. The mice used in the study include two strains (wild-type+PA-I knockout) and, with 6 groups of 10 mice per group, a total of 120 mice is suitable.

Increased mortality in worms feeding on lawns of P. aeruginosa in the presence of opioids and/or IFN-γ demonstrates the ability of these compounds to induce a lethal phenotype in this organism against the intestinal epithelium. The demonstration of enhanced killing of worms in these experiments also serves to establish the feasibility and applicability of this model. As disclosed herein, in the absence of PA-I-inducing compounds, C. elegans displays a 50% mortality rate at 80 hours. In testing opioids and/or IFN-γ, or in screening for modulators of PA-I lectin/adhesin activity in general, it should be noted that, following 48 hours of growth and reproduction, worms can reproduce and progeny worms can be indistinguishable from the parent worms and overgrow the plates. If killing dynamics in response to PA-I-inducing compounds are such that observations extend past 48 hours, then use of a temperature sensitive mutant, e.g., C. elegans GLP4 (which does not reproduce at 25° C.), is preferred. Complementary experiments in mice will verify results obtained with worms.

The use of mouse studies to confirm results obtained with C. elegans preferably includes verification that luminally delivered PA-I-inducing compounds are efficacious in up-regulating PA-I as a general measure of enhanced virulence. To control for this possibility, experiments are performed to show that the PA-I-inducing compounds injected into the small bowel enhance PA-I expression in the mouse cecum. One approach involves the use of quantitative RT-PCR for PA-I and exotoxin A on freshly isolated RNA from cecal contents 24 hours following cecal instillation of P. aeruginosa (see FIG. 6). An alternative approach to delivering opioids and IFN-γ directly into the cecum is to engineer non-pathogenic E. coli strains that produce both morphine and IFN-γ. The feasibility of making recombinant morphine and IFN-γ in E. coli is well documented . Mice subjected to a surgical stress (e.g., hepatectomy) are then co-inoculated directly into the cecum with the LD₅₀ dose of P. aeruginosa (approximately 10⁶) and the morphine- and/or IFN-γ-producing E. coli strain. In this manner, P. aeruginosa would be directly exposed to a constant supply of the PA-I-inducing compound such as might naturally occur in vivo. Relevant here is the knowledge in the art that numerous microbial strains (E. coli, Pseudomonas, Candida) naturally produce opioids, especially morphin^{87,89}. In addition, the “microbial soup” typical of a critically ill patient consists of highly pathogenic and resistant strains of bacteria that compete for nutrients in a highly adverse environment. Therefore, not only will the use of morphine- and/or IFN-γ-producing E. coli constitute a feasible alternative approach to obtaining in vivo mouse data, it may also recapitulate actual events in vivo. Finally, C. elegans normally feed on E. coli strains that do not induce mortality. The availability of morphine- and/or IFN-γ-producing E. coli strains could also be used in the C. elegans assays. Others have shown the feasibility of this approach is feasible in mice, as shown by delivering IL-10 into the intestinal mucosa of mice using direct intestinal instillation of bacteria that produce recombinant IL-10⁹⁰. The use of the C. elegans assay is expected to result in the rapid identification of therapeutics and prophylactics that modulate expression of a virulence phenotype by microbial pathogens in contact with, or proximity to, a mammal. The virulence phenotype is amenable to assessment using a variety of measures, many of them indirect, e.g., measurement of epithelial cell barrier function.

### Example 19

Opioids and/or IFN-γ Release into the Intestinal Lumen Resulting from Host Stress

Endogenous morphine concentrations in the blood of humans and animals increase in direct response to the degree of surgical stress⁵⁰. The neural network of the mammalian intestine contains the most abundant concentration of opioid receptors in the body91. Morphine has been recently shown to enhance the release of nitric oxide in the mammalian gastrointestinal tract via the μ3 opiate receptor subtype⁶⁴. In addition, it has been shown that the nematode, Ascaris suum, produces and liberates morphine in the gut⁹². Similarly, IFN-γ has been shown to be released by the gut from intestinal intraepithelial lymphocytes in response to a variety of stressors, including bacterial challenge⁹³ and ischemia/reperfusion injury (I/R)⁹⁴.

To demonstrate that C. elegans produces or releases morphine, worms are grown permissively at 20° C. in massive cultures in liquid medium to 1×10⁶ worms using conventional culturing techniques. Stock cultures are treated with antibiotics 24 hours prior to the imposition of stress conditions. Worms are separated from any remaining bacteria by sedimentation and sucrose flotation as known in the art. Worms are then exposed to either heat stress (35° C. for 1 hour) followed by 2 hours of recovery, or hypoxic stress (0.3% O₂ for 45 minutes) followed by 1 hour of normoxic recovery, as described⁹⁵. Control worms are maintained at 20° C. and 21% O₂. Both the growth medium and the supernatant of homogenized C. elegans are preferably assayed for morphine by HPLC/GC/MS using conventional techniques. To determine whether morphine and IFN-γ are produced by, or released into, the mouse intestine following surgical stress, groups of mice (n-10/group) are subjected to a 30% hepatectomy or segmental mesenteric ischemia as described below. Surgical stress involving the hepatectomy model consists of performing a 30% surgical hepatectomy or sham laparotomy for controls and 24 hours later by harvesting the cecal tissue, the cecal luminal contents, and blood for morphine and IFN-γ assays. The ischemia reperfusion model (I/R) involves isolation of a 10 cm segment of distal ileum that is luminally cannulated and subjected to 10 minutes of ischemia (segmental artery clamp) followed by 10 minutes of reperfusion. Luminal perfusion with 2 ml of Ringers solution is performed to collect the luminal contents before and after I/R. Luminal contents, the homogenized intestinal segment, and blood are assayed for morphine by HPLC and GC/MS; IFN-γ is assayed by ELISA using a specific anti-IFN-γ antibody. A suitable number of mice for such assays is 30-50 mice.

Release of significant amounts of morphine and/or IFN-γ into the gut following surgical stress confirms that P. aeruginosa has been exposed to highly active compounds capable of activating or enhancing its virulence phenotype during host stress. In addition, a better understanding of the precise concentration of morphine and/or IFN-γ to which P. aeruginosa are exposed in vivo can be determined by these experiments. Whether morphine is released in high concentration in the lumen versus within the intestinal tissues is amenable to experimental determination. If luminal levels of morphine are elevated in hepatectomy versus controls, mice can be decontaminated with antibiotics (e.g., ciprofloxacin, metronidazole). Following such decontamination, the extent to which the luminal flora contribute to the opioid level can be determined using conventional techniques. It should be noted that, in addition to, e.g., morphine, other opioids and cytokines may be released from microbial pathogens such as P. aeruginosa that actively participate as host stress-derived BSCs. It is also possible that both opioids and IFN-γ are enzymatically degraded in the intestinal lumen. An alternative approach would be to use quantitative immuno-fluorescence of stained tissues to assess morphine and IFN-γ presence in tissues as antibodies specifically recognizing these compounds are readily available. Notwithstanding the preceding observations, these compounds have been measured by others from luminal contents without difficulty.

### Example 20

Use of Knockout Mice to Confirm the Role of BSCs on PA-I Lectin/Adhesin Activity

IFN-γ is a key immune element that actively participates in both the local and systemic clearance of bacteria during acute infection⁹⁶. Animal models have shown that IFN-γ knockout mice have higher mortality rates following infectious challenge at local tissue sites (lung) compared to IFN-γ-sufficient mice in association with diminished ability to clear bacteria^{94,96-100}. Virtually all of the studies that have assessed the role of IFN-γ on P. aeruginosa infection in vivo have been performed in non-stressed mice where the infectious challenge has been instilled into the lung, and not in stressed mice, such as surgically stressed mice.

The lethality of intestinal P. aeruginosa is tested in IFN-γ knockout mice and wild-type controls (n=10 each group) in an established model of gut-derived sepsis. Mice fasted for 24 hours undergo 30% surgical hepatectomies followed by instillation of 10⁶ cfu/ml of wild type PAO1 into each cecum via direct puncture. Mice are then allowed water only for the remainder of the experiment and mortality is followed for 48 hours. Mice that appear moribund are sacrificed and the cecal mucosa, liver, and blood is quantitatively cultured on Pseudomonas isolation agar (PIA) to determine the rates of bacterial adherence and dissemination. To determine if PA-I expression in P. aeruginosa is attenuated in IFN-γ, a GFP PA-I reporter strain is injected directly into the cecum of mice subjected to a 30% hepatectomy and bacterial strains are recovered 24 hours later to determine fluorescence, as described in FIG. 5. The results of these experiments guide the performance of complementary studies using the segmental mesenteric ischemia model, as depicted in FIG. 7. Briefly, the lumena of 10 cm ileal segments subjected to sham ischemia (no clamp), 10 minutes of ischemia, and 10 minutes of reperfusion is perfused with Ringers solution and the timed aliquots of the perfusates is collected from both IFN-γ knockout mice and their wild-type cohorts. Use of the GFP-PA-I reporter strains facilitates the determination of the extent to which each perfusate induces PA-I promoter activity. A suitable number of mice for such studies is 50 mice, divided into five groups with ten mice in each group.

The display of attenuated lethality by P. aeruginosa in IFN-γ knockout mice is consistent with IFN-γ playing a role as a host stress-derived bacterial signaling compound, or protein, during stress (e.g., surgical stress). IFN-γ may be only one of many signals necessary to orchestrate a fully lethal virulence repertoire for P. aeruginosa under the circumstances of catabolic stress, however. It is noted that IFN-γ knockout mice subjected to hepatectomy may develop an overcompensated and excessive inflammatory response to intestinal P. aeruginosa, resulting in increased mortality that is based more on immune response than enhanced microbial virulence. Tissue and blood culture results from these studies are used to determine whether mortality is due, in part, to such overcompensation. An alternative approach to distinguish between these possibilities is to perform studies in IFN-γ knockout mice and their matched wild-type cohorts (with and without surgical hepatectomy) to determine if there is a mortality difference when groups of mice are systemically inoculated (e.g., intraperitoneal, intravenous, lung instillation) with P. aeruginosa.

### Example 21

Screens for Stress-Induced Bacterial Signaling Compounds

The data disclosed herein establishes that i) filtered luminal contents from the cecum of mice subjected to hepatectomy, or from the small bowel lumen of intestinal segments subjected to mesenteric arterial occlusion, induce a strong signal in P. aeruginosa to express PA-I; and ii) media and membrane preparations from hypoxic or heat-shocked Caco-2 cells induce PA-I expression.

A. Stress-Derived BSCs that are Present in the Media of Caco-2 Cells Exposed to Ischemia and Heat Shock Stress and that Induce PA-I Expression in P. aeruginosa

Intestinal epithelial hypoxia is a common consequence of critical illness following surgical stress and is often an inadvertent consequence of its treatment. In addition, hyperthermia often develops during the acute stress response to injury and infection. Disclosed herein are data demonstrating that hypoxic or hyperthermic stress to cultured intestinal epithelial cells (Caco-2) causes the release of soluble PA-I-inducing compounds into the cell culture medium. This example discloses the isolation and identification of PA-I-inducing compounds that are released by Caco-2 cells exposed to hypoxia and hyperthermic stress.

Two sets of experiments are preferably performed. In the first set of experiments, Caco-2 cells grown to confluence in cell culture plates (150 cm²) are exposed to either normoxia (21% O₂,) or hypoxia (0.3% O₂ for 2 hours followed by 1 hour of normoxic recovery). In the second set of experiments Caco-2 cells are exposed to normothermic (37° C.) or hyperthermic (immersed in water bath at 42° C. for 23 minutes followed by 3 hours recovery) conditions. Paired samples from each set of experiments are then processed to identify the specific host stress-derived bacterial signaling compound(s) using GFP-PA-I reporter strains as a detection system. Media from Caco-2 cells is collected, filtered through a 0.221 μm filter (Millipore) and separated by molecular weight using centricones with a MW cutoff of 3, 10, 30, 50, 100 KDa (<3, 3-10, 10-30, 30-50, 50-100, >100 KDa). All fractions are preferably tested in 96 well plates to determine fractions that activate PA-I expression using PA-I GFP reporter strains. Two preferred approaches are contemplated for use in identifying the proteins that activate PA-I in the stress-conditioned media (hypoxia, hyperthermia). The first approach subjects bioactive fractions (i.e those that induce PA-I), and their molecular weight-matched control fractions (non-PA-I-inducing), to Maldi-Mass Spectrometry (MS) analysis. Spectra from the control media fractions are compared to the fractions of stress-conditioned media to determine the appearance of possible protein molecular ions present only in the samples that induce PA-I. This will allow us to subtract proteins that are present in both non-PA-I-inducing and PA-I-inducing fractions. In order to separate the molecular ion protein peaks that are present only in the PA-I-inducing fractions, bioactive fractions are loaded onto an HPLC equipped with a Vydac C4 column. Eluted samples are collected as fractions and individual fractions are tested for the ability to induce PA-I expression using the GPF-PA-I reporter strain. Proteins are then further separated, preferably by MW, hydrophobicity, and charge using stepwise well-controlled physico-chemical separation techniques in the HPLC system. Samples pre-fractionated in this manner should simplify the observed mass spectra and increase the likelihood of observing any putative protein(s) that induce PA-I expression. For any such proteins, identification using bottom-up proteomics techniques is performed.

An alternative to the use of molecular ion spectra, suitable in studies presenting highly complex spectra, is the classical approach for protein purification using conventional techniques such as ion exchange, hydrophobic, size exclusion, and/or affinity chromatography. Purification of host stress-derived BSCs is preferably assessed using the GFP-PA-I reporter strain.

For protein identification, protein-containing fractions are digested by using trypsin and digested fractions are analyzed with a LC/MSD XCT ion trap mass spectrometer system (Agilent Technologies, Santa Clara, Calif.). Data analysis for the data from the mass spectrometer is carried out using the SpectrumMill software platform (Agilent Technologies, Santa Clara, Calif.). Confirmation of the ability of identified proteins to induce PA-I expression is conveniently achieved in the PA-I:EGFP reporter strain by measuring fluorescence, and in P. aeruginosa strain PAO1 by immunoblot analysis.

Two protein fractions from Caco-2 cells that induce PA-I expression have been identified. Identification of specific active proteins (i.e., epithelial cell-derived PA-I signaling proteins) within the fraction(s) is achieved using any known technique, and preferably using a proteomics facility such as the University of Chicago proteomics facility. Many of these proteins may originate from the cell membranes themselves, since the most potent induction of PA-I expression occurs following contact with an epithelial cell membrane. In addition to protein identification, antibodies specifically recognizing such proteins are contemplated for such uses as cellular (e.g., Caco 2) localization studies. Although there are more classical approaches to protein identification, mass spectrometry is the most cost effective and rapid approach. For non-proteinaceous PA-I inducing compounds, lipid assays are contemplated that involve adjusting fraction pH to 3.5, followed by HPLC using, e.g., a Sep-Pak C₁₈ column. Eluted samples are trapped on a fraction collector, evaporated to dryness, and re-suspended in PBS for PA-I reporter assays. The structure of the active compound is preferably identified with IT/LC/MS/MS. For bacterial signaling compounds that are neither protein nor lipid, relevant fractions are resolved by IT/LC/MS/MS using a C₁₈ column and a quadrapole-time of flight mass spectrometer and NMR. Individual compounds are determined by their mass-fragmentation spectra, isolated, and tested for PA-I inducing activity using GFP reporter strains. Alternative approaches, such as 2D-SDS-PAGE electrophoresis for protein separation and TLC for non-protein separation, are also contemplated. Proteins separated by 2D-SDS-PAGE are typically transferred to a polyvinylidene difluoride transfer protein membrane for automated Edman degradation N-terminal sequence determination using an ABI 477A protein sequencer (Applied Biosystems). Protein identification is further facilitated by sequence comparison to database(s).

In addition to the foregoing screens for modulators, the invention contemplates any assay for a modulator of the expression of a virulence phenotype by a microbe in association with, or proximity to, a mammal such as a human. In particular, the invention comprehends a wide variety of assays for modulators of, e.g., eukaryotic cell barrier fuiction, such as epithelial cell barrier function (e.g., epithelial cells of the intestine, lung, and the like). The invention further comprehends numerous assays for modulators of PA-I lectin/adhesin activity, whether due to a modulation of the specific activity of PA-I or a modulation of the expression of PA-I of constant specific activity, or both. In general, the invention contemplates any assay known in the art as useful for identifying compounds and/or compositions having at least one of the above-described characteristics.

### Example 22

Miscellaneous Methods

A. Screens for PA-I Modulators Using a PA-I Reporter Construct

Media from Caco-2 cells exposed to either hypoxia or heat shock stress induced PA-I expression in P. aeruginosa. Candidate PA-I inducer compounds that are released into the extracellular milieu following epithelial stress include ATP, lactate, cAMP, cytokines, and heat shock proteins.

The aforementioned candidate modulators, and other candidate modulators found in properly conditioned media, are identified using screening methods that constitute another aspect of the invention. Screens for such modulators are conveniently conducted in 96-well plates that contain the GFP-PA-I reporter strain PA27853/PLL-EGFP (see Example 1). The reporter strain is exposed to varying concentrations of candidate host stress BSCs including, but not limited to, heat shock proteins (HSP 25, 72, 90, 110), extracellular nucleosides and nucleotides (adenosine, ATP, cAMP) and cytokines (IL-1-18). Agents are added to the wells and dynamic assessment of bacterial fluorescence is carried out over 12 hours. Positive results are preferably verified by Western blot analysis of PA-I expression. For proteins that induce a PA-I response, the invention further comprehends assays to identify the receptors on P. aeruginosa to which such proteins bind. In one embodiment of this aspect of the invention, the identified protein inducer of PA-I activity is used as a probe to screen, e.g., a comprehensive library of P. aeruginosa by dot blot analysis. Confirmation of the screen results is available by assaying the protein-binding capacity of a lysate from a corresponding clone from a P. aeruginosa transposon library in which the relevant coding region has been disrupted by insertional inactivation.

Identified modulators are then subjected to additional in vitro and in vivo virulence assays to refine the understanding of the role in virulence expression played by such modulators.

B. Caco-2 and MDCK Cell Culture, Measurement of TEER and Exotoxin A Flux

Caco-2 cells and MDCK cells are well-differentiated epithelial cell lines that maintain a stable TEER when grown in confluent monolayer. Apical to basolateral exotoxin A flux across monolayers is assessed with Alexa 594 labeled exotoxin A using standard flux measurements.

C. Bacterial Strains

P. aeruginosa strain PAO1 was obtained from the University of Washington Genome Center and is preferably used in the procedures disclosed herein, where appropriate.

D. Caenorhabditis elegans Assays

Use of the nematode to assay for the lethality of P. aeruginosa is accomplished using standard protocols, as described herein.

E. Antibodies

Antibodies to PA-I are generated using conventional techniques. Preferably, such antibodies are purified by affinity chromatography. IFN-γ and morphine antibodies are commercially available.

F. Dot Blot Assays for Membrane Binding

ImmunoDot Blot assays for the detection of bacterial proteins in large matrix systems have been described elsewhere⁷⁴ and are known in the art. The technique has been validated as highly sensitive and accurate.

G. Transcriptome Analysis of Bacterial Strain PAO1

RNA is isolated from bacterial cultures exposed to opioids and/or IFN-γ as described herein at optical densities of 0.5, 1.0, 2.0. Between 1×10⁹ and 2×10⁹ cells are then mixed with RNA Protect Bacteria reagent (Qiagen) and treated as recommended by the manufacturer's mechanical disruption and lysis protocol. RNA is purified by using RNeasy mini columns (Qiagen), including the on-column DNase I digestion described by the manufacturer. In addition, the eluted RNA is preferably treated for 1 hour at 37° C. with DNase I (0.1 U per μg of RNA). DNase I is then removed by using DNA-Free (Ambion) or by RNeasy column purification. RNA integrity is monitored by agarose gel electrophoresis of glyoxylated samples. Further sample preparation and processing of the P. aeruginosa GeneChip genome arrays are then done as described by the manufacturer (Affymetrix). For cDNA synthesis 12 μg of purified RNA is preferably combined with semirandom hexamer primers with an average G+C content of 75%, and Superscript II reverse transcriptase (Life Technologies). Control RNAs from yeast, Arabidopsis, and Bacillus subtilis genes are added to the reaction mixtures to monitor assay performance. Probes for these transcripts are tiled on the GeneChip arrays. RNA is removed from the PCR mixtures by alkaline hydrolysis. The cDNA synthesis products are purified and fragmented by brief incubation with DNase I, and the 3′ termini of the fragmentation products are labeled with biotin-ddUTP. Fragmented and labeled cDNA is hybridized to an array by overnight incubation at 50° C. Washing, staining, and scanning of microarrays is performed with an Affymetrix fluidic station.

H. Analysis of Expression Profiling

The Affymetrix Microarray Software suite (MAS) (version 5.0) is a suitable software choice for determining transcript levels and whether there are differences in transcript levels when different samples are compared. Affymetrix scaling is used to normalize data from different arrays. A scale factor is derived from the mean signal of all of the probe sets on an array and a user-defined target signal. The signal from each individual probe set is multiplied by this scale factor. For any given array, between 18 and 28% of the mRNAs are considered absent by MAS, indicating that the corresponding genes are not expressed at levels above background levels. Furthermore, it is known in the art that the average changes in control transcript intensities are less than twofold for any comparison of array data. This indicates that the efficiency of cDNA synthesis and labeling is similar from sample to sample. For comparative analyses, the log₂ ratio for-absolute transcript signals obtained from a given pair of arrays will be calculated by using MAS. A statistical algorithm of the software is also assigned a change call for each transcript pair, which indicates whether the level of a transcript is significantly increased, decreased, or not changed compared to the level for the baseline sample. The baseline samples are those derived from cultures of P. aeruginosa PAO-1 without any added opioids or IFN-γ. Graphical analyses of the signal log ratios from each experiment (any pair of arrays) is performed to display a normal distribution with a mean very close to zero (no change). Among the transcripts with significant increases or decreases compared to the baseline in one or more samples, those that showed at least a 2.5-fold change are subjected to further analysis. For cluster analyses and transcript pattern analyses, GeneSpring software (Silicon Genetics, Redwood City, Calif.) is contemplated as a suitable choice. The fold change values for each gene will be normalized independently by defining the half-maximal value for the gene as 1 and representing all other values as a ratio that includes that value. This scaling procedure will allow direct visual comparison of gene expression patterns within an experiment, as well as between experiments. GeneSpring is also contemplated for use in sorting genes according to the P. aeruginosa genome project.

I. Solubilization of Non-Denatured and Denatured Membrane Proteins Fractions from P. aeruginosa

P. aeruginosa cells are washed with PBS and re-suspended in PBS containing a protein inhibitor cocktail. For preparation of membrane fractions, P. aeruginosa cells are disrupted by French pressure and centrifuged at 10000 g×30 minutes to eliminate debris. The supernatant is recentrifuged at 50000 g×60 minutes. The pellet is solubilized in 4% CHAPS at 37° C. for 3 hours. After being recentrifuged at 50000 g×60 minutes, the supernatant is spun through a 100K centricone and dialyzed against PBS. The binding capacity of the solubilized protein to γ-IFN is confirmed by ELISA binding assay.

J. Statistical Analysis and Protein-Protein Interactions

For statistical analysis, all data are preferably loaded into the SigmaStat platform software and appropriate tests applied. Protein-protein interaction studies are performed using conventional protocols, as would be known in the art.

K. Maldi-MS Analysis

Samples (0.5 μL) are mixed with an equal volume of a 5 mg/mL solution of α-cyanohydroxycinnamic acid in 30% acetonitrile in water with 0.1% TFA and are then manually spotted onto a 192 spot target plate (Applied Biosystems, Foster City, Calif.). The plate is inserted into a 4700 MALDI TOF/TOF (Applied Biosystems, Foster City, Calif.) operated in linear mode. Samples are desorbed by a 200 Hz YAG laser. The acquisition program is set to acquire a summed spectrum (200-1000) shots across the range 5000 to 100000 Thompsons.

L. Digestion of a Protein Containing Fraction by Using Trypsin to Prepare for Protein Identification

The protein extract sample is diluted in 50 mM ammonium carbonate buffer, pH 8.5, containing 0.1% Rapigest SF acid labile detergent (Waters Corp, Millford, Mass.). The sample is heated to 100° C. for 10 minutes to completely denature the proteins. Ten μL of 10 mM TCEP is added to reduce disulfide bonds and the sample is incubated for 10 minutes at 37° C. The pooled sample is digested with Lys-C (12.5 ng/μL) at a mass ratio of 1:100 for 3 hours at 37° C. and then digested with trypsin (12.5 ng/μL) at a mass ratio of 1:50 (trypsin:protein) for 3 hours at 37° C. Digestion is halted by adding PMSF to final concentration of 1 mM. After digestion,10 μL of TFA is added to the solution and the sample is incubated for 45 minutes at 37° C. to destroy the acid labile Rapigest detergent.

M. LCIMSD XCT Ion Trap Mass Spectrometry Analysis

A digested protein sample is injected (10 μL) onto an HPLC (Agilent Technologies 1100) containing a C 18 trapping column (Agilent Technologies, Santa Clara, Calif.) containing Zorbax 300SB-C18 (5×0.3 mm). The colum valve is switched to its secondary position 5 minutes after injection and the trapped peptides are then eluted onto a 75 μm id Zorbax Stablebond (300 A pore) column and chromatographed using a binary solvent system consisting of A: 0.1% formic acid and 5% acetonitrile and B: 0.1% formic acid and 100% acetonitrile at a flow rate of 300 nL/minute. A gradient is run from 15% B to 55% B over 60 minutes on a reversed-phase column (75 μm id Zorbax Stablebond (300 A pore), and the eluting peptides are sprayed into a LC/MSD XCT ion trap mass spectrometer system (Agilent Technologies, Santa Clara, Calif.), equipped with an orthogonal nanospray ESI interface. The mass spectrometer is operated in positive ion mode with the trap set to data dependent auto-MS/MS acquisition mode. Source conditions are: Vcap −4500V, drying gas flow 8 Uminute, drying gas temperature 230° C. and CapEx 65V. The instrument is set to complete a mass scan from 400-2200 Thompsons in one second. Peaks eluting from the LC column that have ions above 100,000 arbitrary intensity units trigger the ion trap to isolate the ion and perform an MS/MS experiment scan after the MS full scan. The instrument's dynamic ion exclusion filter is set to allow the instrument to record up to 2 MS/MS spectra for each detected ion to maximize the acquisition of qualitative data from peptides (by preventing high abundance peptides from dominating the subsequent MS/MS experiments) and the excitation energy is set to “smart frag” mode to insure the generation of useful product ion spectra from all peptides detected. Data files that result are then transferred to a file server for subsequent data reduction.

N. The Mass Spectrometer Data Analysis with the SpectrumMill Software Platform

SpectrumMill is derived from the MS-Tag software package originally developed by Karl Clauser et al.¹⁰¹and is contemplated as a suitable software platform. Raw data is extracted from the MS data files using the data extractor module and the data is then subjected to protein library search and de Novo spectral interpretation by the Sherenga module¹⁰². SpectrumMill is designed to minimize spurious identifications obtained from the MS/MS spectra of peptides by careful filtering and grouping of related MS and MS/MS data during extraction from the raw data file. The library searching and de Novo interpretation identify the detected proteins form the individual peptides. The results for all proteins detected are collected and listed by protein name, detected peptide sequence(s), and search score. The reports are exported to an Excel spreadsheet file for inclusion in a result database. In addition, data extracted from the raw data files from the ion trap are preferably submitted to the Mascot (MatrixScience Inc, London, UK) search program and searched against both the NCBI non-redundant protein database and the SWISSPROT protein database. The identifications from these two systems are correlated to arrive at a final consensus list of identified proteins.

O. Separation of Lipid Fractions on HPLC System

Fractions are pH adjusted to 3.5, and run across a Sep-Pak C₁₈ column on a HPLC system (Millipore corp., Milford, Mass.). The columns are washed with ddH₂O, and compounds are eluted with increasingly polar mobile phases (hexane-methyl formate-methanol). Fractions are concentrated under a stream of nitrogen and reconstituted in either 1 ml PBS (for PA-I reporter assay) or 100 ul of methanol (for UV/HPLC). Active fractions from Sep-Pak are preferably further resolved by a C₁₈ reversed-phase HPLC column (150 mm×5 mm, Phenomenex, Torrance, Calif.) with acidified (0.1% acetic acid) MetOH:H₂O (60:40 vol/vol) at 1 ml/minute on a 1050 series HPLC using ChemStation™ software (Hewlett Packard, Palo Alto, Calif.).

### Example 23

Hypoxia-Induced PA-Lectin Adhesin Expression

The aim of the study described in this Example was to determine whether intestinal epithelial hypoxia, a common response to surgical stress, could activate PA-I expression. Because splanchnic vasosconstriction and intestinal epithelial hypoxia are a common consequence of surgical injury, the aim of the experiments described herein was to determine the specific role of the intestinal epithelium in signaling to P. aeruginosa by examining the effect of epithelial cell hypoxia and reoxygenation on PA-I expression. A fusion construct was generated to express green fluorescent protein downstream of the PA-I gene, serving as a stable reporter strain for PA-I expression in P. aeruginosa, as described in Example 1. Polarized Caco-2 monolayers were exposed to ambient hypoxia (0.1-0.3% O₂) for 1 hour, with or without a recovery period of normoxia (21% O₂) for 2 hours, and then inoculated with P. aeruginosa containing the PA-I reporter construct. Hypoxic Caco-2 monolayers caused a significant increase in PA-I promoter activity relative to normoxic monolayers (165% at 1 h; P<0.001). Similar activation of PA-I was also induced by cell-free apical, but not basal, media from hypoxic Caco-2 monolayers. PA-I promoter activation was preferentially enhanced in bacterial cells that physically interacted with hypoxic epithelia. As shown below, the virulence circuitry of P. aeruginosa is activated by both soluble and contact-mediated elements of the intestinal epithelium during hypoxia and normoxic recovery.

Human Epithelial Cells

Caco-2_{BBe} cells expressing SGLT1 were maintained in DMEM with 25 mM glucose (high-glucose DMEM) with 10% fetal calf serum, 15 mM HEPES, pH 7.4, and 0.25 mg/ml geneticin, as previously described (17). Caco-2 cells were plated on 0.33-cm² collagen-coated, 0.4-μm pore size polycarbonate membrane Transwell supports (Corning-Costar, Acton, Mass.) for 20 days, and media were replaced with identical media without geneticin at least 24 hours before bacterial inoculation.

GFP Fusion Constructs of Wild-Type P. aeruginosa

P. aeruginosa (ATCC-27853, American Type Culture Collection, Manassas, Va.) was transformed with the plasmid pUCP24/PLL-EGFP. This construct harbors a PA27853 chromosomal DNA fragment containing an upstream regulatory region of PA-I followed by the entire PA-I gene fused at the COOH terminal with an enhanced green fluorescent protein (EGFP) gene excised from the pBI-EGFP plasmid (Clontech, Palo Alto, Calif.). Expression of the PA-I lectin was detected by fluorescence microscopy and fluorimetry of this reporter strain as previously described (21).

Dynamic Fluorimetry

Caco-2 cells were grown to confluence on collagen-coated 96-well fluorimetry plates (Becton Dickinson Labware, Bedford, Mass.) and maintained in a 37° C. incubator with 5% CO₂ and 21% O2. The day before experiments, media were removed and replaced with 150 μl of antibiotic-free media. Three experimental conditions were created using a modification of the methods previously described by Xu et al. (23). In control conditions, Caco-2 cells were maintained in a 5% CO2-21% O2 incubator for 2 hours. Hypoxic conditions were achieved by placing Caco-2 cells in a humidified hypoxia chamber at 37° C. with 5% CO-95% N2 for 2 hours. Measured 02 in the chambers varied between 0.1 and 0.3%. To simulate a reperfusion or reoxygenation state (normoxic recovery), after 2 hours of Caco-2 cell hypoxia, hypoxic media were completely replaced with fresh, normoxic HDMEM media, and the cells were allowed to recover under normoxia (37° C., 5% CO₂-21% O2) for 2 hours before bacterial inoculation. The fluorescent reporter strain PA27853/PLL-EGFP was next added to the three groups of Caco-2 cells. Bacteria were cultured overnight in Luria-Bertani broth containing 20 μg/ml gentamicin at 37° C. under shaking conditions. After about 12 hours of growth, 50 μl of the bacterial suspension were added to the 96-well plates of Caco-2 cells. Care was taken to ensure that all bacterial samples were cultured for identical periods of time and that wells contained equal cell densities. Fluorescence was tracked immediately following bacterial inoculation and then hourly thereafter up to 3 hours using a 96-well microplate fluorimeter (Synergy HT, Biotek, Winooski, Vt.). Plates were maintained in standard incubators at 37° C. with 5% CO₂-21% O₂ between all measurements. Fluorescence values were calculated as follows: %control=100×(RFUxₜ₌ₙ−RFUxₜ₌₀)/(RFUcₜ₌ₙ−RFUcₜ₌₀), where RFUx refers to the hypoxic or normoxic recovery groups and RFUc refers to the control at time “n.”

Exposure of Bacteria to Filtered Media from Caco-2 Cells and Potential PA-I-Inducing Candidate Molecules

In this set of experiments, reiterative conditions of control, hypoxia, and normoxic recovery (i.e., reperfusion/reoxygenation) were created in 96-well plates containing confluent Caco-2 cells. Media from all wells were then collected and passed through a 0.22-μm filter and stored on ice. Ninety-six-well fluorimetry plates without Caco-2 cells (Costar 3631, Corning, Corning, N.Y.) were then prepared by adding a 20-μl bacterial suspension containing overnight growing cultures of PA27853/PLL-EGFP. Media from the three experimental groups were then added to the wells, and fluorescence was assessed over 5 hours, with plates maintained at 37° C. with continuous orbital shaking (100 rpm) between measurements. To screen for potential PA-I-inducing compounds that might be present in the media of hypoxic Caco-2 cell media, purified adenosine, D-lactate, and L-lactate (Sigma-Aldrich, St. Louis, Mo.) were added to wells containing HDMEM across a range of physiologically relevant dosages, which were then tested as described above.

Fluorescence Microscopy

To visually correlate results from the above experiments to the spatiotermporal effects of PA27853/PLLEGFP on hypoxic Caco-2 cells, cells were grown to confluence on Bioptechs dishes (Bioptechs, Butler, Pa.) and exposed to 2 hours of hypoxia followed by inoculation with PA27853/PLL-EGFP. Experiments were performed on a 37° C. microscopy stage and visualized using an inverted fluorescence microscope (Axiovert 100, Carl Zeiss, Thornwood, N.Y.). Z-stacks were collected every 30 minutes for 3 hours. Images were analyzed for bacterial distribution using ImageJ graphics analysis software (Version 1.31, National Institutes of Health, Bethesda, Md.).

Caco-2 Cell Barrier Function During Hypoxia and Normoxic Recovery in the Presence of P. aeruginosa or Purified PA-I

Caco-2 monolayer transepithelial electrical resistance (TER), a measure of barrier function, was assessed using agar bridges and Ag-AgCl-calomel electrodes and a voltage clamp (University of Iowa Bioengineering, Iowa City, Iowa) as previously described (17). TER was calculated using Ohm's law. Fluid resistance was subtracted from all values (17). Two microliters of overnight cultures of PA27853 normalized to cell density or 50 μg of purified PA-I (Sigma-Aldrich) were added to the apical chamber of the Caco-2 cell transwells following exposure to hypoxia and normoxic recovery as detailed above. Caco-2 cell TER was assessed every hour, and cells were maintained at 37° C. with 5% CO₂-21% O₂ throughout the experiment. To determine the effect of PA27853 on the barrier function of Caco-2 cells under conditions of sustained hypoxia, reiterative experiments were performed under continuous hypoxia (37° C., 5% CO₂-95% N₂), in which TER measurements were made every hour for 7 hours within the hypoxic chamber using an EVOM resistance measurement apparatus (World Precision Instruments, Sarasota, Fla.).

Northern Blot Analysis

In selected experiments, PA-I expression was confirmed using Northern blot analyses as previously described (21).

Statistical Analysis

Data were analyzed, and statistical significance was determined using Prism 4.0 (GraphPad Software, San Diego, Calif.). Statistical significance was defined as P<0.05 by Student's t-test or two-way ANOVA, as appropriate.

Results

PA27853/PLL-EGFP P. aeruginosa Respond to the Environment of Caco-2 Cell Hypoxia and Normoxic Recovery with Enhanced Fluorescence

To determine whether the green fluorescent protein (GFP) reporter strain PA27853/PLL-EGFP would display increased PA-I promoter activity when added to Caco-2 cells exposed to hypoxia (2 hours at <0.3% O2) and normoxic recovery (hypoxia followed by 2 hours of recovery in normoxic conditions), reporter strains were added to the media of Caco-2 cells exposed to the two conditions. GFP reporter strains demonstrated significantly more PA-I promoter activity, as measured by fluorescence, within 1 hour of incubation with Caco-2 cells exposed to either hypoxia or normoxic recovery (FIG. 38). The media pH in all experimental conditions was measured at all time points and demonstrated no significant difference among control, hypoxia, and normoxic recovery groups because all media were buffered. However, to show that the pH of media did not influence fluorescence in PA27853/PLL-EGFP, strains were incubated in media at pH 6.5, 7.4, and 7.7. The percent change in fluorescence was not different among groups (6.5=106±10, 7.4=100+12, 7.7=112±12; P=not significant). Similarly, to rule out an effect of hypercarbia or hypoxia alone on PA-I promoter activity in our reporter strains, strains were subjected to hypoxia (0.1% for 2 h) and hypercarbia (80% CO₂ for 2 h). No difference in fluorescence was observed between groups. Taken together, these findings demonstrate that media from Caco-2 cells exposed to hypoxia with or without normoxic recovery activate PA-I promoter activity.

Fluorescence Imaging of GFP Reporter Strains in the Caco-2 Cell Environment

To determine whether epithelial cell contact contributes to the expression of GFP in our PA-I reporter strain, Caco-2 cells were imaged by fluorescent microscopy following exposure to hypoxia and apical inoculation with PA27853/PLL-EGFP. Fluorescence imaging demonstrated that PA27853/PLL-EGFP exposed to hypoxic Caco-2 monolayers appeared markedly more fluorescent than bacteria exposed to normoxic monolayers at the 120-minute time point (FIG. 39). Multiple images of the bacterial/Caco-2 cell coculture demonstrated that more bacteria were located near or within the plane of the cell monolayers exposed to hypoxia than in nonhypoxic cells. Quantitative analysis of multiple microscopy images revealed an average of 658±78 bacteria/high-powered field at the level of the surface of hypoxic epithelia, whereas no bacteria were seen in plane-matched controls (P<0.001).

PA27853/PLL-EGFP Reporter Strains Respond to a Paracrine Factor Present in Media from Caco-2 Cells Exposed to Hypoxia and Normoxic Recovery

To determine whether soluble compounds released into the media in response to Caco-2 cell hypoxia were capable of activating PA-I expression independent of bacterial contact with the epithelium, the ability of media from hypoxic Caco-2 cell cultures to enhance fluorescence in our reporter strain was assessed. PA27853/PLL-EGFP bacteria exposed to filtered media from Caco-2 cells exposed to hypoxia and normoxic recovery developed a significant enhancement of fluorescence that appeared greatest at the 5-hour time point (FIG. 40; control: 3.7%±SD 3.9; hypoxia: 12.6%±SD 5.8; normoxic recovery: 13.1%±SD 3.9; P<0.001 by 2-way repeated measures ANOVA). Results were confirmed by Northern blot analysis (FIG. 40). To determine whether this paracrine factor was isolated to the apical or basolateral compartments, reiterative experiments were performed in which isolated media from the basolateral and apical compartments of hypoxic monolayers, as well as mixtures of apical and basolateral media, were added to wells containing the GFP-PA-I reporter strain PA27853/PLL-EFGP. Only those bacteria exposed to hypoxic media from the apical chamber or hypoxic mixed media showed a statistically significant increase over controls (FIG. 41; P<0.05).

Adenosine Alone Induces PA-I Expression in P. aeruginosa

To determine whether candidate compounds specifically released by hypoxic Caco-2 cells could induce the expression of PA-I, the effect of D-lactate, L-lactate, and adenosine in the GFP-PA-I reporter strains were investigated. D- and L-lactate had no effect on PA-I promoter activity; however, PLL/PA27853 responded with enhanced fluorescence to 10 mM adenosine (FIG. 42), raising the possibility that adenosine released by hypoxic Caco-2 cells could be the putative mediator of the increased PA-I response observed in the above studies. However, the time required for upregulation of PA-I expression was longer than that observed in response to hypoxic cell media, suggesting that other factors may be involved in the signaling pathway.

Caco-2 Cells Exposed to Hypoxia and Normoxic Recovery Resist the Barrier-Dysregulating Effect of Purified PA-I

To determine whether conditions of hypoxia and normoxic recovery enhance or attenuate the barrier-dysregulating properties of PA27853 against Caco-2 cells, TER was measured in Caco-2 cells apically inoculated with either PA27853 (FIG. 43) or purified PA-I (FIG. 44) following exposure to hypoxia and normoxic recovery. Despite the ability of media from hypoxic and reoxygenated Caco-2 cells to increase the expression of PA-I in P. aeruginosa, the TER of Caco-2 cells exposed to these conditions were unchanged in response to a P. aeruginosa inoculated with purified PA-I exhibited an attenuated drop in TER compared with normoxic cells (FIG. 44; P<0.05).

Caco-2 Cells Exposed to Sustained Hypoxia Completely Resist the Barrier Dysregulating Effect of PA27853

To determine whether Caco-2 cells exposed to sustained hypoxia could resist the barrier-dysregulating effect of PA27853, the TER of Caco-2 cells apically inoculated with PA27853 in an environment of sustained hypoxia was measured. Caco-2 cells maintained TER equal to hypoxic Caco-2 cells without bacteria and completely resisted the predicted decrease in TER at the 7-hour time point (FIG. 45). That Caco-2 cells partially resist the barrier-dysregulating effect of strains of PA27853 despite increased PA-I expression is consistent with observations that epithelial cells normally respond to hypoxia with an enhancement of local mucosal defense proteins and barrier function (5).

Soluble Factors Present in the Media of Hypoxic Caco-2 Cells Induce Increased Barrier Resistance in Normoxic Cells

To determine whether the normoxic Caco-2 cells could be induced to increase their resistance to barrier dysregulation by P. aeruginosa through signals present in hypoxic cell media, the apical and basolateral media of normoxic Caco-2 cells was exchanged with filtered media from the apical and basolateral compartments of hypoxic Caco-2 cells and tested the barrier function of these cells when apically inoculated with P. aeruginosa. Normoxic Caco-2 cells exposed to media from hypoxic epithelia displayed a prolonged resistance to barrier dysregulation induced by P. aeruginosa (FIG. 46), leading to the expectation that normoxic epithelia are activated to enhance their barrier function in the presence of soluble mediators produced during hypoxia.

Although P. aeruginosa is not considered to be an intestinal pathogen in the classic sense, it induces one of the most rapid and profound decreases in intestinal epithelial TER of any bacteria reported to date. Previous reports establish that, in both Caco-2 and T-84 cells, P. aeruginosa (PA27853) can induce an 80% decrease in TER within 4 hours following its apical inoculation (9). If defined by this criterion alone, P. aeruginosa is among the most pathogenic organisms to the intestinal epithelium yet described. The observation that as many as 5% of the normal population harbor this pathogen within their intestinal tracts (6), coupled with the animal studies demonstrating that control mice do not develop any symptoms of infection following the direct introduction of large quantities of P. aeruginosa into the cecum (9), indicate that this organism behaves like a classic opportunist, switching virulence genes on and off in response to selected environmental cues. Although it is well established that environmental cues such as pH, redox state, and nutrient composition can activate virulence gene expression in bacteria through a variety of membrane-bound biosensor kinases (24), there have been no previous reports suggesting that bacterial signaling compounds are released by host cells following physiological or ischemic stress. Without wishing to be bound by theory, when viewed from the standpoint of the evolutionary fitness of the microbe, it is logical that a pathogen might recognize the biochemistry of host cell stress, because possessing a system that recognizes host susceptibility would allow for a more accurate assessment of the costs versus benefits of host invasion. Yet, whereas it is well established that intestinal pathogens can communicate directly with the cells to which they adhere, that such a molecular dialogue might be bidirectional is poorly described (7, 15).

To demonstrate that bacteria sense and respond directly to host cells, we used the PA-I lectin/adhesin of P. aeruginosa as a reporter gene. The PA-I lectin is under tight regulatory control of two key systems of virulence gene regulation in P. aeruginosa: the quorum-sensing signaling system and the alternative sigma factor RpoS. The quorum-sensing signaling system and RpoS are interconnected systems of virulence gene regulation in P. aeruginosa that control the expression of hundreds of virulence genes in this pathogen. Because PA-I expression is dependent on the function of both quorum sensing and Rpos, it serves as a relevant biological readout for generalized virulence gene activation in P. aeruginosa (19, 20). The finding that soluble elements of intestinal epithelial cells and, in particular, adenosine can activate PA-I expression, is consistent with specific host cell-derived compounds being released that signal colonizing pathogens such as P. aeruginosa of a weak and susceptible host. That adenosine alone can activate PA-I expression is an important finding given that adenosine is released and can accumulate in the extracellular milieu of hypoxic tissues at high concentrations. During active intestinal inflammation, 5′-AMP derived from migrating polymorphonuclear leukocytes is converted to adenosine by the apical surface epithelium of the intestine. Strohmeier et al. (14) have demonstrated that under normal conditions, the human intestinal epithelial cell line T-84 can convert substantial amounts of 5′-AMP that accumulate to as much as 5 mM adenosine in the apical media within 30 minutes. Although in the present study, activation of PA-I promoter activity in P. aeruginosa required what appeared to be an unphysiological dose of adenosine, the precise concentration of adenosine to which P. aeruginosa might be exposed within the intestinal tract during prolonged hypoxia and reoxygenation is unknown. In addition, adenosine exposure required 6 hours before PA-I promoter activity was observed, whereas with hypoxic media PA-I promoter activity was observed at 4 hours. As a matter of speculation, an opportunistic organism like P. aeruginosa may require an inordinately potent and prolonged host-derived signal for it to invest the resources and energy required to mount a toxic offensive against the intestinal epithelium. Under such circumstances, P. aeruginosa might “sense” that the host on which its survival depends is subjected to an extreme degree of inflammation and vulnerability and hence represents a liability to its survival.

Given that PA-I expression was increased in response to Caco-2 cell hypoxia and normoxic recovery, we expected to see a more profound decrease in TER when P. aeruginosa was apically inoculated onto Caco-2 cells exposed to these conditions. That enhanced PA-I expression in P. aeruginosa did not decrease Caco-2 cell TER during hypoxia could be explained by the enhancing effect of hypoxia itself on Caco-2 cell barrier function. This possibility is supported by the finding that hypoxic media transferred to normoxic Caco-2 cells enhanced their resistance to P. aeruginosa (FIG. 42). This notion is further supported by the finding that hypoxic Caco-2 cells resist the barrier-dysregulating property of purified PA-I, again suggesting that hypoxia enhanced epithelial barrier function to the barrier-dysregulating effects of the PA-I protein of P. aeruginosa. These findings are also in agreement with the known enhancing effect of hypoxia on intestinal epithelial barrier function (8). Furuta and colleagues (5) have demonstrated that exposure of Caco-2 cells to hypoxia increases the expression of both mucin and trefoil peptides, and they have also observed TER to be preserved or even increased in Caco-2 cells during hypoxia. This response makes physiological sense given that under such circumstances, the intestinal epithelial surface will be vulnerable to a potentially hostile flora. However, during reperfusion, which here we have termed normoxic recovery, Caco-2 cells eventually succumb to the potent barrier-dysregulating effect of P. aeruginosa. This is consistent with both clinical and animal studies where the greatest alteration in intestinal permeability and systemic proinflammatory activation occurs during the reperfusion phase following ischemic injury to the intestine (12, 16).

In summary, herein we demonstrate that P. aeruginosa is capable of sensing and responding to local elements of host cell stress. Host-derived bacterial signaling compounds appear to be released by intestinal epithelial cells in response to hypoxia and normoxic recovery, which are often present during critical illness and its treatment. Further elucidation of the precise host compounds or signals that are sensed by colonizing nosocomial pathogens, such as P. aeruginosa, could lead to a better understanding of how infection continues to complicate the course of the most critically ill patients.

### REFERENCES

The citations provided immediately below are exclusively referenced in this Example, and are the only references cited in this Example.
1. Alverdy J, Holbrook C, Rocha F, Seiden L, Wu R L, Musch M, Chang E, Ohman D, and Suh S. Gut-derived sepsis occurs when the right pathogen with the right virulence genes meets the right host: evidence for in vivo virulence expression in Pseudomonas aeruginosa. Ann Surg 232: 480-489, 2000.
2. Alverdy J C and Rocha F. Surgical stress, bacteria, and mucosal immune function. Eur J Pediatr Surg 9: 210-213, 1999.
3. Berkes J, Viswanathan V K, Savkovic S D, and Hecht G. Intestinal epithelial responses to enteric pathogens: effects on the tight junction barrier, ion transport, and inflammation. Gut 52:439-51,2003.
4. Fox J G, Wang T C, Rogers A B, Poutahidis T, Ge Z, Taylor N, Dangler C A, Israel D A, Krishna U, Gaus K, and Peek R M Jr. Host and microbial constituents influence Helicobacter pylori-induced cancer in a murine model of hypergastrinemia. Gastroenterology 124: 1879-1890, 2003.
5. Furuta G T, Turner J R, Taylor C T, Hershberg R M, Comerford K, Narravula S, Podolsky D K, and Colgan S P. Hypoxia-inducible factor 1-dependent induction of intestinal trefoil factor protects barrier function during hypoxia. J Exp Med 193: 1027-1034, 2001.
6. Griffith S J, Nathan C, Selander R K, Chamberlin W, Gordon S, Kabins S, and Weinstein R A. The epidemiology of Pseudomonas aeruginosa in oncology patients in a general hospital. J Infect Dis 160: 1030- 1036, 1989.
7. Gupta R A, Polk D B, Krishna U, Israel D A, Yan F, DuBois R N, and Peek R M Jr. Activation of peroxisome proliferator-activated receptor gamma suppresses nuclear factor kappa B-mediated apoptosis induced by Helicobacter pylori in gastric epithelial cells. J Biol Chem 276: 31059- 31066, 2001.
8. Karhausen J, Ibla J C, and Colgan S P. Implications of hypoxia on mucosal barrier function. Cell Mol Biol (Noisy-le-grand) 49: 77-87, 2003.
9. Laughlin R S, Musch M W, Hollbrook C J, Rocha F M, Chang E B, and Alverdy J C. The key role of Pseudomonas aeruginosa PA-I lectin on experimental gut-derived sepsis. Ann Surg 232:133-142, 2000.
10. Marshall J C, Christou N V, and Meakins J L. The gastrointestinal tract. The “undrained abscess” of multiple organ failure. Ann Surg 218: 111- 119, 1993.
11. Nazli A, Yang P C, Jury J, Howe K, Watson J L, Soderholm J D, Sherman P M, Perdue M H, and McKay D M. Epithelia under metabolic stress perceive commensal bacteria as a threat. Am J Pathol 164: 947-957, 2004.
12. Olanders K, Sun Z, Borjesson A, Dib M, Andersson E, Lasson A, Ohlsson T, and Andersson R. The effect of intestinal ischemia and reperfusion injury on ICAM-1 expression, endothelial barrier function, neutrophil tissue influx, and protease inhibitor levels in rats. Shock 18: 86-92, 2002.
13. Rocha F, Laughlin R, Musch M W, Hendrickson B A, Chang E B, and Alverdy J. Surgical stress shifts the intestinal Escherichia coli population to that of a more adherent phenotype: role in barrier regulation. Surgery 130: 65-73,2001.
14. Strohmeier G R, Lencer W I, Patapoff T W, Thompson L F, Carlson S L, Moe S J, Carnes D K, Mrsny R J, and Madara J L. Surface expression, polarization, and functional significance of CD73 in human intestinal epithelia. J Clin Invest 99: 2588-2601, 1997.
15. Sun J, Hobert M E, Rao A S, Neish A S, and Madara J L. Bacterial activation of -catenin signaling in human epithelia. Am J Physiol Gastrointest Liver Physiol 287: G220-G227, 2004.
16. Sun Z, Lasson A, Olanders K, Deng X, and Andersson R. Gut barrier permeability, reticuloendothelial system function and protease inhibitor levels following intestinal ischaemia and reperfusion- effects of pretreatment with N-acetyl-L-cysteine and indomethacin. Dig Liver Dis 34: 560-569, 2002.
17. Turner J R, Rill B K, Carlson S L, Carnes D, Kemer R, Mrsny R J, and Madara J L. Physiological regulation of epithelial tight junctions is associated with myosin light-chain phosphorylation. Am J Physiol Cell Physiol 273: C1378-C1385, 1997.
18. Viswanathan V K, Sharma R, and Hecht G. Microbes and their products- physiological effects upon mamrnalian mucosa. Adv Drug Delivery Res 56: 727-762, 2004.
19. Winzer K, Falconer C, Garber N C, Diggle S P, Camara M, and Williams P. The Pseudomonas aeruginosa lectins PA-IL and PA-IIL are controlled by quorum sensing and by RpoS. J Bacteriol 182: 6401-6411, 2000.
20. Winzer K and Williams P. Quorum sensing and the regulation of virulence gene expression in pathogenic bacteria. Int J Med Microbiol 291: 131-143, 2001.
21. Wu L, Holbrook C, Zaborina O, Ploplys E, Rocha F, Pelham D, Chang E, Musch M, and Alverdy J. Pseudomonas aeruginosa expresses a lethal virulence determinant, the PA-I lectin/adhesin, in the intestinal tract of a stressed host: the role of epithelia cell contact and molecules of the quorum sensing signaling system. Ann Surg 238: 754-764, 2003.
22. Wu L, Zaborina O, Zaborin A, Chang E B, Musch M, Holbrook C, Shapiro J, Turner J R, Wu G, Lee K Y, and Alverdy J C. Highmolecular-weight polyethylene glycol prevents lethal sepsis due to intestinal Pseudomonas aeruginosa. Gastroenterology 126: 488498, 2004.
23. Xu DZ, Lu Q, Kubicka R, and Deitch E A. The effect of hypoxia/reoxygenation on the cellular function of intestinal epithelial cells. J Trauma 46: 280-285, 1999.
24. Xu J, Chiang H C, Bjursell M K, and Gordon J I. Message from a human gut symbiont: sensitivity is a prerequisite for sharing. Trends Microbiol 12: 21-28, 2004.

### Example 24

Studies have been focused on the interaction between the human opportunistic pathogen Pseudomonas aeruginosa and the intestinal epithelium, and more particularly, in the factors involved in causing P. aeruginosa to activate its virulence against the intestinal epithelium during host stress. It was postulated by the inventors that following stress, soluble factors are released by the host that directly signal Pseudomonas in a manner that activates the virulence of P. aeruginosa through mechanisms that involve the quorum sensing signaling system. Within the quorum sensing regulon, the inventors have identified that expression of PA-I, a lectin adhesion, can open up tight junctions leading to exotoxin A and elastase permeation across the epithelium causing lethal sepsis (see FIG. 25-1 in Appendix A).

Previously it was demonstrated that the PA-I lectin plays a key role in the lethal effect of Pseudomonas against the intestinal epithelium (FIG. 25-2 in Appendix A). Mutant strains that lack PA-I, despite making abundant exotoxin A, have an attenuated effect on intestinal epithelial barrier function, and are completely apathogenic in a mouse model of lethal gut-derived sepsis. This is in dramatic contrast to the parental strain which is both highly disregulating against the intesitnal epithelium and 100% lethal.

The studies depicted in Appendix A were designed to determine whether the intestinal tract of a stressed host is a unique environment in which the virulence of P. aeruginosa is enhanced in vivo. In order to further investigate this question, a reporter strain of P. aeruginosa with GFP inserted downstream of the PA-I gene and the quorum sensing and RpoS promoters was constructed. As can be seen in FIG. 25-3 in Appendix A, these promoters increase PA-I expression in response to bacterial signaling pheromones, and the GFP reporter was highly responsive to these signals.

This reporter construct was used in studies that showed that PA-I is in vivo expressed in the intestinal lumen of mice subjected to 30% surgical hepatectomy by injecting bacteria directly into the cecum and retrieving strains 24 hours later (FIG. 25-4). In addition, PA-I is expressed within the lumen of mouse jejunum following segmental ischemia/reperfusion injury after injecting and retraining strains in an intestinal lumenal perfusion model. Because the blood from animals in both models failed to activate PA-I expression, it was hypothesized that the host-derived bacterial signaling factor was likely to be released locally by the intestinal epithelium itself.

The GFP PA-I reporter strains were exposed to filtered media from hypoxic cells, and were able to show a time dependent increase in PA-I expression (FIG. 25-5 in Appendix A). Results were confirmed by Northern blot (inset in FIG. 25-5).

To further understand how surgical stress and intestinal hypoxia might play a role in activating the virulence of P. aeruginosa, the role of HIF-1-a in this response was investigated. It is well known that hypoxia results in the accumulation of HIF-1-a in intestinal epithelial cells, as shown schematically in FIG. 25-6 in Appendix A.

Given the increasingly important role of HIF-1-a activation in intestinal epithelial homeostasis, experiments were designed to determine if HIF-1-a activation mediates the release of soluble compounds that activate P. aeruginosa virulence, as judged by expression of the PA-I lectin/adhesin.

An established Caco 2 cell line that had been stably transfected with HIF-1-a and its parental cell line were used. The method is outlined in FIGS. 25-7 to 25-8. Briefly, both cell lines were grown to confluence. Medium was collected and filtered through 0.22μ filters to remove any potential cellular components. Medium was then added to microtiter wells containing a fixed bacterial cell population of the GFP/PA-I reporter strain described above. Fluorescence was dynamically tracked over time and was calculated according to the formula given in FIG. 25-8 in Appendix A.

Results demonstrated a time-dependent induction of PA-I expression observed in GFP/PA-I reporter strains exposed to HIF-la medium, compared to control (FIG. 25-9 in appendix A). This finding were confirmed by Western blot analyses in reiterative experiments. HIF-1a activation was also confirmed by Western blot analysis (see inset in FIG. 25-9).

In order to identify the potential compounds that activate PA-I, the media from three groups of Caco-2 cells were examined, namely, control cells, Caco2 cells exposed to hypoxia, and Caco2 cells with forced expression of HIF-1a. The methods are briefly outlined in FIG. 25-10 in Appendix A. Each medium sample was separated into 4 molecular weight (MW) fractions, which were added to the microtiter plates containing the PA-I/GFP reporter strains and dynamic fluorimetry evaluated. Results from these experiments (shown in FIG. 25-11 of Appendix A) demonstrated that media fractions with MW of <3 kDa induced PA-I expression significantly.

Further studies were performed to show that HIF and hypoxic conditions have similar effects. Because of the MW of the potential inducing compound, genes known to be expressed in response to HIF-1-a activation were examined. Within this MW range potential candidate compounds related to nucleotide metabolism were identified. In particular, adenosine was of interest because it has been shown to be released in high concentrations following intestinal epithelial hypoxia and HIF-1-a activation.

As previously shown (see FIG. 25-12), adenosine accumulated in the media of intestinal epithelial cells exposed to hypoxia and/or HIF-1a activation, through a mechanism that involves upregulation of 5′-Nucleosidase (CD73) Activity (The mechanism is depicted in FIG. 25-13 of Appendix A and further elaborated in FIG. 25-20 of Appendix A). Based on these results, reiterative experiments were performed and media fractions were assayed for adenosine using HPLC/MS/MS, as outlined in FIG. 25-14 of Appendix A. Adenosine was greatly elevated in HIF-1-a-activated and hypoxic cell media (see FIG. 25-15 in Appendix A).

Further investigation revealed that the PA-I expression increase in the presence of adenosine was both dose- and time-dependent (FIG. 25-16 in Appendix A). Results were confirmed by Western blot (inset in FIG. 25-16 in Appendix A). For completeness, the effects of ATP, ADP, and AMP at similar concentrations were tested and revealed no evident inducing effects.

In order to determine if adenosine was the putative component within the media of HIF-1-a-activated Caco-2 cells that induced the expression of PA-I, adenosine deaminase was added to deplete the media of adenosine. Surprisingly, these experiments resulted in an even greater increase in PA-I expression, raising the possibility that a metabolite of adenosine, namely inosine, played a role in PA-I expression (FIG. 25-17 in Appendix A). Adenosine deaminase is expected to be present in P. aeruginosa based on its DNA sequence (FIG. 25-21 of Appendix A). Therefore, reiterative experiments were performed in the presence of varying concentrations of inosine. As can be seen in FIG. 25-18 of Appendix A, inosine induced PA-I expression at a concentration 10-fold less than the concentration of adenosine having an equivalent effect.

Reiterative experiments to directly compare the change in PA-I expression, over time, between inosine and adenosine demonstrated that not only was the effect of inosine greater, it also occurred at an earlier time point. Exemplary data showing that inosine induced PA-I expression at an earlier time point and at lower cell densities (OD) compared to adenosine are shown in FIG. 25-19 of Appendix A.

In conclusion, the present Example demonstrated that hypoxia or forced expression of HIF-1-a in Caco-2 cells resulted in the extracellular release of soluble compounds that activated the virulence circuitry of P. aeruginosa. Further, the data presented herein showed that adenosine and inosine may play important roles in this response. These results indicate that hypoxia and P. aeruginosa are involved in the activation or induction of a virulent phenotype against the intestinal epithelium via HIF-1-a activation. Without wishing to be bound by theory, one possible mechanism consistent with the data is depicted in FIG. 25-22 of Appendix A.

The following references have been cited throughout this disclosure and are hereby incorporated by reference in their entireties.
1. Laughlin et al. The key role of Pseudomonas aeruginosa PA-I lectin on experimental gut-derived sepsis Ann Surg. 232(1):133-42. 2000.
Alverdy et al Gut-derived sepsis occurs when the right pathogen with the right virulence genes meets the right host: evidence for in vivo virulence expression in Pseudomonas aeruginosa. Ann Surg. 232(4):480-489. 2000.
2. Rocha, Surgical stress shifts the intestinal Escherichia coli population to that of a more adherernt phenotype: role in barrier regulation. Surgery 2001 October;130(4):592
3. Alverdy et al. Influence of the critically ill state on host-pathogen interactions within the intestine: gut-derived sepsis redefined Crit. Care Med 31(2):598-607. 2003.
4. Wu et al. Pseudomonas aeruginosa expresses a lethal virulence determinant, the PA-I lectin/adheson, in the intestinal tract of a stressed host: the role of epithelia cell contact and molecules of the quorum sensing signalling system Ann.Surg. 238(5):754-64. 2003.
5. Wu et al. High-molecular-weight polyethylene glycol prevents lethal sepsis due to intestinal Pseudomonas aeruginosa Gastroenterology. 126(2):488-98. 2004.
6. Kohler et al. Components of intestinal epithelial hypoxia activate the virulence circuitry of Pseudomonas. Amer J Physiology:Gastro/Liver (accepted)
7. Zaborina et al. Bacterial nucleotide-interconverting enzymes and their role in host-pathogen interaction. Microbes and Infection (accepted)-2004
8. Zaborina et al. Antibiotic resistant clinical isolates of Pseudomonas aeruginosa isolates display extreme aggressiveness toward epithelial cells Infection and Immunity (submitted) 2004.
9. Wu et al. Surgical Infections (submitted) 2004.
10. Wu et al. Interferon-gamma binds to a 30 kDa membrane protein in Pseudomonas aeruginosa and induces the expression of a virulence related gene, the PA-I lectin. J Clinical Investigation (submitted)-2004
11. Zaborina et al. Opioid agonists signal Pseudomonas aeruginosa to express enhanced virulence through the action of the PA-I lectin. Infection and Immunity (submitted)-2004
1. Marshall J C. The ecology and immunology of the gastrointestinal tract in health and critical illness. J Hosp Infect 1991;19 Suppl C:7-17.
2. Marshall J C, Christou N V, Meakins J L. The gastrointestinal tract. The “undrained abscess” of multiple organ failure. Ann Surg 1993;218:111-9.
3. Angus D C, Wax R S. Epidemiology of sepsis: an update. Crit Care Med 2001 ;29:S109-16.
4. Barie P S. Surviving sepsis. Surg Infect (Larchmt) 2004;5:1-2.
5. Pastores S M, Katz D P, Kvetan V. Splanchnic ischemia and gut mucosal injury in sepsis and the multiple organ dysfunction syndrome. Am J Gastroenterol 1996;91 :1697-710.
6. Esposito S, Noviello S. [Bacterial infections in intensive care units: etiology and pathogenesis]. Infez Med 1997;5:145-59.
7. Murono K, Hirano Y, Koyano S, Ito K, Fujieda K. Molecular comparison of bacterial isolates from blood with strains colonizing pharynx and intestine in immunocompromised patients with sepsis. J Med Microbiol 2003;52:527-30.
8. Kropec A, Huebner J, Riffel M, Bayer U, Benzing A, Geiger K, Daschner F D. Exogenous or endogenous reservoirs of nosocomial Pseudomonas aeruginosa and Staphylococcus aureus infections in a surgical intensive care unit. Intensive Care Med 1993;19:161-5.
9. Neuhauser M M, Weinstein R A, Rydman R, Danziger L H, Karamn G, Quinn J P. Antibiotic resistance among gram-negative bacilli in US intensive care units: implications for fluoroquinolone use. Jama 2003;289:885-8.
10. Clark N M, Patterson J, Lynch J P, 3rd. Antimicrobial resistance among gram-negative organisms in the intensive care unit. Curr Opin Crit Care 2003;9:413-23.
11. Aliaga L, Mediavilla J D, Cobo F. A clinical index predicting mortality with Pseudomonas aeruginosa bacteraemia. J Med Microbiol 2002;51:615-9.
12. Arbo M D, Hariharan R, Nathan C, Barefoot L, Weinstein R A, Snydman D R. Utility of serial rectal swab cultures for detection of ceftazidime- and imipenem-resistant gram-negative bacilli from patients in the intensive care unit. Eur J Clin Microbiol Infect Dis 1998;17:727-30.
13. de Jonge E, Schultz M J, Spanjaard L, Bossuyt P M, Vroom M B, Dankert J, Kesecioglu J. Effects of selective decontamination of digestive tract on mortality and acquisition of resistant bacteria in intensive care: a randomised controlled trial. Lancet 2003;362:1011-6.
14. Laughlin R S, Musch M W, Hollbrook C J, Rocha F M, Chang E B, Alverdy J C. The key role of Pseudomonas aeruginosa PA-I lectin on experimental gut-derived sepsis. Ann Surg 2000;232:133-42.
15. Alverdy J, Holbrook C, Rocha F, Seiden L, Wu R L, Musch M, Chang E, Ohman D, Suh S. Gut-derived sepsis occurs when the right pathogen with the right virulence genes meets the right host: evidence for in vivo virulence expression in Pseudomonas aeruginosa. Ann Surg 2000;232:480-9.
16. Kurahashi K, Kajikawa O, Sawa T, Ohara M, Gropper M A, Frank D W, Martin T R, Wiener-Kronish J P. Pathogenesis of septic shock in Pseudomonas aeruginosa pneumonia. J Clin Invest 1999;104:743-50.
17. Fleiszig S M, Wiener-Kronish J P, Miyazaki H, Vallas V, Mostov K E, Kanada D, Sawa T, Yen T S, Frank D W. Pseudomonas aeruginosa-mediated cytotoxicity and invasion correlate with distinct genotypes at the loci encoding exoenzyme S. Infect Immun 1997;65:579-86.
18. Sawa T, Ohara M, Kurahashi K, Twining S S, Frank D W, Doroques D B, Long T, Gropper M A, Wiener-Kronish J P. In vitro cellular toxicity predicts Pseudomonas aeruginosa virulence in lung infections. Infect Immun 1998;66:3242-9.
19. Cioci G, Mitchell E P, Gautier C, Wimmerova M, Sudakevitz D, Perez S, Gilboa-Garber N, Imberty A. Structural basis of calcium and galactose recognition by the lectin PA-IL of Pseudomonas aeruginosa. FEBS Lett 2003;555:297-301.
20. Mitchell E, Houles C, Sudakevitz D, Wimmerova M, Gautier C, Perez S, Wu A M, Gilboa-Garber N, Imberty A. Structural basis for oligosaccharide-mediated adhesion of Pseudomonas aeruginosa in the lungs of cystic fibrosis patients. Nat Struct Biol 2002;9:918-21.
21. Kirkeby S, Moe D. Lectin interactions with alpha-galactosylated xenoantigens. Xenotransplantation 2002;9:260-7.
22. Glick J, Garber N. The intracellular localization of Pseudomonas aeruginosa lectins. J Gen Microbiol 1983;129 (Pt 10):3085-90.
23. Bajolet-Laudinat O, Girod-de Bentzmann S, Tournier J M, Madoulet C, Plotkowski M C, Chippaux C, Puchelle E. Cytotoxicity of Pseudomonas aeruginosa internal lectin PA-I to respiratory epithelial cells in primary culture. Infect Immun 1994;62:4481-7.
24. Wu L, Holbrook C, Zaborina O, Ploplys E, Rocha F, Pelham D, Chang E, Musch M, Alverdy J. Pseudomonas aeruginosa expresses a lethal virulence determinant, the PA-I lectin/adhesin, in the intestinal tract of a stressed host: the role of epithelia cell contact and molecules of the Quorum Sensing Signaling System. Ann Surg 2003;238:754-64.
25. Pennisi E. Infectious disease. Cholera strengthened by trip through gut. Science 2002;296: 1783-4.
26. Schuster M, Hawkins A C, Harwood C S, Greenberg E P. The Pseudomonas aeruginosa RpoS regulon and its relationship to quorum sensing. Mol Microbiol 2004;51:973-85.
27. Dunlap P V. Quorum regulation of luminescence in Vibrio-fischeri. J Mol Microbiol Biotechnol 1999;1:5-12.
28. Smith R S, Iglewski B H. P. aeruginosa quorum-sensing systems and virulence. Curr Opin Microbiol 2003;6:56-60.
29. Whistler C A, Corbell N A, Sarniguet A, Ream W, Loper J E. The two-component regulators GacS and GacA influence accumulation of the stationary-phase sigma factor sigmaS and the stress response in Pseudomonas fluorescens Pf-5. J Bacteriol 1998;180:6635-41.
30. Winzer K, Falconer C, Garber N C, Diggle S P, Camara M, Williams P. The Pseudomonas aeruginosa lectins PA-IL and PA-IIL are controlled by quorum sensing and by RpoS. J Bacteriol 2000;182:6401-11.
31. Winzer K, Williams P. Quorum sensing and the regulation of virulence gene expression in pathogenic bacteria. Int J Med Microbiol 2001 ;291:131-43.
32. Winzer K, Hardie K R, Burgess N, Doherty N, Kirke D, Holden M T, Linforth R, Cornell K A, Taylor A J, Hill P J, Williams P. LuxS: its role in central metabolism and the in vitro synthesis of 4-hydroxy-5-methyl-3(2H)-furanone. Microbiology 2002; 148:909-22.
33. Diggle S P, Winzer K, Lazdunski A, Williams P, Camara M. Advancing the quorum in Pseudomonas aeruginosa: MvaT and the regulation of N-acylhomoserine lactone production and virulence gene expression. J Bacteriol 2002;184:2576-86.
34. Diggle S P, Winzer K, Chhabra S R, Worrall K E, Camara M, Williams P. The Pseudomonas aeruginosa quinolone signal molecule overcomes the cell density-dependency of the quorum sensing hierarchy, regulates rhi-dependent genes at the onset of stationary phase and can be produced in the absence of LasR. Mol Microbiol 2003;50:29-43.
35. Goodman A L, Lory S. Analysis of regulatory networks in Pseudomonas aeruginosa by genomewide transcriptional profiling. Curr Opin Microbiol 2004;7:39-44.
36. Wagner V E, Bushnell D, Passador L, Brooks A I, Iglewski B H. Microarray analysis of Pseudomonas aeruginosa quorum-sensing regulons: effects of growth phase and environment. J Bacteriol 2003;185:2080-95.
37. Fenchel T. Microbial behavior in a heterogeneous world. Science 2002;296:1068-71.
38. Juhas M, Wiehlmann L, Huber B, Jordan D, Lauber J, Salunkhe P, Limpert A S, von Gotz F, Steinmetz I, Eberl L, Tummler B. Global regulation of quorum sensing and virulence by VqsR in Pseudomonas aeruginosa. Microbiology 2004; 150:831-41.
39. Koch B, Nielsen T H, Sorensen D, Andersen J B, Christophersen C, Molin S, Givskov M, Sorensen J, Nybroe O. Lipopeptide production in Pseudomonas sp. strain DSS73 is regulated by components of sugar beet seed exudate via the Gac two-component regulatory system. Appl Environ Microbiol 2002;68:4509-16.
40. Sansonetti P. Host-pathogen interactions: the seduction of molecular cross talk. Gut 2002;50 Suppl 3:1112-8.
41. Lory S, Wolfgang M, Lee V, Smith R. The multi-talented bacterial adenylate cyclases. Int J Med Microbiol 2004;293:479-82.
42. Parkins M D, Ceri H, Storey D G. Pseudomonas aeruginosa GacA, a factor in multihost virulence, is also essential for biofilm formation. Mol Microbiol 2001;40:1215-26.
43. Wolfgang M C, Lee V T, Gilmore M E, Lory S. Coordinate regulation of bacterial virulence genes by a novel adenylate cyclase-dependent signaling pathway. Dev Cell 2003;4:253-63.
44. Venturi V. Control of rpoS transcription in Escherichia coli and Pseudomonas: why so different? Mol Microbiol 2003;49:1-9.
45. Fagerlind M G, Rice S A, Nilsson P, Harlen M, James S, Charlton T, Kjelleberg S. The role of regulators in the expression of quorum-sensing signals in Pseudomonas aeruginosa. J Mol Microbiol Biotechnol 2004;6:88-100.
46. Bertani I, Sevo M, Kojic M, Venturi V. Role of GacA, LasI, RhlI, Ppk, PsrA, Vfr and ClpXP in the regulation of the stationary-phase sigma factor rpoS/RpoS in Pseudomonas. Arch Microbiol 2003;180:264-71.
47. Smith R S, Wolfgang M C, Lory S. An adenylate cyclase-controlled signaling network regulates Pseudomonas aeruginosa virulence in a mouse model of acute pneumonia. Infect Inmun 2004;72:1677-84.
48. Chen C, Brown D R, Xie Y, Green B T, Lyte M. Catecholamines modulate Escherichia coli 0157:H7 adherence to murine cecal mucosa. Shock 2003;20:183-8.
49. Lyte M, Freestone P P, Neal C P, Olson B A, Haigh R D, Bayston R, Williams P H. Stimulation of Staphylococcus epidermidis growth and biofilm formation by catecholamine inotropes. Lancet 2003;361:130-5.
50. Yoshida S, Ohta J, Yamasaki K, Kamei H, Harada Y, Yahara T, Kaibara A, Ozaki K, Tajiri T, Shirouzu K. Effect of surgical stress on endogenous morphine and cytokine levels in the plasma after laparoscopoic or open cholecystectomy. Surg Endosc 2000;14:137-40.
51. Brix-Christensen V, Tonnesen E, Sanchez R G, Bilfinger T V, Stefano G B. Endogenous morphine levels increase following cardiac surgery as part of the antiinflammatory response? Int J Cardiol 1997;62:191-7.
52. Bruce N C, Wilmot C J, Jordan K N, Trebilcock A E, Gray Stephens L D, Lowe C R. Microbial degradation of the morphine alkaloids: identification of morphine as an intermediate in the metabolism of morphine by Pseudomonas putida M10. Arch Microbiol 1990;154:465-70.
53. Hailes A M, Bruce N C. Biological synthesis of the analgesic hydromorphone, an intermediate in the metabolism of morphine, by Pseudomonas putida M10. Appl Environ Microbiol 1993;59:2166-70.
54. Aballay A, Ausubel F M. Caenorhabditis elegans as a host for the study of host-pathogen interactions. Curr Opin Microbiol 2002;5:97-101.
55. Tan M W, Mahajan-Miklos S, Ausubel F M. Killing of Caenorhabditis elegans by Pseudomonas aeruginosa used to model mammalian bacterial pathogenesis. Proc Natl Acad Sci U S A 1999;96:715-20.
56. Hendrickson E L, Plotnikova J, Mahajan-Miklos S, Rahme L G, Ausubel F M. Differential roles of the Pseudomonas aeruginosa PA14 rpoN gene in pathogenicity in plants, nematodes, insects, and mice. J Bacteriol 2001;183:7126-34.
57. Lai H P, Hsueh P R, Chen Y C, Lee P I, Lu C Y, Lu M Y, Lin W C, Hsieh Y C, Lee C Y, Lin K H, Huang L M. Bacteremia in hematological and oncological children with febrile neutropenia: experience in a tertiary medical center in Taiwan. J Microbiol Immunol Infect 2003;36:197-202.
58. Chiu H H, Huang L M, Lee P I, Lee C Y. Bacteremia and fungemia in hematological and oncological children with neutropenic fever: two-year study in a medical center. J Microbiol Immunol Infect 1998;31:101-6.
59. Livermore D M. The threat from the pink corner. Ann Med 2003;35:226-34.
60. Osmon S, Ward S, Fraser V J, Kollef M H. Hospital mortality for patients with bacteremia due to Staphylococcus aureus or Pseudomonas aeruginosa. Chest 2004;125:607-16.
61. Srinivasan A, Wolfenden L L, Song X, Mackie K, Hartsell T L, Jones H D, Diette G B, Orens J B, Yung R C, Ross T L, Merz W, Scheel P J, Haponik E F, Perl T M. An outbreak of Pseudomonas aeruginosa infections associated with flexible bronchoscopes. N Engl J Med 2003;348:221-7.
62. Gouvea E F, Branco R C, Monteiro R C, Halpern M, Ribeiro-Filho J, Silveira V G, Tavares G C, Rodrigues M S, Coelho H S, Basto S T, Santoro-Lopes G. Outcome of infections caused by multiple drug-resistant bacteria in liver transplant recipients. Transplant Proc 2004;36:958-60.
63. Nusrat A, Turner J R, Madara J L. Molecular physiology and pathophysiology of tight junctions. IV. Regulation of tight junctions by extracellular stimuli: nutrients, cytokines, and immune cells. Am J Physiol Gastrointest Liver Physiol 2000;279:G851-7.
64. Stefano G B, Zhu W, Cadet P, Bilfinger T V, Mantione K. Morphine enhances nitric oxide release in the mammalian gastrointestinal tract via the micro(3) opiate receptor subtype: a hormonal role for endogenous morphine. J Physiol Pharmacol 2004;55:279-88.
65. Eisenstein L K, MacFarland A S, Peng X, Hilburger M E, Rahim R T, Meissler L J, Jr., Rogers T J, Wan A C, Adler M W. Effect of opioids on oral Salmonella infection and immune function. Adv Exp Med Biol 2001;493:169-76.
66. Parsek M R, Greenberg E P. Quorum sensing signals in development of Pseudomonas aeruginosa biofilms. Methods Enzymol 1999;3 10:43-55.
67. Yarwood J M, Bartels D J, Volper E M, Greenberg E P. Quorum sensing in Staphylococcus aureus biofilms. J Bacteriol 2004; 186:1838-50.
68. Jacobs M A, Alwood A, Thaipisuttikul I, Spencer D, Haugen E, Ernst S, Will O, Kaul R, Raymond C, Levy R, Chun-Rong L, Guenthner D, Bovee D, Olson M V, Manoil C. Comprehensive transposon mutant library of Pseudomonas aeruginosa. Proc Natl Acad Sci U S A 2003;100:14339-44.
69. Schuster M, Lostroh C P, Ogi T, Greenberg E P. Identification, timing, and signal specificity of Pseudomonas aeruginosa quorum-controlled genes: a transcriptome analysis. J Bacteriol 2003;185:2066-79.
70. Ledgham F, Ventre I, Soscia C, Foglino M, Sturgis J N, Lazdunski A. Interactions of the quorum sensing regulator QscR: interaction with itself and the other regulators of Pseudomonas aeruginosa LasR and RhlR. Mol Microbiol 2003;48:199-210.
71. Whiteley M, Lee K M, Greenberg E P. Identification of genes controlled by quorum sensing in Pseudomonas aeruginosa. Proc Natl Acad Sci U S A 1999;96:13904-9.
72. Whiteley M, Parsek M R, Greenberg E P. Regulation of quorum sensing by RpoS in Pseudomonas aeruginosa. J Bacteriol 2000;1 82:4356-60.
73. Whiteley M, Greenberg E P. Promoter specificity elements in Pseudomonas aeruginosa quorum-sensing-controlled genes. J Bacteriol 2001; 183:5529-34.
74. Bolin I, Lonroth H, Svennerholm A M. Identification of Helicobacter pylori by immunological dot blot method based on reaction of a species-specific monoclonal antibody with a surface-exposed protein. J Clin Microbiol 1995;33:381-4.
75. Rojas M, Conway P L. A dot-blot assay for adhesive components relative to probiotics. Methods Enzymol 2001;336:389-402.
76. Matta H, Punj V, Kanwar S S. An immuno-dot blot assay for detection of thermostable protease from Pseudomonas sp. AFT-36 of dairy origin. Lett Appl Microbiol 1997;25 :300-2.
77. Min D H, Tang W J, Mrksich M. Chemical screening by mass spectrometry to identify inhibitors of anthrax lethal factor. Nat Biotechnol 2004;22:717-23.
78. Autar R, Khan A S, Schad M, Hacker J, Liskamp R M, Pieters R J. Adhesion inhibition of F1C-fimbriated Escherichia coli and Pseudomonas aeruginosa PAK and PAO by multivalent carbohydrate ligands. Chembiochem 2003;4: 1317-25.
79. Davies A G, Pierce-Shimomura J T, Kim H, VanHoven M K, Thiele T R, Bonci A, Bargmann C I, McIntire S L. A central role of the BK potassium channel in behavioral responses to ethanol in C. elegans. Cell 2003;1 15:655-66.
80. Davies A G, Bettinger J C, Thiele T R, Judy M E, McIntire S L. Natural Variation in the npr-1 Gene Modifies Ethanol Responses of Wild Strains of C. elegans. Neuron 2004;42:731-43.
81. Davies A G, McIntire S L. Using C. elegans to screen for targets of ethanol and behavior-altering drugs. Biol Proced Online 2004;6:113-119.
82. Anderson G L, Cole R D, Williams P L. Assessing behavioral toxicity with Caenorhabditis elegans. Environ Toxicol Chem 2004;23:1235-40.
83. Westerman B A, Poutsma A, Steegers E A, Oudejans C B. C2360, a nuclear protein expressed in human proliferative cytotrophoblasts, is a representative member of a novel protein family with a conserved coiled coil-helix-coiled coil-helix domain. Genomics 2004;83:1094-104.
84. Uccelletti D, O'Callaghan C, Berninsone P, Zemtseva I, Abeijon C, Hirschberg C B. ire-1-dependent transcriptional up-regulation of a lumenal uridine diphosphatase from Caenorhabditis elegans. J Biol Chem 2004;279:27390-8.
85. Matsko N, Mueller M. AFM of biological material embedded in epoxy resin. J Struct Biol 2004;146:334-43.
86. Smith M G, Des Etages S G, Snyder M. Microbial synergy via an ethanol-triggered pathway. Mol Cell Biol 2004;24:3874-84.
87. Boonstra B, Rathbone D A, Bruce N C. Engineering novel biocatalytic routes for production of semisynthetic opiate drugs. Biomol Eng 2001;18:41-7.
88. Neves F O, Ho P L, Raw I, Pereira C A, Moreira C, Nascimento A L. Overexpression of a synthetic gene encoding human alpha interferon in Escherichia coli. Protein Expr Purif 2004;35:353-9.
89. French C E, Bruce N C. Purification and characterization of morphinone reductase from Pseudomonas putida M10. Biochem J 1994;301 (Pt 1):97-103.
90. Steidler L, Hans W, Schotte L, Neirynck S, Obermeier F, Falk W, Fiers W, Remaut E. Treatment of murine colitis by Lactococcus lactis secreting interleukin-10. Science 2000;289: 1352-5.
91. Wittert G, Hope P, Pyle D. Tissue distribution of opioid receptor gene expression in the rat. Biochem Biophys Res Commun 1996;218:877-81.
92. Goumon Y, Casares F, Pryor S, Ferguson L, Brownawell B, Cadet P, Rialas C M, Welters I D, Sonetti D, Stefano G B. Ascaris suum, an intestinal parasite, produces morphine. J Immunol 2000;165:339-43.
93. Trebichavsky I, Splichal I, Splichalova A, Muneta Y, Mori Y. Systemic and local cytokine response of young piglets to oral infection with Salmonella enterica serotype Typhimurium. Folia Microbiol (Praha) 2003;48:403-7.
94. Braun F, Hosseini M, Wieland E, Sattler B, Muller A R, Fandrich F, Kremer B, Ringe B. Kinetics and localization of interleukin-2, interleukin-6, heat shock protein 70, and interferon gamma during intestinal-rerfusion injury. Transplant Proc 2004;36:267-9.
95. Golden T R, Hinerfeld D A, Melov S. Oxidative stress and aging: beyond correlation. Aging Cell 2002; 1:117-23.
96. Schultz M J, Rijneveld A W, Speelman P, van Deventer S J, van der Poll T. Endogenous interferon-gamma impairs bacterial clearance from lungs during Pseudomonas aeruginosa pneumonia. Eur Cytokine Netw 2001;12:39-44.
97. Murphey E D, Lin C Y, McGuire R W, Toliver-Kinsky T, Herndon D N, Sherwood E R. Diminished bacterial clearance is associated with decreased IL-12 and interferon-gamma production but a sustained proinflammatory response in a murine model of postseptic immunosuppression. Shock 2004;21:415-25.
98. Ojielo C I, Cooke K, Mancuso P, Standiford T J, Olkiewicz K M, Clouthier S, Corrion L, Ballinger M N, Toews G B, Paine R, 3rd, Moore B B. Defective phagocytosis and clearance of Pseudomonas aeruginosa in the lung following bone marrow transplantation. J Immunol 2003;171:4416-24.
99. Moser C, Jensen P O, Kobayashi O, Hougen H P, Song Z, Rygaard J, Kharazmi A, N Hb. Improved outcome of chronic Pseudomonas aeruginosa lung infection is associated with induction of a Th1-dominated cytokine response. Clin Exp Immunol 2002;127:206-13.
100. Yu H, Nasr S Z, Deretic V. Innate lung defenses and compromised Pseudomonas aeruginosa clearance in the malnourished mouse model of respiratory infections in cystic fibrosis. Infect Immun 2000;68:2142-7.
101. Clauser K R, Baker P, Burlingame A L. Role of accurate mass measurement (±10 ppm) in protein identification strategies employing MS or MS/MS and database searching. Anal Chem 1999;71:2871-82.
102. Dancik V, Addona T A, Clauser K R, Vath J E, Pevzner P A. De novo peptide sequencing via tandem mass spectrometry. J Comput Biol 1999;6:327-42.

Numerous modifications and variations of the invention are possible in view of the above teachings and are within the scope of the invention. The entire disclosures of all publications cited herein are hereby incorporated by reference.

1. A method of screening for a modulator of an epithelial cell barrier function comprising
(a) contacting a PA-I lectin/adhesin and an epithelial cell in the presence and absence of a candidate modulator;
(b) measuring epithelial cell barrier function; and
(c) identifying the candidate modulator as a modulator of an epithelial cell barrier function if the barrier function in the presence of said candidate modulator differs from the barrier function in the absence of said candidate modulator.

2. A method of screening for a modulator of an epithelial cell barrier function comprising
(a) contacting a candidate modulator and an epithelial cell releasing a bacterial signaling compound selected from the group consisting of an opioid, an opioid receptor agonist and interferon-γ;
(b) measuring the release of the bacterial signaling compound in the presence or absence of the candidate modulator; and
(c) identifying the candidate modulator as a modulator of an epithelial cell barrier function if the level of signal transmission in the presence of the candidate modulator differs from the level of signal transmission in the absence of the candidate modulator.

3. The method according to claim 2 wherein the microbial pathogen is Pseudomonas aeruginosa.

4. The method according to claim 2 wherein the modulator inhibits signal transmission by interacting with the bacterial signaling compound external to the microbial pathogen.

5. The method according to claim 4 wherein the modulator binds to the bacterial signaling compound.

6. The method according to claim 4 wherein the modulator binds to the cell surface of a microbial pathogen.

7. A method of screening for a modulator of an epithelial cell barrier function comprising
(a) contacting a candidate modulator and a microbial pathogen comprising a functional mvfR coding region under conditions wherein the mvfR is expressed;
(b) measuring MvfR expression in the presence or absence of the candidate modulator; and
(c) identifying the candidate modulator as a modulator of an epithelial cell barrier function if the level of MvfR in the presence of the candidate modulator differs from the level of MvfR in the absence of the candidate modulator.

8. The method according to claim 7 wherein the microbial pathogen is Pseudomonas aeruginosa.

9. The method according to claim 7 wherein MvfR expression is measured by determining the level of MvfR activity.

10. The method according to claim 7 wherein MvfR expression is measured by determining the level of expression of a coding region regulated by MvfR.

11. The method according to claim 10 wherein the coding region regulated by MvfR is selected from the group consisting of a chimeric coding region and a heterologous coding region.

12. The method according to claim 7 wherein the modulator inhibits expression of MvfR.

13. The method according to claim 7 wherein the conditions comprise exposure to a compound selected from the group consisting of an opioid, an opioid receptor agonist and an interferon-γ.

14. A method of modulating the activity of MvfR comprising administering a therapeutically effective amount of the modulator according to claim 7.

15. The method of claim 14 wherein said administering comprises direct introduction of the modulator into the intestine of an organism, wherein the intestine is populated by a Pseudomonas aeruginosa cell.

16. The method of claim 15 further comprising administering a high molecular weight polyethylene glycol-like compound.

17. A method of screening for a modulator of an epithelial cell barrier function comprising
(a) contacting a candidate modulator and a microbial pathogen comprising a functional PA-I lectin/adhesin coding region under conditions wherein the PA-I lectin/adhesin is expressed;
(b) measuring PA-I lectin/adhesin expression in the presence or absence of the candidate modulator; and
(c) identifying the candidate modulator as a modulator of an epithelial cell barrier function if the level of PA-I lectin/adhesin in the presence of the candidate modulator differs from the level of PA-I lectin/adhesin in the absence of the candidate modulator.

18. The method according to claim 17 wherein the microbial pathogen is Pseudomonas aeruginosa.

19. The method according to claim 17 wherein the modulator inhibits PA-I lectin/adhesin expression.

20. The method according to claim 17 wherein the conditions comprise induction of PA-I lectin/adhesin expression by a compound selected from the group consisting of an opioid, an opioid receptor agonist and interferon-γ.

21. The method according to claim 17 wherein the modulator interferes with interaction between OprF and interferon-γ.

22. The method according to claim 17 wherein the modulator interferes with the interaction between an opioid receptor and a compound selected from the group consisting of an opioid and an opioid receptor agonist.

23. The method according to claim 22 wherein the opioid receptor agonist is a κ-opioid agonist selected from the group consisting of U-50488, U-69593, enadoline, ethylketocyclazocine, salvinorin A and asimadoline.

24. A method of modulating the expression of PA-I lectin/adhesin comprising administering a therapeutically effective amount of the modulator according to claim 14.

25. The method of claim 24 wherein said administering comprises direct introduction of the modulator into the intestine of an organism, wherein the intestine is populated by a Pseudomonas aeruginosa cell.

26. The method of claim 25 further comprising administering a high molecular weight polyethylene glycol-like compound.

27. The method according to any one of claims 1, 2, 7, and 17 wherein said barrier function is assessed by microscopic examination of a cellular junction formed between at least two epithelial cells.

28. The method according to any one of claims 1, 2, 7, and 17 wherein the epithelial cell barrier function is measured by transepithelial cell electrical resistance (TEER).

29. The method according to claim 28 wherein said TEER is higher in the presence of said candidate modulator than in the absence of said candidate modulator.

30. A method of treating a disorder characterized by an epithelial cell barrier dysfunction comprising administering to an organism in need thereof a therapeutically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-1 antagonist, an endomorphine-2 antagonist, an antagonist to δ opioid agonist BW373U86 and the modulator according to claim 1.

31. The method according to claim 30 wherein the opioid receptor antagonist is an antagonist of a κ-opioid receptor agonist selected from the group consisting of U-50488, U-69593, enadoline, ethylketocyclazocine, salvinorin A and asimadoline.

32. The method according to claim 30 wherein the opioid receptor antagonist is selected from the group consisting of nor-binaltorphimine, 5′-guanidinonaltrindole, nalmefine, naltrindole, an indolmorphinan, naltrexone and MR2266 ([(−)-(1R,5R,9R)-5,9-diethyl-2-(3-furylmethyl)-2′-hydroxy-6,7-benzomorphan).

33. The method according to claim 30 wherein said organism is a human patient.

34. The method according to claim 30 wherein the disorder is selected from the group consisting of gut-derived sepsis, a burn injury, neonatal necrotizing enterocolitis, infection associated with severe neutropenia, toxic colitis, inflammatory bowel disease, irritable bowel syndrome, enteropathy, transplant rejection, pouchitis, pig belly, Pseudomonas-mediated ophthalmologic infection, Pseudomonas-mediated otologic infection and Pseudomonas-mediated cutaneous infection.

35. The method according to claim 30 further comprising administration of a biocompatible polymer.

36. The method according to claim 30 further comprising a high molecular weight polyethylene glycol-like compound.

37. The method according to claim 36 wherein the high molecular weight polyethylene glycol-like compound is polyethylene glycol having an average molecular weight of at least 15 kilodaltons.

38. A method of reducing the risk of developing a disorder characterized by an epithelial cell barrier dysfunction comprising administering to an organism at risk of developing said disorder a prophylactically effective amount of a compound selected from the group consisting of an opioid receptor antagonist, an interferon-γ antagonist, an MvfR antagonist, a regulator of MvfR expression, a PA-I antagonist, a negative regulator of PA-I expression, an endomorphine-1 antagonist, an endomorphine-2 antagonist, an antagonist to δ opioid agonist BW373U86 and the modulator according to claim 1.

39. The method according to claim 38 wherein the opioid receptor antagonist selected from the group consisting of a U-50,488 antagonist, a U-69,593 antagonist, an enadoline antagonist, an ethylketocyclazocine antagonist, a salvinorin A antagonist, an asimadoline antagonist, nor-binaltorphimine, 5′-guanidinonaltrindole, nalmefine, naltrindole, an indolmorphinan, naltrexone and MR2266 ([(−)-(1R,5R,9R)-5,9-diethyl-2-(3-furylmethyl)-2′-hydroxy-6,7-benzomorphan.

40. The method according to claim 38 wherein said organism is a human patient.

41. The method according to claim 38 wherein the disorder is selected from the group consisting of gut-derived sepsis, a burn injury, neonatal necrotizing enterocolitis, infection associated with severe neutropenia, toxic colitis, inflammatory bowel disease, irritable bowel syndrome, enteropathy, transplant rejection, pouchitis, pig belly, Pseudomonas-mediated ophthalmologic infection, Pseudomonas-mediated otologic infection and Pseudomonas-mediated cutaneous infection.

42. The method according to claim 38 further comprising administration of a high molecular weight polyethylene glycol-like compound.

43. The method according to claim 42 wherein the high molecular weight polyethylene glycol-like compound is polyethylene glycol having an average molecular weight of at least 15 kilodaltons.

44. A method of reducing a symptom associated with an epithelial cell barrier disorder, comprising administering to a patient in need thereof a compound selected from the group consisting of an opioid receptor antagonist, an interferon-γ antagonist, an MvfR antagonist, a regulator of MvfR expression, a PA-I antagonist, a regulator of PA-I expression, an endomorphine-1 antagonist, an endomorphine-2 antagonist, an antagonist to δ opioid agonist BW373U86 and the modulator according to claim 1, wherein the compound is administered in an amount effective to reduce at least one symptom of said disorder.

45. The method according to claim 44 wherein the opioid receptor antagonist is an antagonist of a K-opioid receptor agonist selected from the group consisting of U-50488, U-69593, enadoline, ethylketocyclazocine, salvinorin A and asimadoline.

46. The method according to claim 44 wherein the opioid receptor antagonist is selected from the group consisting of nor-binaltorphimine, 5′-guanidinonaltrindole, nalmefine, naltrindole, an indolmorphinan, naltrexone and MR2266 ([(−)-(1R,5R,9R)-5,9-diethyl-2-(3-furylmethyl)-2′-hydroxy-6,7-benzomorphan).

47. The method according to claim 44 further comprising administration of a high molecular weight polyethylene glycol-like compound.

48. The method according to claim 47 wherein the high molecular weight polyethylene glycol-like compound is polyethylene glycol having an average molecular weight of at least 15 kilodaltons.

49. An isolated modulator identified by the method according to claim 1.

50. A composition comprising the modulator according to claim 49 and a high molecular weight polyethylene glycol-like compound.

51. An article of manufacture comprising a label packaging material and an effective amount of the modulator according to claim 49, wherein the packaging material comprises a label or package insert indicating that said modulator can be used for treating, ameliorating, or preventing an epithelial cell barrier disorder.

52. The article of manufacture according to claim 51 wherein said disorder is selected from the group consisting of gut-derived sepsis, a burn injury, neonatal necrotizing enterocolitis, infection associated with severe neutropenia, toxic colitis, inflammatory bowel disease, irritable bowel syndrome, enteropathy, transplant rejection, pouchitis, pig belly, Pseudomonas-mediated ophthalmologic infection, Pseudomonas-mediated otologic infection and Pseudomonas-mediated cutaneous infection.

53. A method of using the modulator according to claim 49 in the preparation of a medicament for treating, ameliorating, or preventing a disorder selected from the group consisting of gut-derived sepsis, a burn injury, neonatal necrotizing enterocolitis, infection associated with severe neutropenia, toxic colitis, inflammatory bowel disease, irritable bowel syndrome, enteropathy, transplant rejection, pouchitis, pig belly, Pseudomonas-mediated ophthalmologic infection, Pseudomonas-mediated otologic infection and Pseudomonas-mediated cutaneous infection.


SEQUENCE LISTING (2 sequences)

SEQ ID NO: 1 - DNA, 27 bp, Artificial sequence
  Synthetic primer
tctagaactagtggatccccgcggatg

SEQ ID NO: 2 - DNA, 27 bp, Artificial sequence
  Synthetic primer
gcagactaggtcgacaagcttgatatc